SEQUENCE LISTING (5 sequences)

SEQ ID NO: 1 - DNA, 1311 bp, Homo sapiens
atggaccgcggcacgctccctctggctgttgccctgctgctggccagctgcagcctcagccccacaagtcttgcagaaacagtccattgtgaccttcagcctgtgggccccgagagggacgaggtgacatataccactagccaggtctcgaagggctgcgtggctcaggcccccaatgccatccttgaagtccatgtcctcttcctggagttcccaacgggcccgtcacagctggagctgactctccaggcatccaagcaaaatggcacctggccccgagaggtgcttctggtcctcagtgtaaacagcagtgtcttcctgcatctccaggccctgggaatcccactgcacttggcctacaattccagcctggtcaccttccaagagcccccgggggtcaacaccacagagctgccatccttccccaagacccagatccttgagtgggcagctgagaggggccccatcacctctgctgctgagctgaatgacccccagagcatcctcctccgactgggccaagcccaggggtcactgtccttctgcatgctggaagccagccaggacatgggccgcacgctcgagtggcggccgcgtactccagccttggtccggggctgccacttggaaggcgtggccggccacaaggaggcgcacatcctgagggtcctgccgggccactcggccgggccccggacggtgacggtgaaggtggaactgagctgcgcacccggggatctcgatgccgtcctcatcctgcagggtcccccctacgtgtcctggctcatcgacgccaaccacaacatgcagatctggaccactggagaatactccttcaagatctttccagagaaaaacattcgtggcttcaagctcccagacacacctcaaggcctcctgggggaggcccggatgctcaatgccagcattgtggcatccttcgtggagctaccgctggccagcattgtctcacttcatgcctccagctgcggtggtaggctgcagacctcacccgcaccgatccagaccactcctcccaaggacacttgtagcccggagctgctcatgtccttgatccagacaaagtgtgccgacgacgccatgaccctggtactaaagaaagagcttgttgcgcatttgaagtgcaccatcacgggcctgaccttctgggaccccagctgtgaggcagaggacaggggtgacaagtttgtcttgcgcagtgcttactccagctgtggcatgcaggtgtcagcaagtatgatcagcaatgaggcggtggtcaatatcctgtcgagctcatcaccacagcgg

SEQ ID NO: 2 - protein, 437 aa, Homo sapiens
MDRGTLPLAVALLLASCSLSPTSLAETVHCDLQPVGPERGEVTYTTSQVSKGCVAQAPNAILEVHVLFLEFPTGPSQLELTLQASKQNGTWPREVLLVLSVNSSVFLHLQALGIPLHLAYNSSLVTFQEPPGVNTTELPSFPKTQILEWAAERGPITSAAELNDPQSILLRLGQAQGSLSFCMLEASQDMGRTLEWRPRTPALVRGCHLEGVAGHKEAHILRVLPGHSAGPRTVTVKVELSCAPGDLDAVLILQGPPYVSWLIDANHNMQIWTTGEYSFKIFPEKNIRGFKLPDTPQGLLGEARMLNASIVASFVELPLASIVSLHASSCGGRLQTSPAPIQTTPPKDTCSPELLMSLIQTKCADDAMTLVLKKELVAHLKCTITGLTFWDPSCEAEDRGDKFVLRSAYSSCGMQVSASMISNEAVVNILSSSSPQR

SEQ ID NO: 3 - DNA, 30 bp, Artificial Sequence
  Primer
acgaagcttgaaacagtccattgtgacctt

SEQ ID NO: 4 - DNA, 29 bp, Artificial Sequence
  Primer
ttagatatctggcctttgcttgtgcaacc

SEQ ID NO: 5 - protein, 633 aa, Homo sapiens
ETVHCDLQPVGPERGEVTYTTSQVSKGCVAQAPNAILEVHVLFLEFPTGPSQLELTLQASKQNGTWPREVLLVLSVNSSVFLHLQALGIPLHLAYNSSLVTFQEPPGVNTTELPSFPKTQILEWAAERGPITSAAELNDPQSILLRLGQAQGSLSFCMLEASQDMGRTLEWRPRTPALVRGCHLEGVAGHKEAHILRVLPGHSAGPRTVTVKVELSCAPGDLDAVLILQGPPYVSWLIDANHNMQIWTTGEYSFKIFPEKNIRGFKLPDTPQGLLGEARMLNASIVASFVELPLASIVSLHASSCGGRLQTSPAPIQTTPPKDTCSPELLMSLIQTKCADDAMTLVLKKELVAHLKCTITGLTFWDPSCEAEDRGDKFVLRSAYSSCGMQVSASMISNEAVVNILSSSSPQRKKVHCLNMDSLSFQLGLYLSPHFLQASNTIEPGQQSFVQVRVSPSVSEFLLQLDSCHLDLGPEGGTVELIQGRAAKGNCVSLLSPSPEGDPRFSFLLHFYTVPIPKTGTLSCTVALRPKTGSQDQEVHRTVFMRLNIISPDLSGCTSKGLVLPAVLGITFGAFLIGALLTAALWYIYSHTRSPSKREPVVAVAAPASSESSSTNHSIGSTQSTPCSTSSMA

(19) United States
(12) Patent Application Publication
(10) Pub. No.: US 2009/0286271 A1
(43) Pub. Date: Nov. 19, 2009

(54) Methods of Diagnosing and Treating Complications of Pregnancy

(76) Inventors: Ananth S. Karumanchi, Chestnut Hill, MA (US); Vikas P. Sukhatme, Newton, MA (US); Mourad Toporsian, Toronto (CA); Michelle V. Letarte, Toronto (CA)

Correspondence Address: CLARK & ELBING LLP, 101 FEDERAL STREET, BOSTON, MA, 02110, US

(21) Appl. No.: 12/227,658

(22) PCT Filed: May 31, 2007
(86) PCT No.: PCT/US07/12787

§ 371 Date: Nov. 24, 2008

Related U.S. Application Data
(63) Continuation-in-part of application No. 11/443,920, filed on May 31, 2006.
(60) Provisional application No. 60/852,761, filed on Oct. 19, 2006.

(51) Int. Cl. C12Q 1/02; G01N 33/00

(52) U.S. Cl. 435/29; 436/86

(57) ABSTRACT

Disclosed herein are methods for treating a pregnancy related hypertensive disorder, such as pre-eclampsia and eclampsia, using combinations of compounds that alter soluble endoglin, endothelial nitric oxide synthase, PGI₂, TGF-β1, TGF-β3, activin A, BMP2, BMP7, and sFlt-1 expression levels or biological activity. Also disclosed are methods of diagnosing a pregnancy related hypertensive disorder, such as pre-eclampsia and eclampsia, that include the measurement of any one or more of the following: soluble endoglin, endothelial nitric oxide synthase, PGI₂, TGF-β1, TGF-β3, activin A, BMP2, BMP7, and sFlt-1 expression levels or biological activity.

### FIELD OF THE INVENTION

In general, this invention relates to the detection and treatment of subjects having a pregnancy related hypertensive disorder.

### BACKGROUND OF THE INVENTION

Pre-eclampsia is a syndrome of hypertension, edema, and proteinuria that affects 5 to 10% of pregnancies and results in substantial maternal and fetal morbidity and mortality. Pre-eclampsia accounts for at least 200,000 maternal deaths worldwide per year. The symptoms of pre-eclampsia typically appear after the 20^{th} week of pregnancy and are usually detected by routine measuring of the woman's blood pressure and urine. However, these monitoring methods are ineffective for diagnosis of the syndrome at an early stage, which could reduce the risk to the subject or developing fetus, if an effective treatment were available.

Currently there are no known cures for pre-eclampsia. Pre-eclampsia can vary in severity from mild to life threatening. A mild form of pre-eclampsia can be treated with bed rest and frequent monitoring. For moderate to severe cases, hospitalization is recommended and blood pressure medication or anticonvulsant medications to prevent seizures are prescribed. If the condition becomes life threatening to the mother or the baby the pregnancy is terminated and the baby is delivered pre-term.

The proper development of the fetus and the placenta is mediated by several growth factors or angiogenic factors. One of these angiogenic factors is endoglin, also known as CD105. Endoglin is a homodimeric cell membrane glycoprotein that is predominantly expressed on endothelial cells such as syncytiotrophoblasts, human unbilical vein endothelial cells (HUVEC), and on vascular endothelial cells. Endoglin shares sequence identity with betaglycan, a transforming growth factor (TGF)-β receptor (TβR) type III. Endoglin has been shown to be a regulatory component of the TGF-β receptor complex, which modulates angiogenesis, proliferation, differentiation, and apoptosis. Endoglin also binds several other members of the TGF-β superfamily including activin-A, bone morphogenic protein (BMP)-2 and BMP-7. In particular, endoglin binds TGF-β1 and TGF-β3 with high affinity and forms heterotrimeric associations with the TGF-β signaling receptors types I and II. Mutations in the coding region of the endoglin gene are responsible for haemorrhagic telangiectasia type 1 (HHT1), a dominantly inherited vascular disorder characterized by multisystemic vascular dysplasia and recurrent hemorrhage. While endoglin immunoreactivity has been previously detected at increased levels in the plasma of patients with metastatic breast and colorectal cancer, its biochemical characteristics have not been determined and its exact functional role in the pathogenesis of cancer is unclear. Soluble endoglin production has not been reported to be associated with pre-eclampsia or normal pregnancy.

Several factors have been reported to have an association with fetal and placental development and, more specifically, with pre-eclampsia. They include vascular endothelial growth factor (VEGF), soluble Flt-1 receptor (sFlt-1), and placental growth factor (PlGF). VEGF is an endothelial cell-specific mitogen, an angiogenic inducer, and a mediator of vascular permeability. VEGF has also been shown to be important for glomerular capillary repair. VEGF binds as a homodimer to one of two homologous membrane-spanning tyrosine kinase receptors, the fms-like tyrosine kinase (Flt-1) and the kinase domain receptor (KDR), which are differentially expressed in endothelial cells obtained from many different tissues. Flt-1, but not KDR, is highly expressed by trophoblast cells which contribute to placental formation. PlGF is a VEGF family member that is also involved in placental development. PlGF is expressed by cytotrophoblasts and syncytiotrophoblasts and is capable of inducing proliferation, migration, and activation of endothelial cells. PlGF binds as a homodimer to the Flt-1 receptor, but not the KDR receptor. Both PlGF and VEGF contribute to the mitogenic activity and angiogenesis that are critical for the developing placenta.

sFlt-1, which lacks the transmembrane and cytoplasmic domains of the receptor, was recently identified in a cultured medium of human umbilical vein endothelial cells and in vivo expression was subsequently demonstrated in placental tissue. sFlt-1 binds to VEGF with a high affinity but does not stimulate mitogenesis of endothelial cells. Careful regulation of angiogenic and mitogenic signaling pathways is critical for maintaining appropriate proliferation, migration, and angiogenesis by trophoblast cells in the developing placenta.

There is a need for methods of accurately diagnosing subjects at risk for or having pre-eclampsia or eclampsia, particularly before the onset of the most severe symptoms. A treatment is also needed.

### SUMMARY OF THE INVENTION

We have discovered methods for diagnosing and treating pregnancy related hypertensive disorders, including pre-eclampsia and eclampsia.

Using gene expression analysis, we have discovered that levels of soluble endoglin (sEng) are markedly elevated in placental tissue samples from pregnant women suffering from pregnancy complications associated with hypertension, including pre-eclampsia. Using western blotting, we have also discovered that soluble endoglin protein levels are elevated in blood serum samples taken from women with a pregnancy related hypertensive disorder, such as pre-eclampsia or eclampsia. Soluble endoglin may be formed by cleavage of the extracellular portion of the membrane bounds form by proteolytic enzymes. We have discovered that the soluble endoglin detected in these samples contains a minimum of the first 381 amino acids (excluding the leader peptide, 406 including the leader peptide) of the amino terminal portion of the full-length endoglin. Excess soluble endoglin in pre-eclampsia may be depleting the placenta of necessary amounts of these essential angiogenic and mitogenic factors by preventing binding of TGF-β1 to TβRII on endothelial cells leading to decreased signaling, as described herein. We have also discovered that soluble endoglin interferes with TGF-β1 signaling and endothelial nitric oxide synthase (eNOS) activation in endothelial cells, thereby disrupting key homeostatic mechanisms necessary for maintenance of vascular health. We demonstrate that soluble endoglin prevents binding of TGF-β1 to TβRII on endothelial cells leading to decreased signaling. Since circulating TGF-β1 is complexed with latency associated peptide and latent TGF-β1 binding protein, it cannot bind its receptors, unless activated. It is therefore likely that soluble endoglin only inhibits TGF-β1 effects locally where active TGF-β1 is generated. Taken together, these data suggest a crucial role for endoglin in linking TGF-β receptor activation to nitric oxide (NO) synthesis. In addition, our functional studies suggest that soluble endoglin and sFlt1 act in concert to induce vascular damage and HELLP syndrome by interfering with TGF-β1 and VEGF signaling respectively, likely via inhibition of the downstream activation of NOS.

In the present invention, compounds that bind to or neutralize soluble endoglin are used to reduce the elevated levels of soluble endoglin and to treat pregnancy complications associated with hypertension, including pre-eclampsia or eclampsia. For example, antibodies directed to soluble endoglin as well as RNA interference and antisense nucleobase oligomers directed to lowering the levels of biologically active soluble endoglin are also provided. The invention also features the use of any compound (e.g., polypeptide, small molecule, antibody, nucleic acid, and mimetic) that decreases soluble endoglin levels or biological activity or that increases the level or biological activity of a soluble endoglin binding protein (e.g., soluble endoglin binding protein (e.g., TGF-β1, TGF-β3, activin A, Bone Morphogenic Protein (BMP)-2 and BMP-7), NOS, and prostacyclin (PGI₂) either alone or in combination with each other or with any compound that decreases the level of sFlt-1 or increases the level or activity of VEGF or PlGF (see for example, U.S. Patent Application Publication Numbers 20040126828, 20050025762, and 20050170444 and PCT Publication Numbers WO 2004/008946 and WO 2005/077007) to treat or prevent pregnancy related hypertensive disorders, such as pre-eclampsia or eclampsia in a subject. The invention also features methods for measuring levels of soluble endoglin, either alone or in combination with sFlt-1, VEGF, PlGF, TGF-β, eNOS, or PGI₂, as a detection tool for early diagnosis and management of a pregnancy related hypertensive disorder, including pre-eclampsia and eclampsia.

Accordingly, in a first aspect, the invention features a method of treating or preventing a pregnancy related hypertensive disorder in a subject, that includes administering to the subject (i) a compound capable of decreasing soluble endoglin expression levels or biological activity and (ii) a compound capable of decreasing sFlt-1 expression levels or biological activity, for a time and in an amount sufficient to treat or prevent the pregnancy related hypertensive disorder. Pregnancy related hypertensive disorder include, for example, pre-eclampsia, eclampsia, gestational hypertension, chronic hypertension, HELLP syndrome, and pregnancy with a small for gestational age (SGA) infant. Preferably, the pregnancy related hypertensive disorder is pre-eclampsia or eclampsia.

Assays for soluble endoglin or sFlt-1 expression levels or biological activity are known in the art. Preferred compounds will decrease soluble endoglin or sFlt-1 expression levels or biological activity by at least 10%, 20%, 30%, 40%, 50%, 60%, 70%, 80%, 90%, or more. Non-limiting examples of compounds capable of decreasing soluble endoglin expression levels or biological activity include any compound that specifically binds soluble endoglin, for example, a purified soluble endoglin antibody, a soluble endoglin antigen-binding fragment, or a soluble endoglin binding protein (e.g., TGF-β1, TGF-β3, activin A, BMP-2 and BMP-7).

Additional examples of a compound capable of decreasing soluble endoglin expression levels or biological activity include any compound that inhibits a proteolytic enzyme (e.g., a matrix metalloproteinase (P), cathepsin, and elastase) or a compound that increases the level of a growth factor capable of binding to soluble endoglin. Growth factors such as TGF-β1, TGF-β3, activin A, BMP-2, BMP-7, or fragments thereof, are examples of compounds that increases the level of a growth factor capable of binding to soluble endoglin as are cyclosporine, alpha tocopherol, methysergide, bromocriptine, and aldomet.

Non-limiting examples of a compound capable of decreasing sFlt-1 expression levels or biological activity include a compound capable of specifically binding to sFlt-1, such as a purified sFlt-1 antibody or an sFlt-1 antigen-binding fragment; compounds that increase the level of a growth factor capable of binding to sFlt-1, such as nicotine, theophylline, adenosine, nifedipine, minoxidil, and magnesium sulfate, VEGF (e.g., VEGF121, VEGF165, or a modified form of VEGF), PlGF, or fragments thereof.

In preferred embodiments of the above method, a compound capable of decreasing soluble endoglin expression levels or biological activity or a compound capable of decreasing sFlt-1 expression levels or biological activity, or both, can also increase nitric oxide synthase (NOS) activity by at least 10%, 20%, 30%, 40%, 50%, 60%, 70%, 80%, 90%, or more. Assays for NOS activity are known in the art and described herein.

In a second aspect, the invention features a method of treating or preventing a pregnancy related hypertensive disorder in a subject, that includes the step of administering to the subject a compound capable of increasing the expression level or biological activity of NOS, for a time and in an amount sufficient to treat or prevent the pregnancy related hypertensive disorder in the subject. Desirably, the NOS is eNOS. In one embodiment, the compound is a compound that increases the phosphorylation of Ser 1177 of eNOS, such as VEGF (e.g., VEGF121, VEGF165, or a modified form of VEGF), or biologically active fragments thereof, or PlGF or biologically active fragments thereof. In another embodiment, the compound is a compound that increases the dephosphorylation of Thr495 of eNOS, such as TGF-β1 or TGF-β3, activin A, BMP-2, and BMP-7. In another embodiment, the compound is a compound that prevents a reduction in the levels of eNOS or increases the stability of eNOS.

Optionally, the method further includes administering to the subject a compound capable of reducing soluble endoglin expression levels or biological activity, wherein the administering is sufficient to treat or prevent the pregnancy related hypertensive disorder in the subject. Non-limiting examples of a compound capable of reducing soluble endoglin expression or biological activity include a purified antibody that specifically binds soluble endoglin or a soluble endoglin antigen-binding fragment or a compound that inhibits a proteolytic enzyme selected from the group consisting of a matrix metalloproteinase (MMP), cathepsin, and elastase, or growth factors such as TGF-β1, TGF-β3, activin A, BMP-2, BMP-7, or fragments thereof. Exemplary antibodies that specifically bind soluble endoglin include antibodies that bind to a soluble endoglin polypeptide that includes an amino acid sequence selected from the group consisting of amino acids 26 to 437, 40 to 406, or 26 to 587 of the human endoglin sequence shown in FIG. 30B. Additional exemplary antibodies useful in the methods of the invention include an antibody that binds to an epitope on human endoglin that includes amino acids 40 to 86, 144 to 199, 206 to 222, 289 to 304, or 375 to 381 of the human endoglin sequence shown in FIG. 30B.

In one embodiment, the pregnancy related hypertensive disorder is characterized by elevated levels of sFlt-1 polypeptide as compared to a normal reference. Optionally, the method further includes administering to the subject a compound capable of reducing sFlt-1 expression or biological activity, wherein the administering is sufficient to treat or prevent the pregnancy related hypertensive disorder in the subject. Non-limiting examples of a compound capable of reducing soluble sFlt-1 expression or biological activity include a purified antibody that specifically binds sFlt-1 or a sFlt-1 antigen binding fragment, or a growth factor such as VEGF (e.g., VEGF121, VEGF165, or a modified form of VEGF), PlGF, or fragments thereof that bind to sFlt-1.

For any of the above methods, the method can further include the step of administering to a subject an anti-hypertensive compound. In preferred embodiments of any of the above methods, the subject is a pregnant human, a postpartum human, or a non-human (e.g, a cow, a horse, a sheep, a pig, a goat, a dog, and a cat).

For any of the above methods, the method can further include the step of monitoring the pregnancy related hypertensive disorder in the subject, wherein the monitoring includes measuring the level of soluble endoglin polypeptide in a serum or plasma sample from the subject. If the absolute level of soluble endoglin is determined, a level of soluble endoglin polypeptide less than 25 ng/ml indicates an improvement in the pregnancy related hypertensive disorder. Alternatively or additionally, the soluble endoglin level can be measured on two or more occasions or compared to a positive reference sample (e.g., from a subject suffering from a pregnancy related hypertensive disorder), where a decrease in the soluble endoglin level either between measurements or as compared to the positive reference is an indicator of an improvement in the pregnancy related hypertensive disorder. The measuring of the soluble endoglin levels can include the use of an immunological assay. The soluble endoglin can include free, bound, or total soluble endoglin or the level of an endoglin polypeptide resulting from degradation or enzymatic cleavage. The monitoring methods can also include any of the metrics described herein for the diagnosis of pre-eclampsia or eclampsia. For example, the monitoring method can include measuring the levels of sFlt-1 and soluble endoglin in the first and second trimesters in a subject and calculating the delta value of sFlt1×soluble endoglin (sEng) in each trimester using the following equation: [dproduct=(sFlt1×sEng) in the second trimester−(sFlt1×sEng) in the first trimester], where a value greater than 0, 1, 2, or more, including fractions thereof, (e.g., a positive value) is a diagnostic indicator of pre-eclampsia or eclampsia. A decrease in the value over time indicates an improvement in the pregnancy related hypertensive disorder. In another example, the delta product for the sFlt-1 level (dsFlt-1) or the seng level (dsEng) between the first and second trimesters is calculated, and a value greater than 0, 1, 2, or more, including fractions thereof, (e.g., a positive value) for (dsFlt-1) or (dsEng) is a diagnostic indicator of pre-eclampsia or eclampsia. A decrease in the value over time indicates an improvement in the pregnancy related hypertensive disorder.

For any of the monitoring methods, the method can be used to determine the therapeutic dosage of the compound. The method can also further include measuring the level of at least one of sFlt-1, VEGF, or PlGF polypeptide in a sample from the subject and a relationship between these levels can also, but need not, be calculated using a metric. Exemplary metrics include [(sFlt-1+0.25 soluble endoglin)/PlGF], [(soluble endoglin+sFlt-1)/PlGF], [sFlt-1×soluble endoglin], and the dsFlt-1, dsEng, and [dproduct=(sFlt1×sEng) in the second trimester−(sFlt1×sEng) in the first trimester], as described above.

In another aspect, the invention features an antibody or antigen-binding fragment thereof that specifically binds a soluble endoglin polypeptide, wherein the antibody binds to an epitope on human endoglin that includes amino acids 40 to 86, 144 to 199, 206 to 222, 289 to 304, or 375 to 381 of the human endoglin sequence shown in FIG. 30B. In one embodiment, the antibody or antigen-binding fragment prevents binding of a growth factor (e.g., TGF-β1, TGF-β3, activin A, BMP-2, and BMP-7) to soluble endoglin.

The antibody can be any type of antibody or antibody fragment including a monoclonal antibody, chimeric antibody, humanized antibody, human antibody, an antibody lacks an Fc portion, is an F(ab′)₂, an Fab, or an Fv structure. In one embodiment, the antibody or antigen-binding fragment thereof is present in a pharmaceutically acceptable carrier.

In another aspect, the invention features a method of diagnosing a subject as having, or having a predisposition to, a pregnancy related hypertensive disorder, that includes measuring the level of a soluble endoglin polypeptide and at least one additional polypeptide selected from the group consisting of soluble endoglin binding proteins (e.g., TGF-β1, TGF-β3, activin A, BMP2, and BMP 7) and downstream mediators of soluble endoglin signaling (e.g., eNOS and PGI₂) in a sample from the subject, wherein an increase in the soluble endoglin level and a decrease in the level of the at least one additional polypeptide as compared to a normal reference sample, standard, or level is a diagnostic indicator of a pregnancy related hypertensive disorder or a propensity to develop a pregnancy related hypertensive disorder.

In another aspect, the invention features a method of diagnosing a subject as having, or having a predisposition to, a pregnancy related hypertensive disorder, that includes measuring the level of a soluble endoglin polypeptide and an sFlt-1 polypeptide from the subject and calculating the relationship between the levels of soluble endoglin and sFlt-1 using a [soluble endoglin×sFlt-1] metric, wherein an increase in the metric in the subject sample relative to a normal reference sample, is a diagnostic indicator of a pregnancy related hypertensive disorder in the subject.

In another aspect, the invention features a method of diagnosing a subject as having, or having a predisposition to, a pregnancy related hypertensive disorder, that includes measuring the levels of sFlt-1 and soluble endoglin in the first and second trimesters in a subject and calculating the delta value of sFlt1×soluble endoglin (seng) in each trimester using the following equation: [dproduct=(sFlt1×sEng) in the second trimester−(sFlt1×sEng) in the first trimester], where a value greater than 0, 1, 2, or more, including fractions thereof (e.g., a positive value) is a diagnostic indicator of pre-eclampsia or eclampsia. A positive value can also be an indicator of pre-term pre-eclampsia. Such a measurement can be taken on numerous occasions during the first and second trimesters and the dproduct can be followed over time.

In another aspect, the invention features a method of diagnosing a subject as having, or having a predisposition to, a pregnancy related hypertensive disorder, that includes measuring the levels of sFlt-1 and soluble endoglin in the first and second trimesters in a subject and calculating the delta product for the sFlt-1 level (dsFlt-1) or the sEng level (dsEng) between the first and second trimesters, where a value greater than 0, 1, 2, or more, including fractions thereof, (e.g., a positive value) for (dsFlt-1) or (dsEng) is a diagnostic indicator of pre-eclampsia or eclampsia.

For any of the diagnostic methods of the invention, the measuring can include the use of an immunological assay, such as an ELISA. In one embodiment, the normal reference sample is a prior sample from the subject. In another embodiment, the metric also includes the body mass index of the mother or the gestational age of the fetus. The sample can be a bodily fluid (e.g., urine, amniotic fluid, blood, serum, and plasma), cell (e.g., an endothelial cell, a leukocyte, a monocyte, and a cell derived from the placenta), or a tissue (e.g., placental tissue) of the subject in which the soluble endoglin is normally detectable. The subject can be a non-pregnant human, a pregnant human, a postpartum human, or a non-human (e.g., a cow, a horse, a sheep, a pig, a goat, a dog, or a cat) and the method can be used to diagnose a pregnancy related hypertensive disorder or a propensity to develop a pregnancy related hypertensive disorder (e.g., at least four weeks prior to the onset of symptoms).

In yet another aspect, the invention features a kit for the diagnosis of a pregnancy related hypertensive disorder in a subject that includes (i) a soluble endoglin binding agent and (ii) at least one additional binding agent that binds to a polypeptide selected from the group consisting of TGF-β1, TGF-β3, eNOS, and PGI₂ and (iii) instructions for the use of the binding agent of (i) and the at least one binding agent of (ii) for the diagnosis of a pregnancy related hypertensive disorder or a propensity to develop a pregnancy related hypertensive disorder. The binding agents can be an antibody, or antigen-binding fragment thereof, that specifically binds soluble endoglin or antibody, or antigen binding fragment thereof, that specifically binds TGF-β1, TGF-β3, eNOS, or PGI₂.

Optionally, the kit can also include a VEGF, sFlt-1, or PlGF binding molecule.

In another aspect, the invention features a method of identifying a compound that ameliorates a pregnancy related hypertensive disorder, that includes the following steps:

(a) contacting a cell with a soluble endoglin compound;

(b) determining the phosphorylation state of Thr495 of eNOS in the cell after contacting with the soluble endoglin compound;

(c) contacting the cell with a candidate compound;

(d) determining the phosphorylation state of Thr495 of eNOS in the cell after contacting the cell with the candidate compound; and

(e) comparing the phosphorylation state determined in step (b) and step (d), wherein an increase in the dephosphorylation of Thr 495 of eNOS in step (d) as compared to step (b) identifies the candidate compound as a compound that ameliorates a pregnancy related hypertensive disorder.

In yet another aspect, the invention features a method of identifying a compound that ameliorates a pregnancy related hypertensive disorder, that includes the following steps:

(a) contacting a cell with a Smad2/3 dependent reporter construct and a soluble endoglin compound;

(b) determining the level of activation of the Smad2/3 reporter construct in the cell of step (a);

(c) contacting the cell of step (a) with a candidate compound;

(d) determining the level of activation of the Smad2/3 reporter construct in the cell of step (c); and

(e) comparing the level of activation of the Smad2/3 reporter construct determined in step (b) and step (d),

wherein an increase in the level of activation of the Smad2/3 reporter construct in step (d) as compared to step (b) identifies the candidate compound as a compound that ameliorates a pregnancy related hypertensive disorder.

For any of the above aspects, the pregnancy related hypertensive disorder can be pre-eclampsia, eclampsia, gestational hypertension, chronic hypertension, HELLP syndrome, and pregnancy with a SGA infant. In one embodiment, the pregnancy related hypertensive disorder is pre-eclampsia or eclampsia.

As described below, we have discovered that deregulation of both the soluble endoglin/TGF-β and the sFlt-1/VEGF/PlGF signaling pathways can act together to further the pathology of the pregnancy related hypertensive disorder. Therefore, the invention also features combinations of the methods described herein with any of the therapeutic, diagnostic, or monitoring methods described in U.S. Patent Application Publication Numbers 20040126828, 20050025762, 20050170444, 2006/0067937, and 20070104707 and PCT Publication Numbers WO 2004/008946, WO 2005/077007, and WO 06/034507.

For the purpose of the present invention, the following abbreviations and terms are defined below.

By “alteration” is meant a change (increase or decrease). An alteration can include a change in the expression levels of a gene or polypeptide as detected by standard art known methods such as those described below. As used herein, an alteration includes a 10% change in expression levels, preferably a 25% change, more preferably a 40%, 50%, 60%, 70%, 80%, 90% or greater change in expression levels. “Alteration” can also indicate a change (increase or decrease) in the biological activity of any of the polypeptides of the invention (e.g., soluble endoglin, sFlt-1, VEGF, PlGF, eNOS, or TGFβ family member). As used herein, an alteration includes a 10% change in biological activity, preferably a 25% change, more preferably a 40%, 50%, 60%, 70%, 80%, 90% or greater change in biological activity. Examples of biological activity for soluble endoglin are angiogenesis and binding to substrates such as activin-A, BMP 2, BMP-7, TGF-β1 and TGF-β3. The biological activity of soluble endoglin can be measured by ligand binding assays, immunoassays, and angiogenesis assays that are standard in the art or are described herein. An example of such an assay is the in vitro matrigel endothelial tube formation assay in which antagonism of endoglin signaling led to massive loss of capillary formation (Li et al., Faseb Journal 14:55-64 (2000)). Examples of biological activity for eNOS are known in the art and include catalyzing the formation of nitric oxide or “NO” from oxygen and arginine. Examples of biological activity for TGF-β include regulation of growth, differentiation, motility, tissue remodeling, neurogenesis, wound repair, apoptosis, and angiogenesis in many cell types. Such activities can be measured by assays known in the art or described herein. TGF-β also inhibits cell proliferation in many cell types and can stimulate the synthesis of matrix proteins. Other examples of biological activity for PlGF or VEGF include binding to receptors as measured by immunoassays, ligand binding assays or Scatchard plot analysis, and induction of cell proliferation or migration as measured by BrdU labeling, cell counting experiments, or quantitative assays for DNA synthesis such as ³H-thymidine incorporation. Examples of biological activity for sFlt-1 include binding to PlGF and VEGF as measured by immunoassays, ligand binding assays, or Scatchard plot analysis. Additional examples of assays for biological activity for each of the polypeptides are described herein.

By “antisense nucleobase oligomer” is meant a nucleobase oligomer, regardless of length, that is complementary to the coding strand or mRNA of an endoglin gene. By a “nucleobase oligomer” is meant a compound that includes a chain of at least eight nucleobases, preferably at least twelve, and most preferably at least sixteen bases, joined together by linkage groups. Included in this definition are natural and non-natural oligonucleotides, both modified and unmodified, as well as oligonucleotide mimetics such as Protein Nucleic Acids, locked nucleic acids, and arabinonucleic acids. Numerous nucleobases and linkage groups may be employed in the nucleobase oligomers of the invention, including those described in U.S. Patent Publication Nos. 20030114412 (see for example paragraphs 27-45 of the publication) and 20030114407 (see for example paragraphs 35-52 of the publication), incorporated herein by reference. The nucleobase oligomer can also be targeted to the translational start and stop sites. Preferably the antisense nucleobase oligomer comprises from about 8 to 30 nucleotides. The antisense nucleobase oligomer can also contain at least 40, 60, 85, 120, or more consecutive nucleotides that are complementary to endoglin mRNA or DNA, and may be as long as the full-length mRNA or gene.

By “binding” is meant a non-covalent or a covalent interaction, preferably non-covalent, that holds two molecules together. For example, two such molecules could be a ligand and its receptor, an enzyme and an inhibitor of that enzyme, an enzyme and its substrate, or an antibody and an antigen. Non-covalent interactions include, but are not limited to, hydrogen bonding, ionic interactions among charged groups, van der Waals interactions, and hydrophobic interactions among non-polar groups. One or more of these interactions can mediate the binding of two molecules to each other. Binding may exhibit discriminatory properties such as specificity or selectivity.

By “body mass index” is meant a number, derived by using height and weight measurements, that gives a general indication of whether or not weight falls within a healthy range. The formula generally used to determine the body mass index is a person's weight in kilograms divided by a person's height in meters squared or weight (kg)/(height (m))².

By “compound” is meant any small molecule chemical compound (peptidyl or non-peptidyl), antibody, nucleic acid molecule, polypeptide, or fragments thereof. Compounds particularly useful for the therapeutic methods of the invention can alter, preferably decrease, the levels or biological activity of soluble endoglin by at least 10%, 20%, 30%, 40%, 50%, 60%, 70%, 80%, 90% or more.

By “chimeric antibody” is meant a polypeptide comprising at least the antigen-binding portion of an antibody molecule linked to at least part of another protein (typically an immunoglobulin constant domain).

By “double-stranded RNA (dsRNA)” is meant a ribonucleic acid molecule comprised of both a sense and an anti-sense strand. dsRNAs are typically used to mediate RNA interference.

By “endoglin” or “Eng,” also known as CD105, is meant a mammalian growth factor that has endoglin biological activity (see Fonsatti et al., Oncogene 22:6557-6563, 2003; Fonsatti et al., Curr. Cancer Drug Targets 3:427-432, 2003; and Cheifetz et al., J. Biol. Chem. 267:19027-19030 (1992)) and is homologous to the protein defined by any of the following GenBank accession numbers: AAH29080 and NP_{—}031958 (mouse); AAS67893 (rat); NP_{—}000109, P17813, VSP_{—}004233, CAA80673 (pig); and CAA50891 and AAC63386 (human), or described in U.S. Pat. No. 6,562,957. Endoglin is a homodimeric cell membrane glycoprotein which is expressed at high levels in proliferating vascular endothelial cells and in the syncytiotrophoblasts from placentas. There are two distinct isoforms of endoglin, L and S, which differ in their cytoplasmic tails by 47 amino acids. Both isoforms are included in the term endoglin as used herein. Endoglin binds to TGF-β family members and, in the presence of TGF-β, endoglin can associate with the TGF-β signaling receptors RI and RII, and potentiate the response to the growth factors. Endoglin biological activities include binding to substrates such as TGF-β family members such as activin-A, BMP 2, BMP-7, TGF-β1 and TGF-β3; induction of angiogenesis, regulation of cell proliferation, attachment, migration, invasion; and activation of endothelial cells. Assays for endoglin biological activities are known in the art and include ligand binding assays or Scatchard plot analysis; BrdU labeling, cell counting experiments, or quantitative assays for DNA synthesis such as ³H-thymidine incorporation used to measure cell proliferation; and angiogenesis assays such as those described herein or in McCarty et al., Intl. J. Oncol. 21:5-10, 2002; Akhtar et al. Clin. Chem. 49:3240, 2003; and Yamashita et al, J. Biol. Chem. 269:1995-2001, 1994).

By “soluble endoglin polypeptide” or “sEng” is meant any circulating, non-membrane bound form of endoglin which includes at least a part of the extracellular portion of the endoglin protein and is substantially identical (e.g., 60%, 70%, 80%, 90%, 995%, 96%, 97%, 98%, 99%, or 100%) to the amino acid sequence encoding the extracellular portion of the endoglin protein (see FIGS. 1 and 2B). Soluble endoglin can result from the cleavage of the membrane bound form of endoglin by a proteolytic enzyme. One potential cleavage site is at amino acid 437 of human endoglin producing a soluble endoglin polypeptide that includes amino acids 1-437 of the endoglin polypeptide, including the peptide leader sequence, which is typically cleaved off in the ER (see FIGS. 3A and 3B), or a protein that is substantially identical to amino acids 1-437 of the endoglin polypeptide. Additional forms of soluble endoglin contemplated by the invention include a protein substantially identical to amino acids 40 (glycine) to 406 (arginine) of the human endoglin shown in FIG. 30B, substantially identical to amino acids 1 to 587 of human endoglin (the entire extracellular domain, including the peptide leader sequence, commercially available from R&D Systems, catalog number 1097-EN), substantially identical to amino acids 40 to 587 of human endoglin shown in FIG. 30B (this is the entire extracellular domain with the peptide leader sequence excluded), any polypeptide that includes the peptides identified in bold and underlined in FIG. 30B, and any polypeptide that includes the regions or domains of soluble endoglin that are required for binding to TGF-β or TGF-β receptors. It should be noted that the numbering of both endoglin and soluble endoglin depends on whether the leader peptide sequence is included. The numbering of endoglin shown in FIG. 30B, starts at amino acid 26 (where the absent leader peptide sequence would be amino acids 1-25). Soluble endoglin can also include circulating degradation products or fragments that result from enzymatic cleavage of endoglin and that maintain endoglin biological activity. Preferred soluble endoglin polypeptides have soluble endoglin biological activity such as binding to substrates such as TGF-β family members or TGF-β receptors, inhibiting the biological activity of TGF-β family members, or reversing or inhibiting angiogenesis by at least 10%, 20%, 30%, 40%, 50%, 60%, 70%, 80%, 90%, or more. Examples of assays for measuring these activities are known in the art and described in U.S. Patent Application Publication Nos. 20060067937, 20050267021, and 20070104707 and PCT Publication No. WO 06/034507, incorporated herein by reference. For example, soluble endoglin biological activity can include the ability to reverse, reduce, or inhibit angiogenesis induced by TGF-β or the ability to reverse activation of Smad 2/3 or Smad 2/3 dependent transcriptional activation. Soluble endoglin polypeptides may be isolated from a variety of sources, such as from mammalian tissue or cells (e.g., placental tissue or cells), or prepared by recombinant or synthetic methods. The term soluble endoglin also encompasses modifications to the polypeptide, fragments, derivatives, analogs, and variants of the endoglin polypeptide, examples of which are described below.

By “endoglin nucleic acid” is meant a nucleic acid that encodes any of the endoglin proteins described above. For example, the gene for human endoglin consists of 14 exons, where exon 1 encodes the signal peptide sequence, exons 2-12 encode the extracellular domain (includes exon 9a and 9b), exon 13 encodes the transmembrane domain, and exon 14 encodes C-terminal cytoplasmic domain (see FIGS. 1, 2A, and 2B). Desirably, the endoglin nucleic acid encodes any of the soluble endoglin polypeptides described above or is substantially identical (60%, 65%, 70%, 75%, 80%, 85%, 90%, 95%, 96%, 97%, 98%, 99%, or 100%) to the nucleic acid sequence set forth in FIG. 2A. It should be noted that the circulating protein is predicted to lack the peptide leader sequence (amino acids 1-25).

By “epitope” is meant a sequence of amino acids which, either as a result of linear structure or three dimensional conformation, forms the binding site for an antibody.

By “expression” is meant the detection of a gene or polypeptide by standard art known methods. For example, polypeptide expression is often detected by western blotting, DNA expression is often detected by Southern blotting or polymerase chain reaction (PCR), and RNA expression is often detected by northern blotting, PCR, or RNAse protection assays. Methods to measure protein expression levels generally include, but are not limited to: Western blot, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, matrix-assisted laser desorption/ionization time-of-flight MALDI-TOF) mass spectrometry, microcytometry, microarray, microscopy, fluorescence activated cell sorting (FACS), and flow cytometry, as well as assays based on a property of the protein including but not limited to enzymatic activity or interaction with other protein partners. Exemplary assays are described in detail in U.S. Patent Application Publication No. 2006/0067937 and PCT Publication No. WO 06/034507. Any compound that decreases soluble endoglin levels by at least 10%, 20%, preferably 30%, more preferably at least 40% or 50%, and most preferably at least 60%, 70%, 80%, 90% or more is considered a therapeutic compound of the invention.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, 1000, 1250, 1500, 1750, 1800 or more nucleotides or 10, 20, 30, 40, 50, 60, 70, 80, 90, 100, 150, 200, 250, 300, 350, 400, 450, 500, 550, 600, 640 amino acids or more. Exemplary fragments of soluble endoglin include from 1 to 437 amino acids (including the peptide leader sequence), 26 to 437 amino acids (excluding the leader sequence), from 40 to 406 amino acids, or from 1 to 587 amino acids, and from 1 to 1311, 10 to 1311, 80 to 1030, or 1 to 1761 nucleotides.

By “gestational age” is meant a reference to the age of the fetus, counting from the first day of the mother's last menstrual period usually referred to in weeks.

By “gestational hypertension” is meant the development of high blood pressure without proteinuria after 20 weeks of pregnancy.

By a “history of pre-eclampsia or eclampsia” is meant a previous diagnosis of pre-eclampsia or eclampsia or pregnancy induced hypertension in the subject themselves or in a related family member.

By “homologous” is meant any gene or protein sequence that bears at least 30% homology, more preferably 40%, 50%, 60%, 70%, 80%, and most preferably 90% or more homology to a known gene or protein sequence over the length of the comparison sequence. A “homologous” protein can also have at least one biological activity of the comparison protein. In general, for proteins, the length of comparison sequences will be at least 10 amino acids, preferably 20, 30, 40, 50, 60, 70, 80, 90, 100, 110, 120, 130, 140, 150, 200, 250, 300, 350, 400, 437, or at least 587 amino acids or more. For nucleic acids, the length of comparison sequences will generally be at least 25, 50, 100, 125, 150, 200, 250, 300, 350, 400, 450, 500, 550, 600, 650, 700, 800, 900, 1000, 1100, 1200, 1311, or at least 1761 nucleotides or more. “Homology” can also refer to a substantial similarity between an epitope used to generate antibodies and the protein or fragment thereof to which the antibodies are directed. In this case, homology refers to a similarity sufficient to elicit the production of antibodies that can specifically recognize the protein at issue.

By “humanized antibody” is meant an immunoglobulin amino acid sequence variant or fragment thereof that is capable of binding to a predetermined antigen. Ordinarily, the antibody will contain both the light chain as well as at least the variable domain of a heavy chain. The antibody also may include the CH1, hinge, CH2, CH3, or CH4 regions of the heavy chain. The humanized antibody comprises a framework region (FR) having substantially the amino acid sequence of a human immunoglobulin and a complementarity determining region (CDR) having substantially the amino acid sequence of a non-human immunoglobulin (the “import” sequences).

Generally, a humanized antibody has one or more amino acid residues introduced into it from a source that is non-human. In general, the humanized antibody will comprise substantially all of at least one, and typically two, variable domains (Fab, Fab′, F(ab′)₂, Fabc, Fv) in which all or substantially all of the CDR regions correspond to those of a non-human immunoglobulin and all or substantially all of the FR regions are those of a human immunoglobulin consensus sequence. The humanized antibody optimally will comprise at least a portion of an immunoglobulin constant region (Fc), typically that of a human immunoglobulin. By “complementarity determining region (CDR)” is meant the three hypervariable sequences in the variable regions within each of the immunoglobulin light and heavy chains. By “framework region (FR)” is meant the sequences of amino acids located on either side of the three hypervariable sequences (CDR) of the immunoglobulin light and heavy chains.

The FR and CDR regions of the humanized antibody need not correspond precisely to the parental sequences, e.g., the import CDR or the consensus FR may be mutagenized by substitution, insertion or deletion of at least one residue so that the CDR or FR residue at that site does not correspond to either the consensus or the import antibody. Such mutations, however, will not be extensive. Usually, at least 75%, preferably 90%, and most preferably at least 95% of the humanized antibody residues will correspond to those of the parental FR and CDR sequences.

By “hybridize” is meant pair to form a double-stranded molecule between complementary polynucleotide sequences, or portions thereof, under various conditions of stringency. (See, e.g., Wahl and Berger Methods Enzymol. 152:399, 1987; Kimmel, Methods Enzymol. 152:507, 1987.) For example, stringent salt concentration will ordinarily be less than about 750 mM NaCl and 75 mM tri sodium citrate, preferably less than about 500 mM NaCl and 50 mM trisodium citrate, and most preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and most preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a most preferred embodiment, wash steps will occur at 68° C. in 15 mM NaCl, 1.5 mM trisodium citrate, and 0.1% SDS. Additional variations on these conditions will be, readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “intrauterine growth retardation (IUGR)” is meant a syndrome resulting in a birth weight which is less that 10 percent of the predicted fetal weight for the gestational age of the fetus. The current World Health Organization criterion for low birth weight is a weight less than 2,500 gm (5 lbs. 0.8 oz.) or below the 10^{th} percentile for gestational age according to U.S. tables of birth weight for gestational age by race, parity, and infant sex (Zhang and Bowes, Obstet. Gynecol. 86:200-208, 1995). These low birth weight babies are also referred to as “small for gestational age (SGA)”. Pre-eclampsia is a condition known to be associated with IUGR or SGA.

By “metric” is meant a measure. A metric may be used, for example, to compare the levels of a polypeptide or nucleic acid molecule of interest. Exemplary metrics include, but are not limited to, mathematical formulas or algorithms, such as ratios. The metric to be used is that which best discriminates between levels of soluble endoglin, sFlt-1, VEGF, PlGF, or any combination thereof, in a subject having pregnancy related hypertensive disorder, such as pre-eclampsia or eclampsia, and a normal control subject. Depending on the metric that is used the diagnostic indicator of pregnancy related hypertensive disorder may be significantly above or below a reference value (e.g., from a control subject not having a pregnancy related hypertensive disorder). Soluble endoglin level is determined by measuring the amount of free, bound (i.e., bound to growth factor), or total (free+bound) soluble endoglin. sFlt-1 level is measured by measuring the amount of free, bound (i.e., bound to growth factor), or total sFlt-1 (bound+free). VEGF or PlGF levels are determined by measuring the amount of free PlGF or free VEGF (i.e., not bound to sFlt-1). One exemplary metric is [sFlt-1/(VEGF+PlGF)], also referred to as the pre-eclampsia anti-angiogenic index (PAAI). Another example is the following soluble endoglin anti-angiogenic index: (sFlt-1+0.25(soluble endoglin polypeptide))/PlGF. Yet another exemplary metric is the following: (soluble endoglin+sFlt-1)/PlGF. An increase in the value of either of these two exemplary metrics as compared to a normal reference is a diagnostic indicator of pre-eclampsia or eclampsia. Another example includes the measurement of the levels of sFlt-1 and soluble endoglin in the first and second trimesters in a subject and calculating the delta value of sFlt 1×soluble endoglin (sEng) in each trimester using the following equation: [dproduct (sFlt1×sEng) in the second trimester−(sFlt1×sEng) in the first trimester], where a value greater than 0, 1, 2, or more (e.g., a positive value) is a diagnostic indicator of pre-eclampsia or eclampsia. Additional metrics include the dproduct of the sFlt-1 level (dsFlt-1) and the sEng level (dsEng) between the first and second trimesters, where a value greater than 0, 1, 2, or more (e.g., a positive value) for (dsFlt-1) or (dsEng) is a diagnostic indicator of pre-eclampsia or eclampsia.

Any of the metrics of the invention can further include the BMI of the mother or gestational age of the infant, or parity. Any of the metrics can also include eNOS, TGF-β1 or β3 or PGI₂ levels as well.

By “nitric oxide synthase” or “NOS” is meant an enzyme that catalyzes the formation of nitric oxide (NO) from oxygen and arginine. NOS is a complex enzyme containing several cofactors, a heme group which is part of the catalytic site, an N-terminal oxygenase domain, which belongs to the class of haem-thiolate proteins, and a C-terminal reductase domain which is homologous to NADPH:P450 reductase. NOS produces NO by catalysing a five-electron oxidation of a guanidino nitrogen of L-arginine (L-Arg). Oxidation of L-Arg to L-citrulline occurs via two successive monooxygenation reactions producing N-hydroxy-L-arginine as an intermediate. The interdomain linker between the oxygenase and reductase domains contains a CaM-binding sequence. NO functions at low concentrations as a signal in many diverse physiological processes such as blood pressure control, neurotransmission, learning and memory, and at high concentrations as a defensive cytotoxin.

In mammals, three distinct genes encode NOS isozymes: neuronal (nNOS or NOS-1), cytokine-inducible (iNOS or NOS-2) and endothelial (eNOS or NOS-3). eNOS is membrane associated and eNOS localization to endothelial membranes is mediated by cotranslational N-terminal myristoylation and post-translational palmitoylation. In preferred embodiments of the invention, the NOS is eNOS.

By “pre-eclampsia anti-angiogenesis index (PAAI)” is meant the ratio of sFlt-1/VEGF+PlGF used as an indicator of anti-angiogenic activity. A PAAI greater than 10, more preferably greater than 20, is indicative of a pregnancy related hypertensive disorder, such as pre-eclampsia or risk of pre-eclampsia.

By “soluble endoglin anti-angiogenic index” is meant the ratio of (sFlt-1+0.25 soluble endoglin)/PlGF. For example, a value of 75, or higher, preferably 100 or higher, or more preferably 200 or higher is indicative of a pregnancy complication associated with hypertension, such as pre-eclampsia or eclampsia.

By “operably linked” is meant that a gene and a regulatory sequence(s) are connected in such a way as to permit gene expression when the appropriate molecules (e.g., transcriptional activator proteins) are bound to the regulatory sequence(s).

By “pharmaceutically acceptable carrier” is meant a carrier that is physiologically acceptable to the treated mammal while retaining the therapeutic properties of the compound with which it is administered. One exemplary pharmaceutically acceptable carrier substance is physiological saline. Other physiologically acceptable carriers and their formulations are known to one skilled in the art and described, for example, in Remington's Pharmaceutical Sciences, (20th edition), ed. A. Gennaro, 2000, Lippincott, Williams & Wilkins, Philadelphia, Pa.

By “placental growth factor (PlGF)” is meant a mammalian growth factor that is homologous to the protein defined by GenBank accession number P49763 and that has PlGF biological activity. PlGF is a glycosylated homodimer belonging to the VEGF family and can be found in two distinct isoforms through alternative splicing mechanisms. PlGF is expressed by cyto- and syncytiotrophoblasts in the placenta and PlGF biological activities include induction of proliferation, migration, and activation of endothelial cells, particularly trophoblast cells.

By “polymorphism” is meant a genetic variation, mutation, deletion or addition in a soluble endoglin, sFlt-1, PlGF, or VEGF nucleic acid molecule that is indicative of a predisposition to develop pre-eclampsia or eclampsia. Such polymorphisms are known to the skilled artisan and are described, for example, by Raab et al. (Biochem. J. 339:579-588, 1999) and Parry et al. (Eur. J Immunogenet. 26:321-323, 1999). A polymorphism may be present in the promoter sequence, an open reading frame, intronic sequence, or untranslated 3′ region of a gene. Known examples of such polymorphisms in the endoglin gene include a 6 base insertion of GGGGGA in intron 7 at 26 bases beyond the 3′ end of exon 7 (Ann. Neurol. 41:683-6, 1997).

By “pregnancy related hypertensive disorder” is meant any condition or disease or pregnancy that is associated with or characterized by an increase in blood pressure. Included among these conditions are pre-eclampsia (including premature pre-eclampsia, severe pre-eclampsia), eclampsia, gestational hypertension, HELLP syndrome, (hemolysis, elevated liver enzymes, low platelets), abruption placenta, chronic hypertension, pregnancy with intra uterine growth restriction, and pregnancy with a small for gestational age (SGA) infant. It should be noted that although pregnancy with a SGA infant is not often associated with hypertension, it is included in this definition.

By “pre-eclampsia” is meant the multi-system disorder that is characterized by hypertension with proteinuria or edema, or both, glomerular dysfunction, brain edema, liver edema, or coagulation abnormalities due to pregnancy or the influence of a recent pregnancy. All forms of pre-eclampsia, such as premature, mild, moderate, and severe pre-eclampsia are included in this definition. Pre-eclampsia generally occurs after the 20^{th} week of gestation. Pre-eclampsia is generally defined as some combination of the following symptoms: (1) a systolic blood pressure (BP) >140 mmHg and a diastolic BP>90 mmHg after 20 weeks gestation (generally measured on two occasions, 4-168 hours apart), (2) new onset proteinuria (1+ by dipstik on urinalysis, >300 mg of protein in a 24-hour urine collection, or a single random urine sample having a protein/creatinine ratio >0.3), and (3) resolution of hypertension and proteinuria by 12 weeks postpartum. Severe pre-eclampsia is generally defined as (1) a diastolic BP>110 mmHg (generally measured on two occasions, 4-168 hours apart) or (2) proteinuria characterized by a measurement of 3.5 grams or more protein in a 24-hour urine collection or two random urine specimens with at least 3+ protein by dipstick. In pre-eclampsia, hypertension and proteinuria generally occur within seven days of each other. In severe pre-eclampsia, severe hypertension, severe proteinuria and HELLP syndrome (hemolysis, elevated liver enzymes, low platelets) or eclampsia can occur simultaneously or only one symptom at a time. HELLP syndrome is characterized by evidence of thrombocytopenia (<100000 cells/μl), increased LDH (>600 IU/L) and increased AST (>70 IU/L). Occasionally, severe pre-eclampsia can lead to the development of seizures. This severe form of the syndrome is referred to as “eclampsia.” Eclampsia can also include dysfunction or damage to several organs or tissues such as the liver (e.g., hepatocellular damage, periportal necrosis) and the central nervous system (e.g., cerebral edema and cerebral hemorrhage). The etiology of the seizures is thought to be secondary to the development of cerebral edema and focal spasm of small blood vessels in the kidney.

By “premature pre-eclampsia” is meant pre-eclampsia with onset of symptoms <37 weeks or <34 weeks.

By “prostacyclin” or “PGI₂” is meant a member of the family of lipid molecules known as eicosanoids. It is produced in endothelial cells from prostaglandin H2 (PGH2) by the action of the enzyme prostacyclin synthase and is mainly synthesized by vascular endothelium and smooth muscle. PGI₂ biological activity includes inhibition of platelet aggregation, relaxation of smooth muscle, reduction of systemic and pulmonary vascular resistance by direct vasodilation, and natriuresis in kidney.

By “protein” or “polypeptide” or “polypeptide fragment” is meant any chain of more than two amino acids, regardless of post-translational modification (e.g., glycosylation or phosphorylation), constituting all or part of a naturally occurring polypeptide or peptide, or constituting a non-naturally occurring polypeptide or peptide.

By “reference sample” is meant any sample, standard, or level that is used for comparison purposes. A “normal reference sample” can be a prior sample taken from the same subject, a sample from a pregnant subject not having a pregnancy related hypertensive disorder, such as pre-eclampsia or eclampsia, a subject that is pregnant but the sample was taken early in pregnancy (e.g., in the first or second trimester or before the detection of a pregnancy related hypertensive disorder, such as pre-eclampsia or eclampsia), a subject that is pregnant and has no history of a pregnancy related hypertensive disorder, such as pre-eclampsia or eclampsia, a subject that is not pregnant, a sample of a purified reference polypeptide at a known normal concentration (i.e., not indicative of a pregnancy related hypertensive disorder, such as pre-eclampsia or eclampsia). By “reference standard or level” is meant a value or number derived from a reference sample. A normal reference standard or level can be a value or number derived from a normal subject that is matched to the sample subject by at least one of the following criteria: gestational age of the fetus, maternal age, maternal blood pressure prior to pregnancy, maternal blood pressure during pregnancy, BMI of the mother, weight of the fetus, prior diagnosis of pre-eclampsia or eclampsia, and a family history of pre-eclampsia or eclampsia. A “positive reference” sample, standard or value is a sample or value or number derived from a subject that is known to have a pregnancy related hypertensive disorder, such as pre-eclampsia or eclampsia, that is matched to the sample subject by at least one of the following criteria: gestational age of the fetus, maternal age, maternal blood pressure prior to pregnancy, maternal blood pressure during pregnancy, BMI of the mother, weight of the fetus, prior diagnosis of a pregnancy related hypertensive disorder, and a family history of a pregnancy related hypertensive disorder

By “reduce or inhibit” is meant the ability to cause an overall decrease preferably of 20% or greater, more preferably of 40%, 50%, 60%, 70%, 80%, 90% or greater change in the level of protein or nucleic acid, detected by the aforementioned assays (see “expression”), as compared to an untreated sample

By “sample” is meant a tissue biopsy, cell, bodily fluid (e.g., blood, serum, plasma, urine, saliva, amniotic fluid, or cerebrospinal fluid) or other specimen obtained from a subject. Desirably, the biological sample includes soluble endoglin nucleic acid molecules or polypeptides or both.

By “small interfering RNAs (siRNAs)” is meant an isolated dsRNA molecule, preferably greater than 10 nucleotides (nt) in length, more preferably greater than 15 nucleotides in length, and most preferably greater than 19 nucleotides in length that is used to identify the target gene or mRNA to be degraded. A range of 19-25 nucleotides is the most preferred size for siRNAs. siRNAs can also include short hairpin RNAs in which both strands of an siRNA duplex are included within a single RNA molecule. siRNA includes any form of dsRNA (proteolytically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides. Such alterations can include the addition of non-nucleotide material, such as to the end(s) of the 19, 20, 21, 22, 23, 24, or 25 nt RNA or internally (at one or more nucleotides of the RNA). In a preferred embodiment, the RNA molecules contain a 3′ hydroxyl group. Nucleotides in the RNA molecules of the present invention can also comprise non-standard nucleotides, including non-naturally occurring nucleotides or deoxyribonucleotides. Collectively, all such altered RNAs are referred to as analogs of RNA. siRNAs of the present invention need only be sufficiently similar to natural RNA that it has the ability to mediate RNA interference (RNAi). As used herein, RNAi refers to the ATP-dependent targeted cleavage and degradation of a specific mRNA molecule through the introduction of small interfering RNAs or dsRNAs into a cell or an organism. As used herein “mediate RNAi” refers to the ability to distinguish or identify which RNAs are to be degraded.

By “soluble endoglin binding molecule” is meant a protein or small molecule compound that binds, preferably specifically binds, a soluble endoglin polypeptide. A soluble endoglin binding molecule may be, for example, an antibody, antibody-related peptide, one or more CDR regions of a soluble endoglin binding antibody, or soluble endoglin interacting protein.

By “soluble Flt-1 (sFlt-1)” (also known as sVEGF-R1) is meant the soluble form of the Flt-1 receptor, that is homologous to the protein defined by GenBank accession number U01134, and that has sFlt-1 biological activity. The biological activity of an sFlt-1 polypeptide may be assayed using any standard method, for example, by assaying sFlt-1 binding to VEGF. sFlt-1 lacks the transmembrane domain and the cytoplasmic tyrosine kinase domain of the Flt-1 receptor. sFlt-1 can bind to VEGF and PlGF with high affinity, but it cannot induce proliferation or angiogenesis and is therefore functionally different from the Flt-1 and KDR receptors. sFlt-1 was initially purified from human umbilical endothelial cells and later shown to be produced by trophoblast cells in vivo. As used herein, sFlt-1 includes any sFlt-1 family member or isoform. sFlt-1 can also mean degradation products or fragments that result from enzymatic cleavage of the Flt-1 receptor and that maintain sFlt-1 biological activity. In one example, specific metalloproteinases released from the placenta may cleave the extracellular domain of Flt-1 receptor to release the N-terminal portion of Flt-1 into circulation.

By “specifically binds” is meant a compound or antibody which recognizes and binds a polypeptide of the invention but that does not substantially recognize and bind other molecules in a sample, for example, a biological sample, which naturally includes a polypeptide of the invention. In one example, an antibody that specifically binds soluble endoglin does not bind membrane bound endoglin. In another example, an antibody that specifically binds to soluble endoglin binds to an epitope within the extracellular domain of endoglin, particularly an epitope within amino acids 26 to 437 (excluding the peptide leader sequence), amino acids 40 to 406 of human endoglin (see FIG. 30B), or amino acids 26 to 587 (excluding the peptide leader sequence) that may or may not be unique to soluble endoglin (e.g., in the three dimensional structure of soluble endoglin). In another example, an antibody that specifically binds to soluble endoglin recognizes one or more of the amino acid sequences shown in bold and underlined in FIG. 30B.

By “subject” is meant a mammal, including, but not limited to, a human or non-human mammal, such as a cow, a horse, a sheep, a pig, a goat, a dog, or a cat. Included in this definition are pregnant, post-partum, and non-pregnant mammals.

By “substantially identical” is meant a nucleic acid or amino acid sequence that, when optimally aligned, for example using the methods described below, share at least 60%, 65%, 70%, 75%, 80%, 85%, 90%, 95%, 96%, 97%, 98%, 99%, or 100% sequence identity with a second nucleic acid or amino acid sequence, e.g., an endoglin or soluble endoglin sequence. “Substantial identity” may be used to refer to various types and lengths of sequence, such as full-length sequence, epitopes or immunogenic peptides, functional domains, coding and/or regulatory sequences, exons, introns, promoters, and genomic sequences. Percent identity between two polypeptides or nucleic acid sequences is determined in various ways that are within the skill in the art, for instance, using publicly available computer software such as Smith Waterman Alignment (Smith, T. F. and M. S. Waterman (1981) J Mol Biol 147:195-7); “BestFit” (Smith and Waterman, Advances in Applied Mathematics, 482-489 (1981)) as incorporated into GeneMatcher Plus™, Schwarz and Dayhof (1979) Atlas of Protein Sequence and Structure, Dayhof, M. O., Ed pp 353-358; BLAST program (Basic Local Alignment Search Tool; (Altschul, S. F., W. Gish, et al. (1990) J Mol Biol 215: 403-10), BLAST-2, BLAST-P, BLAST-N, BLAST-X, WU-BLAST-2, ALIGN, ALIGN-2, CLUSTAL, or Megalign (DNASTAR) software. In addition, those skilled in the art can determine appropriate parameters for measuring alignment, including any algorithms needed to achieve maximal alignment over the length of the sequences being compared. In general, for proteins, the length of comparison sequences will be at least 10 amino acids, preferably 20, 30, 40, 50, 60, 70, 80, 90, 100, 110, 120, 130, 140, 150, 200, 250, 300, 350, 400, 437, or at least 587 amino acids or more. For nucleic acids, the length of comparison sequences will generally be at least 25, 50, 100, 125, 150, 200, 250, 300, 350, 400, 450, 500, 550, 600, 650, 700, 800, 900, 1000, 1100, 1200, 1311, or at least 1761 nucleotides or more. It is understood that for the purposes of determining sequence identity when comparing a DNA sequence to an RNA sequence, a thymine nucleotide is equivalent to a uracil nucleotide. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

By “symptoms of pre-eclampsia” is meant any of the following: (1) a systolic blood pressure (BP) >140 mmHg and a diastolic BP>90 mmHg after 20 weeks gestation, (2) new onset proteinuria (1+ by dipstik on urinanaysis, >300 mg of protein in a 24 hour urine collection, or random urine protein/creatinine ratio >0.3), and (3) resolution of hypertension and proteinuria by 12 weeks postpartum. The symptoms of pre-eclampsia can also include renal dysfunction and glomerular endotheliosis or hypertrophy. By “symptoms of eclampsia” is meant the development of any of the following symptoms due to pregnancy or the influence of a recent pregnancy: seizures, coma, thrombocytopenia, liver edema, pulmonary edema, and cerebral edema.

By “transforming growth factor β (TGF-β)” is meant a mammalian growth factor that has TGF-β biological activity and is a member of a family of structurally related paracrine polypeptides found ubiquitously in vertebrates, and prototypic of a large family of metazoan growth, differentiation, and morphogenesis factors (see, for review, Massaque et al. Ann. Rev. Cell. Biol. 6:597-641 (1990); Massaque et al. Trends Cell Biol 4:172-178 (1994); Kingsley Gene Dev. 8:133-146 (1994); and Sporn et al. J. Cell. Biol. 119:1017-1021 (1992). As described in Kingsley, supra, the TGF-β superfamily has at least 25 members, and can be grouped into distinct sub-families with highly related sequences. The most obvious sub-families include the following: the TGF-β sub-family, which comprises at least four genes that are much more similar to TGF-β1 than to other members of the TGF-β superfamily; the bone morphogenetic proteins; the activin sub-family, comprising homo- or hetero-dimers or two sub-units, inhibinβ-A and inhibinβ-B. The decapentaplegic sub-family, which includes the mammalian factors BMP2 and BMP4, which can induce the formation of ectopic bone and cartilage when implanted under the skin or into muscles. The 60A sub-family, which includes a number of mammalian homologs, with osteoinductive activity, including BMP5-8. Other members of the TGF-β superfamily include the gross differentiation factor 1 (GDF-1), GDF-3/VGR-2, dorsalin, nodal, mullerian-inhibiting substance (MIS), and glial-derived neurotrophic growth factor (GDNF). It is noted that the DPP and 60A sub-families are related more closely to one another than to other members of the TGF-β superfamily, and have often been grouped together as part of a larger collection of molecules called DVR (dpp and vgl related). Unless evidenced from the context in which it is used, the term TGF-β as used throughout this specification will be understood to generally refer to members of the TGF-β superfamily as appropriate. (Massague et al., Annu. Rev. Biochem. 67:753-91, 1998; Josso et al., Curr. Op. Gen. Dev., 7:371-377, 1997). TGF-β functions to regulate growth, differentiation, motility, tissue remodeling, neurogenesis, would repair, apoptosis, and angiogenesis in many cell types. TGF-β also inhibits cell proliferation in many cell types and can stimulate the synthesis of matrix proteins.

By “therapeutic amount” is meant an amount that when administered, either by administration directly to the patient or by an ex vivo approach, to a patient suffering from pre-eclampsia or eclampsia is sufficient to cause a qualitative or quantitative reduction in the symptoms of pre-eclampsia or eclampsia as described herein. A “therapeutic amount” can also mean an amount that when administered, either by administration directly to the patient or by an ex vivo approach, to a patient suffering from pre-eclampsia or eclampsia is sufficient to cause a reduction in the expression levels of soluble endoglin or sFlt-1 or an increase in the expression levels of VEGF or PlGF as measured by the assays described herein.

By “treating” is meant administering a compound or a pharmaceutical composition for therapeutic purposes. To “treat disease” or use for “therapeutic treatment” refers to administering treatment to a subject already suffering from a disease to improve the subject's condition. Preferably, the subject is diagnosed as suffering from a pregnancy complication associated with hypertension, such as pre-eclampsia or eclampsia, based on identification of any of the characteristic symptoms described below or the use of the diagnostic methods described herein. To “prevent disease” refers to prophylactic treatment of a subject who is not yet ill, but who is susceptible to, or otherwise at risk of, developing a particular disease. Preferably a subject is determined to be at risk of developing pre-eclampsia or eclampsia using the diagnostic methods described herein. Thus, in the claims and embodiments, treating is the administration to a mammal either for therapeutic or prophylactic purposes.

By “trophoblast” is meant the mesectodermal cell layer covering the blastocyst that erodes the uterine mucosa and through which the embryo receives nourishment from the mother; the cells contribute to the formation of the placenta.

By “vascular endothelial growth factor (VEGF)” is meant a mammalian growth factor that is homologous to the growth factor defined in U.S. Pat. Nos. 5,332,671; 5,240,848; 5,194,596; and Charnock-Jones et al. (Biol. Reproduction, 48: 1120-1128, 1993), and has VEGF biological activity. VEGF exists as a glycosylated homodimer and includes at least four different alternatively spliced isoforms. The biological activity of native VEGF includes the promotion of selective growth of vascular endothelial cells or umbilical vein endothelial cells and induction of angiogenesis. As used herein, VEGF includes any VEGF family member or isoform (e.g., VEGF-A, VEGF-B, VEGF-C, VEGF-D, VEGF-E, VEGF189, VEGF165, or VEGF 121). Preferably, VEGF is the VEGF121 or VEGF165 isoform (Tischer et al., J. Biol. Chem. 266, 11947-11954, 1991; Neufed et al. Cancer Metastasis 15:153-158, 1996), which is described in U.S. Pat. Nos. 6,447,768; 5,219,739; and 5,194,596, hereby incorporated by reference. Also included are mutant forms of VEGF such as the KDR-selective VEGF and Flt-selective VEGF described in Gille et al. (J. Biol. Chem. 276:3222-3230, 2001). As used herein VEGF also includes any modified forms of VEGF such as those described in LeCouter et al. (Science 299:890-893, 2003). Although human VEGF is preferred, the invention is not limited to human forms and can include other animal forms of VEGF (e.g. mouse, rat, dog, or chicken).

By “vector” is meant a DNA molecule, usually derived from a plasmid or bacteriophage, into which fragments of DNA may be inserted or cloned. A recombinant vector will contain one or more unique restriction sites, and may be capable of autonomous replication in a defined host or vehicle organism such that the cloned sequence is reproducible. A vector contains a promoter operably linked to a gene or coding region such that, upon transfection into a recipient cell, an RNA is expressed.

Other features and advantages of the invention will be apparent from the following description of the preferred embodiments thereof, and from the claims.

### BRIEF DESCRIPTION OF THE DRAWINGS

FIG. 1 is a schematic showing the endoglin protein. SP: signal peptide (also referred to as the peptide leader sequence), ZP: zona pellucida domain, CL: potential cleavage site (amino acid 437) for the release of soluble endoglin, TM: transmembrane domain, Cyto: cytoplasmic domain. Once the signal peptide is cleaved, the remaining mature protein starts at the glutamic acid residue at amino acid 26.

FIG. 2A shows the predicted cDNA sequence (SEQ ID NO: 1) of soluble endoglin. FIG. 2B shows the predicted amino acid sequence (SEQ ID NO: 2) of soluble endoglin which includes the signal peptide (amino acids 1-25). It should be noted that the sequence includes the leader peptide sequence that would normally be cleaved in the ER.

FIG. 3 is a Northern blot showing endoglin mRNA levels in placentas from normal pregnancies (N), placentas from preterm pre-eclamptic pregnancies (p) and placentas from term pre-eclamptic pregnancies (P).

FIG. 4 is a western blot showing endoglin protein levels in the placenta. Samples are from two pre-eclamptic patients, p32 and p36, that presented to the Beth Israel Deaconess Medical Center in 2003 and maternal serum from a pregnant woman. The Western blot was probed using a N-terminal antibody obtained from Santa Cruz Biotechnology, Inc., (Santa Cruz, Calif.) that shows both the 110 kD band in the placenta and a smaller 63 kD band that is present in the placenta and the serum samples.

FIG. 5 is a graph that shows the circulating concentrations of soluble endoglin in women with normal pregnancy, mild pre-eclampsia, severe pre-eclampsia and non-pre-eclamptic pregnancies complicated by pre-term delivery. All blood specimens were obtained within 24 hours prior to delivery. Soluble endoglin was measured using an ELISA kit from R & D Systems, MN (Cat # DNDG00). These data show that soluble endoglin levels are significantly elevated in pre-eclamptic patients at the time of clinical disease.

FIG. 6 is a graph showing the mean soluble endoglin concentration for the five different study groups of pregnant women throughout pregnancy during the various gestational age group windows.

FIG. 7 is a graph showing the mean sFlt1 concentrations for the five different study groups of pregnant women throughout pregnancy during the various gestational age group windows.

FIG. 8 is a graph showing the mean PlGF concentrations for the five different study groups of pregnant women throughout pregnancy during the various gestational age group windows.

FIG. 9 is a graph showing the values for the soluble endoglin anti-angiogenic index for pre-eclampsia anti-angiogenesis for samples taken prior to clinical symptoms.

FIG. 10 is a graph showing the mean concentrations of soluble endoglin according to the number of weeks before clinical premature pre-eclampsia (PE<37 weeks).

FIG. 11 is a graph showing the soluble endoglin anti-angiogenic index values according to the number of weeks before clinical premature pre-eclampsia (PE<37 weeks).

FIG. 12 is a graph showing the alteration in soluble endoglin levels throughout pregnancy for term pre-eclampsia (PE>37 weeks) before and after symptoms.

FIG. 13 is a graph showing the alteration in the soluble endoglin anti-angiogenic index levels throughout pregnancy for term pre-eclampsia (PE>37 weeks) before and after symptoms.

FIG. 14 is a graph showing the soluble endoglin levels detected in women during gestational hypertension and before gestational hypertension (1-5 weeks preceding gestational hypertension (during weeks 33-36 of pregnancy)) and normotensive controls.

FIG. 15 is a graph showing the soluble endoglin anti-angiogenic index levels in women during gestational hypertension and before gestational hypertension (1-5 weeks preceding gestational hypertension (during weeks 33-36 of pregnancy)) and normotensive controls.

FIG. 16 is a graph showing the soluble endoglin levels detected during the 33-36 week gestational windows in women with severe SGA, mild SGA, and normotensive controls.

FIG. 17 is a graph showing the soluble endoglin anti-angiogenic index levels detected during the 33-36 week gestational windows in women with severe SGA, mild SGA, and normotensive controls.

FIG. 18 is a graph showing the concentration of sFlt1 and soluble endoglin in the same pregnant patients plotted against each other.

FIG. 19 shows photomicrographs of double immunofluorescence staining of endoglin (red) and smooth muscle actin (green) for pre-eclamptic placentas taken at 25.2 weeks. The antibody used to detect endoglin stains both full-length endoglin and the soluble endoglin. Control placentas for the appropriate gestational windows were derived from patients with pre-term labor.

FIG. 20 shows photomicrographs of double immunofluorescence staining of endoglin (red) and smooth muscle actin (green) for pre-eclamptic placentas taken at 41.3 weeks. The antibody used to detect endoglin stains both full-length endoglin and the soluble endoglin. Control placentas for the appropriate gestational windows were derived from patients with pre-term labor.

FIG. 21A shows an autoradiogram from immunoprecipitation and western blots experiments for endoglin using both pre-eclamptic placentas and serum. FIG. 21B shows an autoradiogram from immunoprecipitation and western blots experiments for endoglin using pre-eclamptic placentas. The three different N and P samples represent individual patients. For both figures commercially available monoclonal antibodies were used for immunoprecipitation and polyclonal antibodies were used for the western blots. Both these antibodies were raised against the N-terminal region of the endoglin protein and detect both the full length and the truncated soluble endoglin protein.

FIG. 22 is a graph showing the results of angiogenesis assays using HUVECs in growth factor reduced matrigels. Angiogenesis assays were performed in the presence of soluble endoglin or sFlt1 or both and the endothelial tube lengths quantitated. C-represents control, E-represents 1 μg/ml of soluble endoglin and S represents 1 μg/ml of sFlt1. E+S represent the combination of 1 μg/ml of E+1 μg/ml of sFlt1. Data represents a mean of three independent experiments.

FIG. 23 is a graph showing the microvascular permeability in several organ beds assessed using Evans blue leakage in mice as described in the materials and methods. C—control (GFP), E—soluble endoglin, S—sFlt1 and S+E—sFlt1+soluble endoglin. Data represents a mean of 4 independent experiments.

FIG. 24 is a graph showing the percent change in rat renal microvessel diameter when subjected to microvascular reactivity experiments in the presence of TGF-β1 (β1) and TGF-β3 (β3) from doses ranging from 200 pg/ml-200 ng/ml. These same experiments were repeated in the presence of soluble endoglin (E) at 1 μg/ml. These data presented are a mean of 4 independent experiments.

FIG. 25 is a graph showing the percent change in the vascular diameter of renal microvessels in the presence of 1 ng/ml of VEGF (V), TGF-β1 (B1) and the combination (V+B1). Also shown is the effect of this combination in the presence of 1 μg/ml each of sFlt1 (S) and soluble endoglin (E) (V+B1+S+E). The data represents a mean of 4 independent experiments.

FIG. 26A is a photograph of a peripheral smear of blood samples taken at the time of sacrifice from pregnant rats injected with the combination of sFlt1 and a control adenoviruses (CMV). FIG. 26B is a photograph of a peripheral smear of blood samples taken at the time of sacrifice from pregnant rats injected with the combination of sFlt and adenoviruses expressing soluble endoglin and demonstrates active hemolysis as evidenced by schistocyes and increased reticulocyte count. Arrowheads represent schistocyte.

FIGS. 27A-D are a series of photomicrographs showing the renal histology (H &E stain) of the various animal groups described in Table 8. FIG. 27A shows the renal histology for the control group with no evidence of glomerular endotheliosis. FIG. 27B shows the renal histology for the soluble endoglin injected group with no evidence of glomerular endotheliosis. FIG. 27C shows the renal histology for sFlt1 injected rats showing moderate endotheliosis (shown by arrow head). FIG. 27D shows the renal histology for the soluble endoglin and sFlt1 injected rats showing extremely swollen glomeruli and severe glomerular endotheliosis with protein resorption droplets in the podocytes. All light micrographs were taken at 60× (original magnification).

FIG. 28 is a graph showing the ELISA results for soluble endoglin (sEng) and sFlt1 in sera of patients with varying degrees of preeclampsia, control pregnancies and four non-pregnant healthy volunteers as described in Example 3. *P<0.05 compared to pre-term controls and ^{#} P<0.05 compared to severe preeclampsia.

FIG. 29 is a graph showing ELISA results for soluble endoglin in a subset of pregnant patients (normal: n=6; preeclampsia: n=11) described in Example 3 with blood drawn pre- (0-12 hours) and post- (48 hours) delivery. * P<0.05 as compared to T=0 samples.

FIG. 30A is a western blot showing soluble endoglin after purification the serum of preeclamptic patients. Fractions 4 and 5 eluted from the 44G4-IgG (anti-Eng) Sepharose were run on SDS-PAGE under reducing conditions and tested by Western blot using a polyclonal antibody to endoglin. The eluted fractions were subjected to mass spectrometry analysis (3 runs). FIG. 30B shows the sequence of human endoglin (SEQ ID NO: 5). Peptides identified by mass spec are shown in bold and underlined. The underlined amino acids represent the transmembrane domain of human cell surface endoglin. Note that the amino acid sequence numbering starts at 26 because amino acids 1-25 represents the leader peptide. Note that the sequence listed as SEQ ID NO: 5 in the sequence listing begins at amino acid 1 so that amino acid 26 in the figure is amino acid 1 in the sequence listing, amino acid 658 in the figure is amino acid 633 in the sequence listing. The numbering of the amino acids is adjusted depending on the reference sequence (i.e., amino acids 26 to 658 for sequences referring to FIG. 30B are the same as amino acids 1 to 633 for sequences referring to SEQ ID NO: 5).

FIG. 31 shows a series of photomicrographs showing soluble endoglin inhibits capillary formation and increases vascular permeability. Angiogenesis assays were performed using HUVEC in growth factor reduced Matrigel™ in the presence of 1 μg of recombinant soluble endoglin, sFlt1, or both, and endothelial tube lengths were quantified. A representative experiment (n=4) is shown with tube lengths in pixels indicated below the panels.

FIG. 32 is a series of graphs showing inhibition of TGF-β1-mediated vascular reactivity in mesenteric vessels by soluble endoglin. Microvascular reactivity of rat mesenteric microvessels was measured in the presence of TGF-β1 or TGF-β3 from 200 pg/ml to 200 ng/ml. The experiments were repeated in the presence of recombinant soluble endoglin at 1 μg/ml. The mean±SE of 4 independent experiments is shown (upper panel). Also shown is the blocking effect of L-NAME on TGFβ1 at 1 ng/ml (lower panel).

FIG. 33 is a series of photomicrographs showing glomerular endotheliosis in pregnant rats. Electron micrographs (EM) of glomeruli from a control pregnant rat (upper panel), soluble endoglin (sEng)-treated pregnant rat (middle panel) and the combination group−soluble endoglin (sEng)+sFlt1 (lower panel) are shown. These photos were taken at 6200× (original magnification) for the upper and middle panel and 5000× (original magnification) for the lower panel.

FIGS. 34 A-H are a series of photomicrographs showing renal, placental and hepatic histological changes and peripheral blood smears in pregnant rats after soluble endoglin and sFlt1 treatment. Placental histology (H &E stain) of control (FIG. 34A), sEng (FIG. 34B), sFlt1 (FIG. 34C) and sFlt1+sEng (FIG. 34D) groups. Both the soluble endoglin and sFlt1 treated animals show diffuse inflammation (arrow heads) at the maternal-fetal junction not seen in controls. There is hemorrhagic infarction and fibrinoid necrosis with lumen obstruction of a maternal vessel (arrow) in the decidua of the sFlt1+sEng treated placenta (FIG. 34D). Scale bar, 200 μM (FIG. 34E-H). Liver histology in the control (FIG. 34E), sEng (FIG. 34F), sFlt1 (FIG. 34G) and sFlt1+sEng (FIG. 34H) groups. Ischemic changes with multifocal necrosis (arrow head) are noted in the sFlt1+sEng group (FIG. 34H). Control group and rats given sEng or sFlt1 showed no changes. Scale bar, 200 μM.

FIGS. 35A-D are a series of graphs and autoradiograms showing recombinant sEng attenuates TGF-β1 binding and activity and its effects on vasodilation via eNOS activation. FIG. 35A is a graph showing the microvascular responses of renal microvessels to 1 ng/ml of VEGF, TGF-β1 and the combination. The effects of 100 ng/ml each of sFlt1 and sEng on the combined response are shown. (n=4). Also shown is the blocking effect of L-NAME on TGFβ1 and VEGF stimulated responses. FIG. 35B is a representative autoradiogram and graph of a dose-dependent increase in [I¹²⁵] TGF-β1 binding to TβRII on mouse endothelial cells. Treatment with 5 nM recombinant soluble endoglin significantly reduced binding at 50 pM and 100 pM (*P<0.05 vs. untreated group). Competition with 40× excess cold TGF-β1 in cells treated with 100 pM [I¹²⁵] TGF-β1 abolished receptor binding and served as background control. FIG. 35C is a graph showing significantly increased TGF-β-induced activation of the Smad 2/3-dependent CAGA-Luc reporter construct transfected in HUVECs and inhibition by treatment with sEng. (n=3, **P<0.01 vs. sEng untreated group). FIG. 35D is a representative western blots and graph (n=4) showing significant dephosphorylation at eNOS Thr495 following treatment with TGF-β3 and attenuation by sEng (*P<0.05 vs. untreated). Phosphorylation was unchanged at Ser1177 and total levels of eNOS remained constant throughout the experiments.

FIG. 36 shows two western blots of rat plasma demonstrating expression of the recombinant sFlt1 and soluble endoglin. Upper panel: Plasma specimens from pregnant rats (at early third trimester) were used as described in Methods. Lanes 1, 2 and 3 represent 200 pg, 500 pg and 2 ng of recombinant mouse Flt1-Fe protein used as a positive control. 20 μl of plasma specimens from one control rat and two sFlt1 treated rats are shown. sFlt1 (53 kDa) band was detected in the sFlt1 treated rats. Quantitation of the sFlt1 expression was performed using commercially available ELISA (Table 8). Lower panel: Plasma specimens from pregnant rats were used (at early third trimester) to detect sEng expression. Lane 1 represents 500 pg of recombinant human soluble endoglin and lanes 2 and 3 represent 30 μl of plasma from sEng treated and control rats respectively. The blot shows no soluble endoglin in control rats but robust expression of recombinant sEng in treated rats. Quantitation of soluble endoglin was performed using a commercially available ELISA (Table 8).

FIG. 37 is a graph showing the distribution of delta sFlt1 and delta sEng (first trimester-second trimester values) in controls, all pre-eclampsia and in pre-eclampsia <37 weeks.

FIG. 38 is a graph showing the distribution of sFlt×sEng product in the first trimester (product 1), in the second trimester (product 2), delta product (product 1-product 2) in controls, all pre-eclampsia and in pre-eclampsia <37 weeks.

FIG. 39 is a graph showing the risk of pre-eclampsia according to tertiles of delta product. The increase in risk of preterm preeclampsia in the group whose delta product levels were greater than +1 [aOR 5.5, 95% CI 1.4-22.4], compared to women whose delta product was less than −1 was statistically significant (P<0.05).

FIG. 40A is a western blot showing endoglin is necessary for TGF-β1 induced dephosphorylation of eNOS at Thr495. FIG. 40B is a graph showing the percent of eNOS Thr495 phosphorylation relative to total eNOS. The results show that the level of phosphorylated Thr495 decreases in the presence of TGF-β1 in the presence of soluble endoglin but not in the absence of soluble endoglin.

### DETAILED DESCRIPTION

We have discovered that soluble endoglin levels are elevated in blood serum samples taken from women with a pregnancy related hypertensive disorder, such as pre-eclampsia or eclampsia. Soluble endoglin may be formed by cleavage of the extracellular portion of the membrane bound form by proteolytic enzymes. The lack of detection of alternate splice variants in placenta and the partial peptide sequence of purified soluble endoglin as described herein suggest that it is an N-terminal cleavage product of full-length endoglin. Excess soluble endoglin may be depleting the placenta of necessary amounts of these essential angiogenic and mitogenic factors. We have discovered that excess circulating concentrations of soluble endoglin and sFlt1 in patients with preeclampsia contribute to the pathogenesis of pre-eclampsia and other pregnancy related hypertensive disorders. We have also discovered that soluble endoglin interferes with TGF-β1 and TGF-β3 binding to its receptor leading to decreased signaling such as a reduction in eNOS activation in endothelial cells, thereby disrupting key homeostatic mechanisms necessary for maintenance of vascular health. These data suggest a crucial role for endoglin in linking TGF-β receptor activation to NO synthesis. In addition, we have discovered that soluble endoglin and sFlt1 act in concert to induce vascular damage and pregnancy related hypertensive disorders, such as pre-eclampsia or eclampsia, by interfering with TGF-β1 and VEGF signaling respectively, likely via inhibition of the downstream activation of eNOS.

The present invention features the use of therapeutic agents that interfere with soluble endoglin binding to growth factors, agents that reduce soluble endoglin expression or biological activity, or agents that increase levels of growth factors, can be used to treat or prevent pregnancy related hypertensive disorders, such as pre-eclampsia or eclampsia in a subject. Such agents include, but are not limited to, antibodies that bind to soluble endoglin and inhibit soluble endoglin biological activity, oligonucleotides for antisense or RNAi that reduce levels of soluble endoglin, compounds that increase the levels of growth factors that bind to soluble endoglin, compounds that prevent the proteolytic cleavage of the membrane bound form of endoglin thereby preventing the release of soluble endoglin, and small molecules that bind soluble endoglin and block the growth factor binding site. Additionally or alternatively, the invention features the use of any compound (e.g., polypeptide, small molecule, antibody, nucleic acid, and mimetic) that increases the level or biological activity of TGF-β, eNOS, and PGI₂ to treat or prevent pregnancy related hypertensive disorders, such as pre-eclampsia or eclampsia in a subject. Additionally, the invention features the use of any compound that decreases the level of sFlt-1 or increases the level or activity of VEGF or PlGF (see for example, U.S. Patent Application Publication Numbers 20040126828, 20050025762, and 20050170444 and PCT Publication Numbers WO 2004/008946 and WO 2005/077007) in combination with any of the therapeutic compounds described above to treat or prevent pregnancy related hypertensive disorders, such as pre-eclampsia or eclampsia, in a subject. In addition, the invention features the use of soluble endoglin, eNOS, TGF-β, of PGI₂, either alone or in combination, as a diagnostic marker of pregnancy related hypertensive disorders, including pre-eclampsia and eclampsia.

While the detailed description presented herein refers specifically to soluble endoglin, TGF-β1, eNOS, sFlt-1, VEGF, or PlGF, it will be clear to one skilled in the art that the detailed description can also apply to family members, isoforms, and/or variants of soluble endoglin, TGF-β, eNOS, sFlt-1, VEGF, or PlGF.

### Diagnostics

We have discovered that soluble endoglin levels are elevated in blood serum samples taken from women with a pregnancy related hypertensive disorder, such as pre-eclampsia or eclampsia. Soluble endoglin starts rising 6-10 weeks before clinical symptoms of preeclampsia. Accordingly, a diagnostic test measuring soluble endoglin and sFlt1, optionally in combination with free PlGF, in the serum will have enhanced sensitivity and specificity, and provide a powerful tool in the prevention of preeclampsia-induced mortality. The diagnostic test can also include measuring the levels of free VEGF; TGF-β family members, preferably TGF-β1, TGF-β3, free activin-A, BMP2, BMP7; NOS, preferably eNOS; or PGI2, either alone or in any combination thereof. An alteration in the levels of any of these proteins is diagnostic of a pregnancy related hypertensive disorder, such as pre-eclampsia or eclampsia. In one example, a decrease in the levels of free BMP2, BMP7, or activin A is diagnostic of a pregnancy related hypertensive disorder, such as pre-eclampsia or eclampsia.

While the methods described herein refer to pre-eclampsia and eclampsia specifically, it should be understood that the diagnostic and monitoring methods of the invention apply to any pregnancy related hypertensive disorder including, but not limited to, gestational hypertension, pregnancy with a small for gestational age (SGA) infant, HELLP, chronic hypertension, pre-eclampsia (mild, moderate, and severe), and eclampsia.

Levels of soluble endoglin, either free, bound, or total levels, are measured in a subject sample and used as an indicator of pre-eclampsia, eclampsia, or the propensity to develop such conditions.

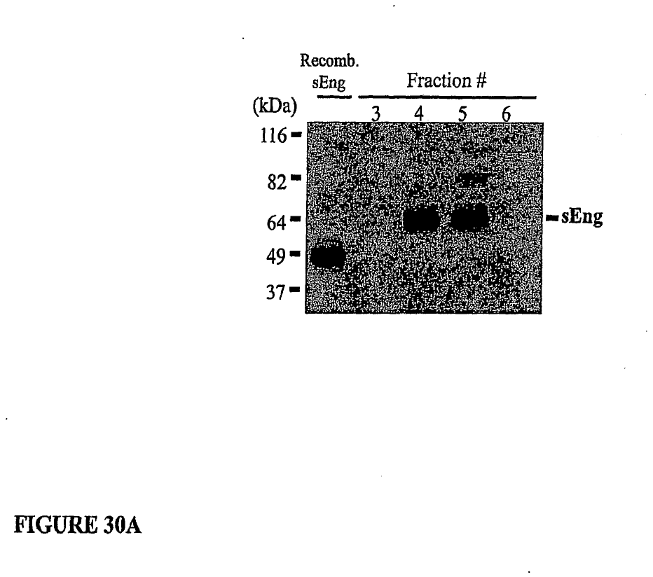

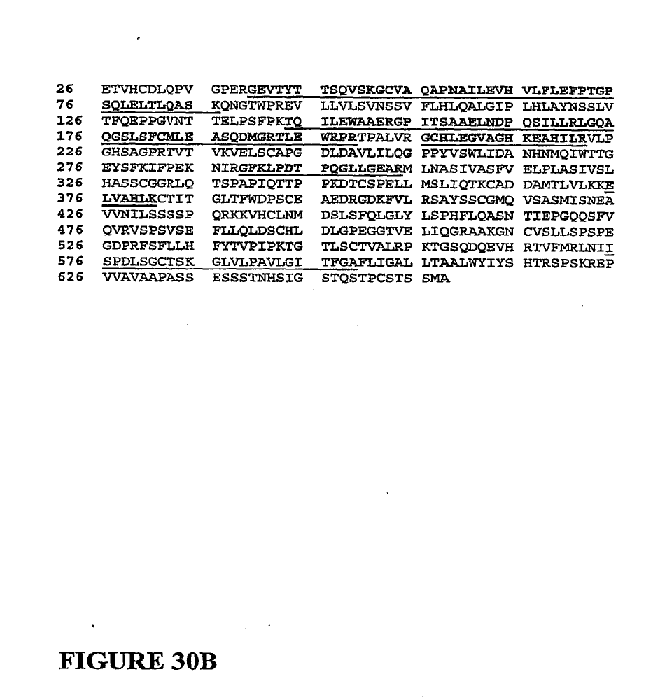

A subject having pre-eclampsia, eclampsia, or a predisposition to such conditions will show an increase in the expression of a soluble endoglin polypeptide. The soluble endoglin polypeptide can include full-length, soluble endoglin, degradation products, alternatively spliced isoforms of soluble endoglin, enzymatic cleavage products of soluble endoglin, and the like. An antibody that specifically binds a soluble endoglin polypeptide may be used for the diagnosis of pre-eclampsia or eclampsia or to identify a subject at risk of developing such conditions. One example of an antibody useful in the methods of the invention is a monoclonal antibody against the N-terminal region of endoglin that is commercially available from Santa Cruz Biotechnology, Inc. (cat #sc-20072). Additional examples include antibodies that specifically bind the extracellular domain of endoglin (e.g., amino acids 1 to 437 of endoglin, amino acids 1 to 587 of endoglin, or any of the amino acid sequences shown in bold and underlined in FIG. 30B). A variety of protocols for measuring an alteration in the expression of such polypeptides are known, including immunological methods (such as ELISAs and RIAs), and provide a basis for diagnosing pre-eclampsia or eclampsia or a risk of developing such conditions.

Increased levels of soluble endoglin are a positive indicator of pre-eclampsia or eclampsia. For example, if the level of soluble endoglin is increased relative to a normal reference (e.g., 10%, 20%, 30%, 40%, 50%, 60%, 70%, 80%, 90% or more), or increases over time in one or more samples from a subject, this is considered a positive indicator of pre-eclampsia or eclampsia. Additionally, any detectable alteration in levels of soluble endoglin, sFlt-1, VEGF, or PlGF relative to normal levels is indicative of eclampsia, pre-eclampsia, or the propensity to develop such conditions. Normally, circulating serum concentrations of soluble endoglin range from 2-7 ng/ml during the non-pregnant state and from 10-20 ng/ml during normal pregnancy. Elevated serum levels, greater than 15 ng/ml, preferably greater than 20 ng/ml, and most preferably greater than 25 ng/ml or more, of soluble endoglin is considered a positive indicator of pre-eclampsia or eclampsia.

In one embodiment, the level of soluble endoglin is measured in combination with the level of sFlt-1, VEGF, or PlGF polypeptide or nucleic acid, or any combination thereof. Methods for the measurement of sFlt-1, VEGF, and PlGF are described in U.S. Patent Application Publication Numbers 20040126828, 20050025762, and 20050170444 and PCT Publication Numbers WO 2004/008946 and WO 2005/077007, hereby incorporated by reference in their entirety. In additional preferred embodiments, the body mass index (BMI) and gestational age of the fetus is also measured and included the diagnostic metric.

In another embodiment, the level of TGF-β1, TGF-β3, or eNOS polypeptide or nucleic acid is measured in combination with the level of soluble endoglin, sFlt-1, VEGF, or PlGF polypeptide or nucleic acid. Antibodies useful for the measurement of TGF-β1 and β3 polypeptide levels are commercially available, for example, from Abcam, Abgent, BD Biosciences Pharmingen, Chemicon, GeneTex, and R&D Systems. The level of PGI₂ can also be used in combination with the level of any of the above polypeptides. PGI₂ levels can be determined, for example, using the PGI₂ receptor as a binding molecule in any of the diagnostic assays described above, or using, for example, the urinary prostacyclin colorimetric ELISA kit (Assay Designs). Antibodies useful for the measurement of eNOS polypeptide levels are commercially available, for example, from Research Diagnostics Inc., Santa Cruz, Cayman Chemicals, and BD Biosciences.

In another embodiment, the biological activity of any one or more of TGF-β1, TGF-β3, or eNOS polypeptide is measured in combination with the biological activity of soluble endoglin, sFlt-1, VEGF, or PlGF polypeptide and a decrease in the biological activity is a positive indicator of pre-eclampsia or eclampsia. The biological activity can be measured, for example using an assay for enzymatic activity or for the downstream signaling activity. In one example, the enzymatic activity of eNOS is determined by measuring citrulline conversion and a decrease in the enzymatic activity of eNOS is a positive indicator of pre-eclampsia or eclampsia.

In one embodiment, a metric incorporating soluble endoglin, sFlt-1, VEGF, or PlGF, or any combination therein, is used to determine whether a relationship between levels of at least two of the proteins is indicative of pre-eclampsia or eclampsia. In one example, the metric is a PAAI (sFlt-1/VEGF+PlGF), which is used, in combination with soluble endoglin measurement, as an anti-angiogenic index that is diagnostic of pre-eclampsia, eclampsia, or the propensity to develop such conditions. If the level of soluble endoglin is increased relative to a reference sample (e.g., 1.5-fold, 2-fold, 3-fold, 4-fold, or even by as much as 10-fold or more), and the PAAI is greater than 10, more preferably greater than 20, then the subject is considered to have pre-eclampsia, eclampsia, or to be in imminent risk of developing the same. The PAAI (sFlt-1/VEGF+PlGF) ratio is merely one example of a useful metric that may be used as a diagnostic indicator. It is not intended to limit the invention. Virtually any metric that detects an alteration in the level of soluble endoglin, sFlt-1, PlGF, or VEGF, or any combination thereof, in a subject relative to a normal control may be used as a diagnostic indicator. Another example is the following soluble endoglin anti-angiogenic index: (sFlt-1+0.25(soluble endoglin polypeptide))/PlGF. An increase in the value of the soluble endoglin metric over time or compared to a reference sample or value is a diagnostic indicator of pre-eclampsia or eclampsia. A soluble endoglin index above 100, preferably above 200 is a diagnostic indicator of pre-eclampsia or eclampsia. Additional examples include the following indexes: (soluble endoglin+sFlt-1)/PlGF or sFlt-1×soluble endoglin.

Another example includes the measurement of the levels of sFlt-1 and soluble endoglin in the first and second trimesters in a subject and calculating the delta value of sFlt1×soluble endoglin (sEng) in each trimester using the following equation: [dproduct=(sFlt1×sEng) in the second trimester−(sFlt1×sEng) in the first trimester], where a value greater than 0, 1, 2, or more, including fractions thereof (e.g., a positive value) is a diagnostic indicator of pre-eclampsia or eclampsia. A positive value can also be an indicator of pre-term pre-eclampsia. Such a measurement can be taken on numerous occasions during the first and second trimesters and the dproduct can be followed over time. In addition, the dproduct of the sFlt-1 level (dsFlt-1) and the sEng level (dsEng) alone can also be calculated between the first and second trimesters, where a value greater than 0, 1, 2, or more, including fractions thereof (e.g., a positive value) for (dsFlt-1) or (dsEng) is a diagnostic indicator of pre-eclampsia or eclampsia.

In addition, the metric can further include the level of TGF-β1, TGF-β3, PGI₂, or eNOS polypeptide. Any of the metrics can further include the BMI of the mother or the GA of the infant.

Standard methods may be used to measure levels of soluble endoglin, free VEGF, free PlGF, sFlt-1, TGF-β1, TGF-β3, PGI₂, or eNOS polypeptide in any bodily fluid, including, but not limited to, urine, serum, plasma, saliva, amniotic fluid, or cerebrospinal fluid. Such methods include immunoassay, ELISA, western blotting using antibodies directed to soluble endoglin, free VEGF, free PlGF, sFlt-1, TGF-β1, TGF-β3, PGI₂, or eNOS polypeptide and quantitative enzyme immunoassay techniques such as those described in Ong et al. (Obstet. Gynecol. 98:608-611, 2001) and Su et al. (Obstet. Gynecol., 97:898-904, 2001). ELISA is the preferred method for measuring levels of soluble endoglin, VEGF, PlGF, sFlt-1, TGF-β1, TGF-β3, PGI₂, or eNOS polypeptide. Preferably, soluble endoglin is measured alone or in combination with any one or more of the remaining polypeptides.

Oligonucleotides or longer fragments derived from an endoglin, sFlt-1, PlGF, or VEGF nucleic acid sequence may be used as a probe not only to monitor expression, but also to identify subjects having a genetic variation, mutation, or polymorphism in an endoglin, sFlt-1, PlGF, or VEGF nucleic acid molecule that are indicative of a predisposition to develop the pre-eclampsia or eclampsia. Such methods are described in detail in Abdalla et al., Hum. Mutat. 25:320-321 (2005), U.S. Patent Application Publication No. 2006/0067937 and PCT Publication No. WO 06/034507. Preferred oligonucleotides will hybridize at high stringency to the extracellular domain of endoglin or to any nucleic acid sequence encoding any of the peptides shown in bold and underlined in FIG. 30B.

The measurement of any of the nucleic acids or polypeptides described herein can occur on at least two different occasions and an alteration in the levels as compared to normal reference levels over time is used as an indicator of pre-eclampsia, eclampsia, or the propensity to develop such conditions.

In one example, the level of a soluble endoglin polypeptide or nucleic acid present in the bodily fluids of a subject having pre-eclampsia, eclampsia, or the propensity to develop such conditions may be increased by as little as 10%, 20%, 30%, or 40%, or by as much as 50%, 60%, 70%, 80%, 90%, 95%, 96%, 97%, 98%, 99% or more relative to levels in a normal control subject or relative to a previous sampling obtained from the same bodily fluids of the same subject. In another example, the level of a soluble endoglin polypeptide or nucleic acid in the bodily fluids of a subject having pre-eclampsia, eclampsia, or the propensity to develop such conditions may be altered by as little as 10%, 20%, 30%, or 40%, or by as much as 50%, 60%, 70%, 80%, 90%, 95%, 96%, 97%, 98%, 99% over time from one measurement to the next.

The level of sFlt-1, VEGF, or PlGF measured in combination with the level of soluble endoglin in the bodily fluids of a subject having pre-eclampsia, eclampsia, or the propensity to develop such conditions may be altered by as little as 10%, 20%, 30%, or 40%, or by as much as 50%, 60%, 70%, 80%, 90%, 95%, 96%, 97%, 98%, 99% or more relative to the level of sFlt-1, VEGF, or PlGF in a normal control. The level of sFlt-1, VEGF, or PlGF measured in combination with the level of soluble endoglin in the bodily fluids of a subject having pre-eclampsia, eclampsia, or the propensity to develop such conditions may be altered by as little as 10%, 20%, 30%, or 40%, or by as much as 50%, 60%, 70%, 80%, 90%, 95%, 96%, 97%, 98%, 99% over time from one measurement to the next.

In one embodiment, a subject sample of a bodily fluid (e.g., urine, plasma, serum, amniotic fluid, or cerebrospinal fluid) is collected early in pregnancy prior to the onset of pre-eclampsia symptoms. In another example, the sample can be a tissue or cell collected early in pregnancy prior to the onset of pre-eclampsia symptoms. Non-limiting examples of tissues and cells include placental tissue, placental cells, circulating endothelial cells, and leukocytes such as monocytes. In humans, for example, maternal blood serum samples are collected from the antecubital vein of pregnant women during the first, second, or third trimesters of the pregnancy. Preferably, the assay is carried out during the first trimester, for example, at 4, 6, 8, 10, or 12 weeks, or any interval therein, or during the second trimester, for example at 14, 16, 18, 20, 22, or 24 weeks, or any interval therein. In one example, the assay is carried out between 13 and 16 weeks of pregnancy. Such assays may also be conducted at the end of the second trimester or the third trimester, for example at 26, 28, 30, 32, 34, 36, or 38 weeks, or any interval therein. It is preferable that levels of soluble endoglin and/or any of the additional polypeptides described herein be measured twice during this period of time. For the diagnosis of post-partum pre-eclampsia or eclampsia, assays for soluble endoglin may be carried out postpartum. For the diagnosis of a predisposition to pre-eclampsia or eclampsia, the assay is carried out prior to the onset of pregnancy or prior to the development of symptoms of pre-eclampsia or eclampsia. In one example, for the monitoring and management of therapy, the assay is carried out during the pregnancy after the diagnosis of pre-eclampsia, and/or during therapy.

In one particular example, serial blood samples can be collected during pregnancy and the levels of soluble endoglin polypeptide and/or any of the additional polypeptides of the invention determined by ELISA. In another example, a sample is collected during the second trimester and early in the third trimester and in increase in the level of soluble endoglin of any of the other polypeptides of the invention from the first sampling to the next is indicative of pre-eclampsia or eclampsia, or the propensity to develop either.

The invention also include the measurement of any soluble endoglin binding protein (e.g., TGF-β1, TGF-β3, activin-A, BMP-2, and BMP-7) or downstream mediators of soluble endoglin signaling (e.g., PGI₂ and eNOS) in a bodily fluid from a subject, preferably urine, and an alteration (e.g., increase or decrease) in the level of the soluble endoglin binding protein is indicative of pre-eclampsia or eclampsia. The methods and timing for measurement of soluble endoglin described herein can also be used for the measurement of any of the soluble endoglin binding protein, PGI₂ or eNOS.

In veterinary practice, assays may be carried out at any time during the pregnancy, but are, preferably, carried out early in pregnancy, prior to the onset of pre-eclampsia symptoms. Given that the term of pregnancies varies widely between species, the timing of the assay will be determined by a veterinarian, but will generally correspond to the timing of assays during a human pregnancy.

The diagnostic methods described herein can be used individually or in combination with any other diagnostic method described herein or known in the art for a more accurate diagnosis of the presence of, severity of, or estimated time of onset of pre-eclampsia or eclampsia. In addition, the diagnostic methods described herein can be used in combination with any other diagnostic methods determined to be useful for the accurate diagnosis of the presence of, severity of, or estimated time of onset of pre-eclampsia or eclampsia.

The diagnostic methods described herein can also be used to monitor and manage pre-eclampsia or eclampsia in a subject. In one example, a therapy is administered until the blood, plasma, or serum soluble endoglin level is less than 25 ng/ml or until the serum soluble endoglin levels (or soluble endoglin binding protein, PGI₂, or eNOS level) return to the baseline level-determined before onset of pre-eclampsia or eclampsia. In another example, if a subject is determined to have an increased level of soluble endoglin relative to a normal control then the therapy can be administered until the serum PlGF level rises to approximately 400 pg/mL or a return to baseline level prior to onset of pre-eclampsia or eclampsia. In this embodiment, the levels of soluble endoglin, sFlt-1, PlGF, VEGF, soluble endoglin binding protein, PGI₂, eNOS or any and all of these, are measured repeatedly as a method of not only diagnosing disease but monitoring the treatment and management of the pre-eclampsia and eclampsia.

### Diagnostic Kits

The invention also provides for a diagnostic test kit. For example, a diagnostic test kit can include binding agents (e.g., polypeptides or antibodies) that specifically bind to soluble endoglin and means for detecting, and more preferably evaluating, binding between the binding agent and the soluble endoglin polypeptide. For detection, either the binding agent or the soluble endoglin polypeptide is labeled, and either the binding agent or the soluble endoglin polypeptide is substrate-bound, such that soluble endoglin polypeptide-binding agent interaction can be established by determining the amount of label attached to the substrate following binding between the binding agent and the soluble endoglin polypeptide. A conventional ELISA is a common, art-known method for detecting antibody-substrate interaction and can be provided with the kit of the invention. Soluble endoglin polypeptides can be detected in virtually any bodily fluid including, but not limited to urine, serum, plasma, saliva, amniotic fluid, or cerebrospinal fluid. The invention also provides for a diagnostic test kit that includes a soluble endoglin nucleic acid that can be used to detect and determine levels of soluble endoglin nucleic acids. A kit that determines an alteration, for example, an increase, in the level of soluble endoglin polypeptide relative to a reference, such as the level present in a normal control, is useful as a diagnostic kit in the methods of the invention.

The diagnostic kits of the invention can include antibodies or nucleic acids for the detection of sFlt-1, VEGF, or PlGF polypeptides or nucleic acids as described U.S. Patent Application Publication Numbers 20040126828, 20050025762, and 20050170444 and PCT Publication Numbers WO 2004/008946 and WO 2005/077007.

In another embodiment, the kit can also include binding agents for the detection of soluble endoglin ligands including but not limited to TGF-β1, TGF-β3, or PGI₂ or eNOS polypeptides. Antibodies useful for the measurement of TGF-β1 and β3 polypeptide levels are commercially available, for example, from Abcam, Abgent, BD Biosciences Pharmingen, Chemicon, GeneTex, and R&D Systems. Antibodies useful for the measurement of eNOS polypeptide levels are commercially available, for example, from Research Diagnostics Inc., Santa Cruz, Cayman Chemicals, and BD Biosciences. Binding agents for the detection of PGI₂ levels can also be included and include for example the PGI₂ receptor, or fragments thereof, as a binding molecule in any of the diagnostic assays described above, or using, for example, the urinary prostacyclin colorimetric ELISA kit (Assay Designs). A kit that determines an alteration, for example, a decrease, in the level of eNOS, TGF-β1 or β3 polypeptide or PGI₂ relative to a normal reference or standard or level, such as the level present in a normal control, is useful as a diagnostic kit in the methods of the invention. A kit that determines an alteration, for example, a decrease in the level of soluble endoglin or an increase in the level of a soluble endoglin binding protein (e.g., TGF-β1, TGF-β3, activin A, BMP2, and BMP 7) or downstream mediators of soluble endoglin signaling (e.g., eNOS and PGI₂) relative to a positive reference standard or level is useful for monitoring the treatment of pre-eclampsia or eclampsia.

Desirably, the kit includes any of the components needed to perform any of the diagnostic methods described above. For example, the kit desirably includes a membrane, where the soluble endoglin binding agent or the agent that binds the soluble endoglin binding agent is immobilized on the membrane. The membrane can be supported on a dipstick structure where the sample is deposited on the membrane by placing the dipstick structure into the sample or the membrane can be supported in a lateral flow cassette where the sample is deposited on the membrane through an opening in the cassette.

The diagnostic kits also generally include a label or instructions for the intended use of the kit components and a reference sample or purified proteins to be used to establish a standard curve. In one example, the kit contains instructions for the use of the kit for the diagnosis of a pregnancy related hypertensive disorder, such as pre-eclampsia, eclampsia, or the propensity to develop pre-eclampsia or eclampsia. In yet another example, the kit contains instructions for the use of the kit to monitor therapeutic treatment or dosage regimens for the treatment of pre-eclampsia or eclampsia. The diagnostic kit may also include a label or instructions for the use of the kit to determine the PAAI or soluble endoglin anti-angiogenesis index of the subject sample and to compare the PAAI or soluble endoglin anti-angiogenesis index to a reference sample value. It will be understood that the reference sample values will depend on the intended use of the kit. For example, the sample can be compared to a normal reference value, wherein an increase in the PAAI or soluble endoglin anti-angiogenesis index or in the soluble endoglin value is indicative of pre-eclampsia or eclampsia, or a predisposition to pre-eclampsia or eclampsia. In another example, a kit used for therapeutic monitoring can have a reference PAAI or soluble endoglin anti-angiogenesis index value or soluble endoglin value that is indicative of pre-eclampsia or eclampsia, wherein a decrease in the PAAI or soluble endoglin anti-angiogenesis index value or a decrease in the soluble endoglin value of the subject sample relative to the reference sample can be used to indicate therapeutic efficacy or effective dosages of therapeutic compounds. A standard curve of levels of purified protein within the normal or positive reference range, depending on the use of the kit, can also be included.

### Therapeutics

The present invention features methods and compositions for treating or preventing pre-eclampsia or eclampsia in a subject. Given that levels of soluble endoglin are increased in subjects having pre-eclampsia, eclampsia, or having a predisposition to such conditions, any compound that decreases the expression levels and/or biological activity of a soluble endoglin polypeptide or nucleic acid molecule is useful in the methods of the invention. Such compounds include TGF-β1, TGF-β3, activin-A, BMP2, or BMP7, that can disrupt soluble endoglin binding to ligands; a purified antibody or antigen-binding fragment that specifically binds soluble endoglin; antisense nucleobase oligomers; and dsRNAs used to mediate RNA interference. Additional useful compounds include any compounds that can alter the biological activity of soluble endoglin, for example, as measured by an angiogenesis assay. Exemplary compounds and methods are described in detail below. These methods can also be combined with methods to decrease sFlt-1 levels or to increase VEGF or PlGF levels or decrease sFlt-1 levels as described in PCT Publication Number WO 2004/008946 and U.S. Patent Publication Nos. 20040126828 and 20050170444. In addition, any compound that increases the level or biological activity of TGF-β1 or 3, eNOS, or PGI2 are useful in the methods of the invention. Exemplary compounds and methods are described in detail below.

It should be noted that we have discovered that the soluble endoglin and sFlt-1 pathways may be functioning in a cooperative manner to further the pathogenesis of pre-eclampsia or eclampsia. Therefore, the invention includes any combination of any of the methods or compositions described herein for the treatment or prevention of a pregnancy related hypertensive disorder. For example, a compound that targets the soluble endoglin pathway (e.g., downregulates soluble endoglin expression or biological activity or upregulates TGF-β, eNOS, or PGI₂ expression or biological activity) can be used in combination with a compound that targets the sFlt-1 pathway (e.g., downregulates sFlt-1 expression or biological activity or upregulates VEGF or PlGF expression of biological activity) for the treatment or prevention of a pregnancy related hypertensive disorder.

### Therapeutics Targeting the TGF-β3 Signaling Pathway

TGF-β is the prototype of a family of at least 25 growth factors which regulate growth, differentiation, motility, tissue remodeling, neurogenesis, wound repair, apoptosis, and angiogenesis in many cell types. TGF-β also inhibits cell proliferation in many cell types and can stimulate the synthesis of matrix proteins. Unless evidenced from the context in which it is used, the term TGF-β as used throughout this specification will be understood to generally refer to any and all members of the TGF-β superfamily as appropriate. Soluble endoglin binds several specific members of the TGF-β family including TGF-β1, TGF-β3, activin, BMP-2 and BMP-7, and may serve to deplete the developing fetus or placenta of these necessary mitogenic and angiogenic factor. The present invention features methods of increasing the levels of these ligands to bind to soluble endoglin and to neutralize the effects of soluble endoglin.

### Soluble Endoglin Ligands as Therapeutic Compounds

In a preferred embodiment of the present invention, purified forms of any soluble endoglin ligand such as TGF-β family proteins, including but not limited to TGF-β1, TGF-β3, activin-A, BMP2, and BMP7, are administered to the subject in order to treat or prevent pre-eclampsia or eclampsia.

Purified TGF-β family proteins include any protein with an amino acid sequence that is homologous, more desirably, substantially identical to the amino acid sequence of TGF-β1 or TGF-β3, or any known TGF-β family member, that can induce angiogenesis. Non-limiting examples include human TGF-β1 (Cat #240-B-002) and human TGF-β3 (Cat #243-B3-002) from R & D Systems, MN. Preferred TGF-β family proteins useful in the methods of the invention will have the ability to bind to soluble endoglin (e.g., Barbara et al, J. Biol. Chem. 274:584-94 (1999)).

Therapeutic Compounds that Inhibit Proteolytic Cleavage of Endoglin

We have identified a potential cleavage site in the extracellular domain of endoglin where a proteolytic enzyme could cleave the membrane bound form of endoglin, releasing the extracellular domain as a soluble form. Our sequence alignments of the cleavage site suggest that a matrix metalloproteinase (MMP) may be responsible for the cleavage and release of soluble endoglin. Alternatively, a cathepsin or an elastase may also be involved in the cleavage event. MMPs are also known as collagenases, gelatinases, and stromelysins and there are currently 26 family members known (for a review see Whittaker and Ayscough, Cell Transmissions 17:1 (2001)). A preferred M is MMP9, which is known to be up-regulated in placentas from pre-eclamptic patients (Lim et al., Am. J. Pathol. 151:1809-1818, 1997). The activity of MMPs is controlled through activation of pro-enzymes and inhibition by endogenous inhibitors such as the tissue inhibitors of metalloproteinases (TIMPS). Inhibitors of MMPs are zinc binding proteins. There are 4 known endogenous inhibitors (TIMP 1-4), which are reviewed in Whittaker et al., supra. One preferred MMP inhibitor is the inhibitor of membrane type-MMP1 that has been shown to cleave betaglycan, a molecule that shares similarity to endoglin (Velasco-Loyden et al., J. Biol. Chem. 279:7721-7733 (2004)). In addition, a variety of naturally-occurring and synthetic MMP inhibitors have been identified and are also reviewed in Whittaker et al., supra. Examples include antibodies directed to MMPs, and various compounds including marimastat, batimastat, CT1746, BAY 12-9566, Prinomastat, CGS-27023A, D9120, BMS275291 (Bristol Myers Squibb), and trocade, some of which are currently in clinical trials. Given the potential role of MMPs, cathepsins, or elastases in the release and up-regulation of soluble endoglin levels, the present invention also provides for the use of any compound, such as those described above, known to inhibit the activity of any MMP, cathepsin, or elastase involved in the cleavage and release of soluble endoglin, for the treatment or prevention of pre-eclampsia or eclampsia in a subject.

Therapeutic Compounds that Increase Soluble Endoglin Binding Proteins

The present invention provides for the use of any compound known to stimulate or increase blood serum levels of soluble endoglin binding proteins, including but not limited to TGF-β1, TGF-β3, activin-A, BMP2, and BMP7, for the treatment or prevention of pre-eclampsia in a subject. These compounds can be used alone or in combination with the purified proteins described above or any of the other methods used to increase TGF-β family proteins protein levels described herein. In one example, cyclosporine is used at a dosage of 100-200 mg twice a day to stimulate TGF-β1 production.

Therapeutic Compounds that Alter the Anti-Angiogenic Activity of Soluble Endoglin

Additional therapeutic compounds can be identified using angiogenesis assays. For example, pre-eclamptic serum having elevated levels of soluble endoglin are added to a matrigel tube formation assay will induce an anti-angiogenic state. Test compounds can then be added to the assay and a reversion in the anti-angiogenic state by 10%, 20%, 30%, 40%, 50%, 60%, 70%, 80%, 90% or more indicates that the compound can reduce the biological activity of soluble endoglin and is useful as a therapeutic compound.

Therapeutic Compounds that Increase the Levels or Biological Activity of NOS

NOS is a complex enzyme containing several cofactors, a heme group which is part of the catalytic site, an N-terminal oxygenase domain, which belongs to the class of haem-thiolate proteins, and a C-terminal reductase domain which is homologous to NADPH:P450 reductase. NOS produces NO by catalysing a five-electron oxidation of a guanidino nitrogen of L-arginine (L-Arg).

eNOS activation involves a coordinated increase in Ser1177 phosphorylation and Thr495 dephosphorylation. We have discovered that TGF-β1 dephosphorylates eNOS at Thr495, which is necessary to increase the Ca2+ sensitivity and enzyme activity and may work synergistically with VEGF, which activates eNOS by phosphorylating Ser1177.

Accordingly, any compound (e.g., polypeptide, nucleic acid molecule, small molecule compound, or antibody) that increases the level (e.g., by increasing stability, transcription or translation, or decreasing protein degradation) or biological activity of NOS, particularly eNOS, or any compound that prevents the downregulation of eNOS activity is useful in the methods of the invention. Such compounds include purified NOS, preferably eNOS, or biologically active fragments thereof, nucleic acids encoding NOS, preferably eNOS, or biologically active fragments thereof, statins, vanadate, hepatocyte growth factor, phosphoinositide 3-kinase (PI3K), Akt, VEGF, TGF-β1, or any other compound that increases Ser1177 phosphorylation or Thr495 dephosphorylation or both. Nitric oxide is synthesized from L-arginine by nitric oxide synthase located in endothelial and other cells. Nitric oxide can also be generated by application of various nitric oxide donors such as sodium nitroprusside, nitroglycerin, SIN-1, isosorbid mononitrate, isosorbid dinitrate, and the like. Accordingly, compounds that increase (e.g., by at least 10%, 20%, 30%, 40%, 50%, 60%, 70%, 80%, 90% or more) the level or biological activity of NOS can optionally be administered in combination with L-arginine or a nitric oxide donor (e.g., sodium nitroprusside, nitroglycerin, isosorbidmononitrate, and isosorbo dinitrate). NOS activity can be assayed by standard methods known in the art, including but not limited to the citrulline assay and other assays described in U.S. Patent Application Publication No. 20050256199, the entire disclosure of which is herein incorporated by reference. The Thr495 residue of eNOS is located within the calmodulin (CaM)-binding domain of eNOS. Agonist-induced dephosphorylation of eNOS at Thr495 increases the binding of CaM to the enzyme (Fleming et al., Circ Res. 2001, 88: E68-75), thereby increasing its calcium sensitivity and activation. In addition to TGF-β1 described herein, other agonists that have been shown to cause Thr495 dephosphorylation of eNOS including bradykinin, histamine and VEGF. Thr495 dephosphorylation can be enhanced by the protein kinase C (PKC) inhibitor Ro 31-8220 (Calbiochem) or after PKC downregulation using phorbol 12-myristate 13-acetate (PMA) (Sigma Aldrich). Moreover, agonist-induced dephosphorylation of Thr495 has been shown to be Ca²⁺/calmodulin-dependent and inhibitable by calyculin A (Sigma Aldrich), a protein phosphatase 1 (PP1) inhibitor (Fleming I, et al., Circ Res. 2001, 88: E68-75). Additional compounds that effect eNOS dephosphorylation at Thr495 include histamine and bradykinin (Sigma Aldrich).

Therapeutic Compounds that Increase the Levels or Biological Activity of PGI₂

Prostacyclin is a member of the family of lipid molecules known as eicosanoids. It is produced in endothelial cells from prostaglandin H2 (PGH2) by the action of the enzyme prostacyclin synthase. PGI₂ biological activity includes inhibition of platelet aggregation, relaxation of smooth muscle, reduction of systemic and pulmonary vascular resistance by direct vasodilation, and natriuresis in kidney.

PGI₂ is an anti-thrombotic factor that is stimulated by both VEGF and TGF-β1. PGI₂ biological activity includes inhibition of platelet aggregation and relaxation of vascular smooth muscle and assays for PGI₂ biological activity include any platelet aggregation assay or other PGI2 assay known in the art such as those described in Jakubowski et al., Prostaglandins 47:404 (1994). The invention features the use of any compound that increases (e.g., by at least 10%, 20%, 30%, 40%, 50%, 60%, 70%, 80%, 90% or more) the level or activity of PGI₂, as measured by standard assays known in the art including but not limited to PGI₂ mimetics, iloprost, cicaprost, and aspirin. Additional compounds are known in the art and examples are described in U.S. Pat. No. 5,910,482, the entire disclosure of which is herein incorporated by reference.

### Purified Proteins

For any of the purified proteins, or fragment thereof, the proteins are prepared using standard methods known in the art. Analogs or homologs of any of the therapeutic proteins described above are also included and can be constructed, for example, by making various substitutions of residues or sequences, deleting terminal or internal residues or sequences not needed for biological activity, or adding terminal or internal residues which may enhance biological activity. Amino acid substitutions, deletions, additions, or mutations can be made to improve expression, stability, or solubility of the protein in the various expression systems. Generally, substitutions are made conservatively and take into consideration the effect on biological activity. Mutations, deletions, or additions in nucleotide sequences constructed for expression of analog proteins or fragments thereof must, of course, preserve the reading frame of the coding sequences and preferably will not create complementary regions that could hybridize to produce secondary mRNA structures such as loops or hairpins which would adversely affect translation of the mRNA.

Any of the therapeutic compounds of the invention (e.g., polypeptide, antibodies, small molecule compounds) can also include any modified forms. Examples of post-translational modifications include but are not limited to phosphorylation, glycosylation, hydroxylation, sulfation, acetylation, isoprenylation, proline isomerization, subunit dimerization or multimerization, and cross-linking or attachment to any other proteins, or fragments thereof, or membrane components, or fragments thereof (e.g., cleavage of the protein from the membrane with a membrane lipid component attached). Modifications that provide additional advantages such as increased affinity, decreased off-rate, solubility, stability and in vivo or in vitro circulating time of the polypeptide, or decreased immunogenicity and include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, Creighton, “Proteins: Structures and Molecular Properties,” 2d Ed., W. H. Freeman and Co., N.Y., 1992; “Postranslational Covalent Modification of Proteins,” Johnson, ed., Academic Press, New York, 1983; Seifter et al., Meth. Enzymol., 182:626-646, 1990; Rattan et al., Ann. NY Acad. Sci., 663:48-62, 1992) are also included. The peptidyl therapeutic compound of the invention can also include sequence variants of any of the compounds such as variants that include 1, 2, 3, 4, 5, greater than 5, or greater than 10 amino acid alterations such as substitutions, deletions, or insertions with respect to wild type sequence. Additionally, the therapeutic compound of the invention may contain one or more non-classical amino acids. Non-classical amino acids include, but are not limited to, to the D-isomers of the common amino acids, 2,4-diaminobutyric acid, α-amino isobutyric acid, 4-aminobutyric acid, Abu, 2-amino butyric acid, g-Abu, e-Ahx, 6-amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, β-alanine, fluoro-amino acids, designer amino acids such as β-methyl amino acids, Ca-methyl amino acids, Na-methyl amino acids, and amino acid analogs in general. Furthermore, the amino acid can be D (dextrorotary) or L (levorotary).

Additional post-translational modifications encompassed by the invention include, for example, e.g., N-inked or O-linked carbohydrate chains, processing of N-terminal or C-terminal ends), attachment of chemical moieties to the amino acid backbone, chemical modifications of N-linked or O-linked carbohydrate chains, and addition or deletion of an N-terminal methionine residue.

In addition, chemically modified derivatives of the therapeutic compounds described herein, which may provide additional advantages such as increased solubility, stability and circulating time of the polypeptide, or decreased immunogenicity (see U.S. Pat. No. 4,179,337) are also included. The chemical moieties for derivitization may be selected from water soluble polymers such as, for example, polyethylene glycol, ethylene glycol/propylene glycol copolymers, carboxymethylcellulose, dextran, polyvinyl alcohol and the like. The compound may be modified at random positions within the molecule, or at predetermined positions within the molecule and may include one, two, three or more attached chemical moieties.

The polymer may be of any molecular weight, and may be branched or unbranched. For polyethylene glycol, the preferred molecular weight is between about 1 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog). As noted above, the polyethylene glycol may have a branched structure. Branched polyethylene glycols are described, for example, in U.S. Pat. No. 5,643,575; Morpurgo et al., Appl. Biochem. Biotechnol. 56:59-72, (1996); Vorobjev et al., Nucleosides Nucleotides 18:2745-2750, (1999); and Caliceti et al., Bioconjug. Chem. 10:638-646, (1999), the disclosures of each of which are incorporated by reference.

Any of the therapeutic compounds of the present invention (e.g., polypeptide, antibodies, or small molecule compounds) may also be modified in a way to form a chimeric molecule comprising the therapeutic compound fused to another, heterologous polypeptide or amino acid sequence, such as an Fc sequence, a detectable label, or an additional therapeutic molecule. In one example, an anti-soluble endoglin antibody can be a peptide fused to an Fc fusion protein.

For any of the polypeptides, including antibodies, that are used in the methods of the invention, the nucleic acids encoding the polypeptides or antibodies, or fragments thereof, are also useful in the methods of the invention using standard techniques for gene therapy known in the art and described herein. The invention also includes mimetics, based on modeling the 3-dimensional structure of a polypeptide or peptide fragment and using rational drug design to provide potential inhibitor compounds with particular molecular shape, size and charge characteristics. Following identification of a therapeutic compound, suitable modeling techniques known in the art can be used to study the functional interactions and design mimetic compounds which contain functional groups arranged in such a manner that they could reproduced those interactions. The designing of mimetics to a known pharmaceutically active compound is a known approach to the development of pharmaceuticals based on a lead compound. This might be desirable where the active compound is difficult or expensive to synthesize or where it is unsuitable for a particular method of administration, e.g. peptides are not well suited as active agents for oral compositions as they tend to be quickly degraded by proteases in the alimentary canal. Mimetic design, synthesis and testing may be used to avoid randomly screening large number of molecules for a target property. The mimetic or mimetics can then be screened to see whether they reduce or inhibit soluble endoglin levels or biological activity and further optimization or modification can then be carried out to arrive at one or more final mimetics for in vivo or clinical testing.

Therapeutic Nucleic Acids

Recent work has shown that the delivery of nucleic acid (DNA or RNA) capable of expressing an endothelial cell mitogen such as VEGF to the site of a blood vessel injury will induce proliferation and reendothelialization of the injured vessel. While the present invention does not relate to blood vessel injury, these general techniques for the delivery of nucleic acid to endothelial cells can be used in the present invention for the delivery of nucleic acids encoding soluble endoglin binding proteins, such as TGF-β1, TGF-β3, activin-A, BMP2 and BMP7, or eNOS. The techniques can also be used for the delivery of nucleic acids encoding proteins, such as those described above, known to inhibit the activity of any MMP, cathepsin, or elastase involved in the cleavage and release of soluble endoglin, for the treatment or prevention of pre-eclampsia or eclampsia in a subject. These general techniques are described in U.S. Pat. Nos. 5,830,879 and 6,258,787 and are incorporated herein by reference.

In the present invention the nucleic acid may be any nucleic acid (DNA or RNA) including genomic DNA, cDNA, and mRNA, encoding a soluble endoglin binding proteins such as TGF-β1, TGF-β3, activin-A, BMP2 and BMP7, or eNOS. The nucleic acids encoding the desired protein may be obtained using routine procedures in the art, e.g. recombinant DNA, PCR amplification.

### Modes for Delivering Nucleic Acids

For any of the nucleic acid applications described herein, standard methods for administering nucleic acids can be used. Examples are described in U.S. Patent Application Publication No. 20060067937 and PCT Publication No. WO 06/034507.

Therapeutic Nucleic Acids that Inhibit Soluble Endoglin Expression

The present invention also features the use of antisense nucleobase oligomers to downregulate expression of soluble endoglin mRNA directly. By binding to the complementary nucleic acid sequence (the sense or coding strand), antisense nucleobase oligomers are able to inhibit protein expression presumably through the enzymatic cleavage of the RNA strand by RNAse H. Preferably the antisense nucleobase oligomer is capable of reducing soluble endoglin protein expression in a cell that expresses increased levels of soluble endoglin. Preferably the decrease in soluble endoglin protein expression is at least 10% relative to cells treated with a control oligonucleotide, preferably 20% or greater, more preferably 40%, 50%, 60%, 70%, 80%, 90% or greater. Methods for selecting and preparing antisense nucleobase oligomers are well known in the art. For an example of the use of antisense nucleobase oligomers to down-regulate VEGF expression see U.S. Pat. No. 6,410,322, incorporated herein by reference. Methods for assaying levels of protein expression are also well known in the art and include western blotting, immunoprecipitation, and ELISA.

The present invention also features the use of RNA interference (RNAi) to inhibit expression of soluble endoglin. RNA interference (RNAi) is a recently discovered mechanism of post-transcriptional gene silencing (PTGS) in which double-stranded RNA (dsRNA) corresponding to a gene or mRNA of interest is introduced into an organism resulting in the degradation of the corresponding mRNA. In the RNAi reaction, both the sense and anti-sense strands of a dsRNA molecule are processed into small RNA fragments or segments ranging in length from 21 to 23 nucleotides (nt) and having 2-nucleotide 3′ tails. Alternatively, synthetic dsRNAs, which are 21 to 23 nt in length and have 2-nucleotide 3′ tails, can be synthesized, purified and used in the reaction. These 21 to 23 nt dsRNAs are known as “guide RNAs” or “short interfering RNAs” (siRNAs).

The siRNA duplexes then bind to a nuclease complex composed of proteins that target and destroy endogenous mRNAs having homology to the siRNA within the complex. Although the identity of the proteins within the complex remains unclear, the function of the complex is to target the homologous mRNA molecule through base pairing interactions between one of the siRNA strands and the endogenous mRNA. The mRNA is then cleaved approximately 12 nt from the 3′ terminus of the siRNA and degraded. In this manner, specific genes can be targeted and degraded, thereby resulting in a loss of protein expression from the targeted gene. siRNAs can also be chemically synthesized or obtained from a company that chemically synthesizes siRNAs (e.g., Dharmacon Research Inc., Pharmacia, or ABI).

The specific requirements and modifications of dsRNA are described in PCT Publication No. WO01/75164, and in U.S. Patent Application Publication No. 20060067937 and PCT Publication No. WO 06/034507, incorporated herein by reference.

Soluble Endoglin Based Therapeutic Compounds Useful in Early Pregnancy

Inhibition of full-length endoglin signaling has been shown to enhance trophoblast invasiveness in villous explant cultures (Caniggia I et al, Endocrinology, 1997, 138:4977-88). Soluble endoglin is therefore likely to enhance trophoblast invasiveness during early pregnancy. Accordingly, compositions that increase soluble endoglin levels early in pregnancy in a woman who does not have a pregnancy related hypertensive disorder or a predisposition to a pregnancy related hypertensive disorder may be beneficial for enhancing placentation. Examples of compositions that increase soluble endoglin levels include purified soluble endoglin polypeptides, soluble endoglin encoding nucleic acid molecules, and compounds or growth factors that increase the levels or biological activity of soluble endoglin.

### Assays for Gene and Protein Expression

The following methods can be used to evaluate protein or gene expression and determine efficacy for any of the above-mentioned methods for increasing soluble endoglin binding protein levels, or for decreasing soluble endoglin protein levels.

Blood serum from the subject is measured for levels of soluble endoglin, using methods such as ELISA, western blotting, or immunoassays using specific antibodies. Blood serum from the subject can also be measured for levels of TGF-β1, TGF-β3, activin-A, BMP2, BMP7, or any protein ligand known to bind to soluble endoglin. Methods used to measure serum levels of proteins include ELISA, western blotting, or immunoassays using specific antibodies. In addition, in vitro angiogenesis assays can be performed to determine if the subject's blood has converted from an anti-angiogenic state to a pro-angiogenic state. Such assays are described below in Example 4. A result that is diagnostic of pre-eclampsia or eclampsia is considered an increase of at least 10%, 20%, preferably 30%, more preferably at least 40% or 50%, and most preferably at least 60%, 70%, 80%, 90% or more in the levels of soluble endoglin and a result indicating an improvement in the pre-eclampsia or eclampsia is a decrease of at least 10%, 20%, preferably 30%, more preferably at least 40% or 50%, and most preferably at least 60%, 70%, 80%, 90% or more in the levels of soluble endoglin. Alternatively or additionally, a result that is diagnostic of pre-eclampsia or eclampsia is considered a decrease of at least 10%, 20%, preferably 30%, more preferably at least 40% or 50%, and most preferably at least 60%, 70%, 80%, 90% or more in the levels of eNOS, PGI₂, TGF-β1, TGF-β3, activin-A, BMP2, BMP7, or any protein ligand known to bind to soluble endoglin and a result indicating an improvement in the pre-eclampsia or eclampsia is an increase of at least 10%, 20%, preferably 30%, more preferably at least 40% or 50%, and most preferably at least 60%, 70%, 80%, 90% or more in the levels of eNOS, PGI₂, TGF-β1, TGF-β3, activin-A, BMP2, BMP7, or any protein ligand known to bind to soluble endoglin. A result indicating an improvement in the pre-eclampsia or eclampsia can also be considered conversion by at least 10%, preferably 20%, 30%, 40%, 50%, and most preferably at least 60%, 70%, 80%, 90% or more from an anti-angiogenic state to a pro-angiogenic state using the in vitro angiogenesis assay.

Blood serum or urine samples from the subject can also be measured for levels of nucleic acids or polypeptides encoding eNOS, TGF-β1, TGF-β3, activin-A, BMP2, BMP7, or soluble endoglin. There are several art-known methods to assay for gene expression. Some examples include the preparation of RNA from the blood samples of the subject and the use of the RNA for northern blotting, PCR based amplification, or RNAse protection assays. A positive result is considered an increase of at least 10%, 20%, preferably 30%, more preferably at least 40% or 50%, and most preferably at least 60%, 70%, 80%, 90% or more in the levels of soluble endoglin, TGF-β1, TGF-β3, activin-A, BMP2, BMP7 nucleic acids.

### Therapeutic Antibodies

The elevated levels of soluble endoglin found in the serum samples taken from pregnant women suffering from pre-eclampsia suggests that soluble endoglin is acting as a “physiologic sink” to bind to and deplete the trophoblast cells and maternal endothelial cells of functional growth factors required for the proper development and angiogenesis of the fetus or the placenta. The use of compounds, such as antibodies, to bind to soluble endoglin and neutralize the activity of soluble endoglin (e.g., binding to TGF-β1, TGF-3, activin-A, BMP2, BMP7), may help prevent or treat pre-eclampsia or eclampsia, by producing an increase in free TGF-β1, TGF-β3, activin-A, BMP2, and BMP7. Such an increase would allow for an increase in trophoblast proliferation, migration and angiogenesis required for placental development and fetal nourishment, and for systemic maternal endothelial cell health.

The present invention provides antibodies that specifically bind to soluble endoglin. Preferably, the antibodies bind to the extracellular domain of endoglin or to the ligand binding domain. The antibodies are used to neutralize the activity of soluble endoglin and the most effective mechanism is believed to be through direct blocking of the binding sites for TGF-β1, TGF-β3, activin-A, BMP2, or BMP7, however, other mechanisms cannot be ruled out. Preferred antibodies can bind to an epitope (either as a result of linear structure or three dimensional conformation) on human endoglin that includes any one or more of the peptide sequences indicated in bold and underlined in FIG. 30B (e.g., amino acids 40 to 86, 144 to 199, 206 to 222, 289 to 304, or 375 to 381) or to any of the preferred fragments of soluble endoglin (e.g., amino acids 1 to 437, 4 to 437, 40 to 406, or 1 to 587 of human endoglin). Methods for the preparation and use of antibodies for therapeutic purposes are described in several patents including U.S. Pat. Nos. 6,054,297; 5,821,337; 6,365,157; and 6,165,464; U.S. Patent Application Publication No. 2006/0067937; and PCT Publication No. WO 06/034507 and are incorporated herein by reference. Antibodies can be polyclonal or monoclonal; monoclonal humanized antibodies are preferred. The present invention also includes the antibodies that bind to soluble endoglin, including but not limited to those that bind to any one or more of the peptide sequences indicated in bold and underlined in FIG. 30B or to any of the preferred fragments of soluble endoglin (e.g., amino acids 1 to 437, 4 to 437, 40 to 406, or 1 to 587 of human endoglin).

Therapeutic Uses of Antibodies

When used in vivo for the treatment or prevention of pre-eclampsia or eclampsia, the antibodies of the subject invention are administered to the subject in therapeutically effective amounts. Preferably, the antibodies are administered parenterally or intravenously by continuous infusion. The dose and dosage regimen depends upon the severity of the disease, and the overall health of the subject. The amount of antibody administered is typically in the range of about 0.001 to about 10 mg/kg of subject weight, preferably 0.01 to about 5 mg/kg of subject weight.

For parenteral administration, the antibodies are formulated in a unit dosage injectable form (solution, suspension, emulsion) in association with a pharmaceutically acceptable parenteral vehicle. Such vehicles are inherently nontoxic, and non-therapeutic. Examples of such vehicles are water, saline, Ringer's solution, dextrose solution, and 5% human serum albumin. Nonaqueous vehicles such as fixed oils and ethyl oleate may also be used. Liposomes may be used as carriers. The vehicle may contain minor amounts of additives such as substances that enhance isotonicity and chemical stability, e.g., buffers and preservatives. The antibodies typically are formulated in such vehicles at concentrations of about 1 mg/ml to 10 mg/ml.

### Combination Therapies

Optionally, a therapeutic may be administered in combination with any other standard pre-eclampsia or eclampsia therapy; such methods are known to the skilled artisan and include the methods described in U.S. Patent Application Publication Numbers 20040126828, 20050025762, 20050170444, 20060067937, and 20070104707 and PCT Publication Numbers WO 2004/008946, WO 2005/077007, and WO 06/034507.

Desirably, the invention features the use of a combination of any one or more of the therapeutic agents described herein. Given our discovery that soluble endoglin and sFlt-1 may act in concert to induce vascular damage and pregnancy related hypertensive disorders by interfering with TGF-β1 and VEGF signaling pathway respectively, possibly converging on the NOS signaling pathway, desirable therapeutic methods of the invention include the administration of a compound that decrease sFlt-1 levels or activity or increase VEGF or PlGF levels or activity in combination with a compound that decreases soluble endoglin levels or activity or increase TGF-β, NOS, or PGI2 levels or activity. It will be understood by the skilled artisan that any combination of any of the agents can be used for this purpose. For example, an antibody that specifically binds to soluble endoglin can be administered in combination with VEGF. In another example, a compound that increases TGF-β1 levels or activity can be administered in combination with a compound that increases VEGF or PlGF in order to target both the endoglin and the VEGF pathway. Alternatively, a combination of antibodies against both soluble endoglin and sFlt-1 may be used either directly or in an ex vivo approach (e.g., using a column that is lined with anti-soluble endoglin or sFlt-1 and circulating the patient's blood through the column). Any of these combinations can further include the administration of a compound that increases NOS levels or activity, preferably eNOS, in order to regulate the pathway downstream of the respective receptors.

In addition, the invention provides for the use of any chronic hypertension medications used in combination with any of the therapeutic methods described herein. Medications used for the treatment of hypertension during pregnancy include methyldopa, hydralazine hydrochloride, or labetalol. For each of these medications, modes of administration and dosages are determined by the physician and by the manufacturer's instructions.

### Dosages and Modes of Administration

Preferably, the therapeutic is administered either directly or using an ex vivo approach during pregnancy for the treatment or prevention of pre-eclampsia or eclampsia or after pregnancy to treat post-partum pre-eclampsia or eclampsia. Techniques and dosages for administration vary depending on the type of compound (e.g., chemical compound, purified protein, antibody, antisense, RNAi, or nucleic acid vector) and are well known to those skilled in the art or are readily determined.

Therapeutic compounds of the present invention may be administered with a pharmaceutically acceptable diluent, carrier, or excipient, in unit dosage form. Administration may be parenteral, intravenous, subcutaneous, oral or local by direct injection into the amniotic fluid. Intravenous delivery by continuous infusion is the preferred method for administering the therapeutic compounds of the present invention. The therapeutic compound may be in form of a solution, a suspension, an emulsion, an infusion device, or a delivery device for implantation, or it may be presented as a dry powder to be reconstituted with water or another suitable vehicle before use.

The composition can be in the form of a pill, tablet; capsule, liquid, or sustained release tablet for oral administration; or a liquid for intravenous, subcutaneous or parenteral administration; or a polymer or other sustained release vehicle for local administration.

Methods well known in the art for making formulations are found, for example, in “Remington: The Science and Practice of Pharmacy” (20th ed., ed. A. R. Gennaro A R., 2000, Lippincott Williams & Wilkins, Philadelphia, Pa.). Formulations for parenteral administration may, for example, contain excipients, sterile water, saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Nanoparticulate formulations (e.g., biodegradable nanoparticles, solid lipid nanoparticles, liposomes) may be used to control the biodistribution of the compounds. Other potentially useful parenteral delivery systems include ethylene-vinyl acetate copolymer particles, osmotic pumps, implantable infusion systems, and liposomes. The concentration of the compound in the formulation varies depending upon a number of factors, including the dosage of the drug to be administered, and the route of administration.

The compound may be optionally administered as a pharmaceutically acceptable salt, such as non-toxic acid addition salts or metal complexes that are commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acid such as hydrochloric acid, hydrobromic acid, sulfuric acid phosphoric acid, or the like. Metal complexes include zinc, iron, and the like.

Formulations for oral use include tablets containing the active ingredient(s) in a mixture with non-toxic pharmaceutically acceptable excipients. These excipients may be, for example, inert diluents or fillers (e.g., sucrose and sorbitol), lubricating agents, glidants, and anti-adhesives (e.g., magnesium stearate, zinc stearate, stearic acid, silicas, hydrogenated vegetable oils, or talc).

Formulations for oral use may also be provided as chewable tablets, or as hard gelatin capsules wherein the active ingredient is mixed with an inert solid diluent, or as soft gelatin capsules wherein the active ingredient is mixed with water or an oil medium.

The dosage and the timing of administering the compound depends on various clinical factors including the overall health of the subject and the severity of the symptoms of pre-eclampsia. In general, once pre-eclampsia or a predisposition to pre-eclampsia is detected, continuous infusion of the purified protein is used to treat or prevent further progression of the condition. Treatment can be continued for a period of time ranging from 1 to 100 days, more preferably 1 to 60 days, and most preferably 1 to 20 days, or until the completion of pregnancy. Dosages vary depending on each compound and the severity of the condition and are titrated to achieve a steady-state blood serum concentration ranging from 10 to 20 ng/ml soluble endoglin; and/or 1 to 500 pg/mL free VEGF or free PlGF, or both, preferably 1 to 100 pg/mL, more preferably 5 to 50 pg/mL and most preferably 5 to 10 pg/mL VEGF or PlGF, or 1-5 ng of sFlt-1.

The diagnostic methods described herein can be used to monitor the pre-eclampsia or eclampsia during therapy or to determine the dosages of therapeutic compounds. In one example, a therapeutic compound is administered and the PAAI is determined during the course of therapy. If the PAAI is less than 20, preferably less than 10, then the therapeutic dosage is considered to be an effective dosage. In another example, a therapeutic compound is administered and the soluble endoglin anti-angiogenic index is determined during the course of therapy. If the soluble endoglin anti-angiogenic index is less than 200, preferably less than 100, then the therapeutic dosage is considered to be an effective dosage.

### Subject Monitoring

The disease state or treatment of a subject having pre-eclampsia, eclampsia, or a predisposition to such a condition can be monitored using the diagnostic methods, kits, and compositions of the invention. For example, the expression of a soluble endoglin polypeptide present in a bodily fluid, such as blood, serum, urine, plasma, amniotic fluid, or CSF, can be monitored. The soluble endoglin monitoring can be combined with methods for monitoring the expression of an sFlt-1, VEGF, or PlGF, TGF-β, or eNOS polypeptide or nucleic acid, or PGI₂. Such monitoring may be useful, for example, in assessing the efficacy of a particular drug in a subject or in assessing disease progression. Therapeutics that decrease the expression or biological activity of a soluble endoglin nucleic acid molecule or polypeptide are taken as particularly useful in the invention.

Screening Assays

As discussed above, the level of a soluble endoglin nucleic acid or polypeptide is increased in a subject having pre-eclampsia, eclampsia, or a predisposition to such conditions. Based on these discoveries, compositions of the invention are useful for the high-throughput low-cost screening of candidate compounds to identify those that modulate the expression of a soluble endoglin polypeptide or nucleic acid molecule whose expression is altered in a subject having a pre-eclampsia or eclampsia.

Any number of methods are available for carrying out screening assays to identify new candidate compounds that alter the expression of a soluble endoglin nucleic acid molecule. Examples are described in detail in U.S. Patent Application Publication No. 20060067937 and PCT Publication No. WO 06/034507.

In one working example, candidate compounds may be screened for those that specifically bind to a soluble endoglin polypeptide. The efficacy of such a candidate compound is dependent upon its ability to interact with such a polypeptide or a functional equivalent thereof. Such an interaction can be readily assayed using any number of standard binding techniques and functional assays such as immunoassays or affinity chromatography based assays (e.g., those described in Ausubel et al., supra). In one embodiment, a soluble endoglin polypeptide is immobilized and compounds are tested for the ability to bind to the immobilized soluble endoglin using standard affinity chromatography based assays. Compounds that bind to the immobilized soluble endoglin can then be eluted and purified and tested further for its ability to bind to soluble endoglin both in vivo and in vitro or its ability to inhibit the biological activity of soluble endoglin.

In another example, a candidate compound is tested for its ability to decrease the biological activity of a soluble endoglin polypeptide by decreasing binding of a soluble endoglin polypeptide and a growth factor, such as TGF-β1, TGF-β3, activin-A, BMP-2 and BMP-7. These assays can be performed in vivo or in vitro and the biological activity of the soluble endoglin polypeptide can be assayed using any of the assays for any of the soluble endoglin activities known in the art or described herein. For example, cells can be incubated with a Smad2/3-dependent reporter construct. If desired, the cells can also be incubated in the presence of TGF-β to enhance the signal on the Smad2/3 dependent reporter construct. The cells can then be incubated in the presence of soluble endoglin which will reduce or inhibit TGF-β-induced activation of the Smad2/3 dependent reporter construct. Candidate compounds can be added to the cell and any compound that results in an increase of TGF-β-induced activation of the Smad2/3 dependent reporter in the soluble endoglin treated cells as compared to cells not treated with the compound, is considered a compound that may be useful for the treatment of pre-eclampsia or eclampsia.

In another example, the TGF-β-induced dephosphorylation of eNOS at Thr495 can also be used as an assay for changes in soluble endoglin biological activity. In this example, cells are incubated in the presence of soluble endoglin, which as shown in the experiments described below, inhibits the TGF-β1 dephosphorylation of Thr495 of eNOS. Candidate compounds are then added to the cells and the phosphorylation state of Thr495 is determined. Any compound that results in an increase of TGF-β-induced activation of Thr495 dephosphorylation in the soluble endoglin treated cells as compared to cells not treated with the compound, is considered a compound that may be useful for the treatment of pre-eclampsia or eclampsia.

### EXAMPLES

The following examples are intended to illustrate the invention. They are not meant to limit the invention in any way.

### Example 1

### Increased Levels of Endoglin mRNA and Protein in Pregnant Women with Pre-Eclampsia

In an attempt to identify novel secreted factors playing a pathologic role in pre-eclampsia, we performed gene expression profiling of placental tissue from 17 pregnant women with pre-eclampsia and 13 normal pregnant women using Affymetrix U95A microarray chips. We found that the gene for endoglin was upregulated in women with pre-eclampsia.

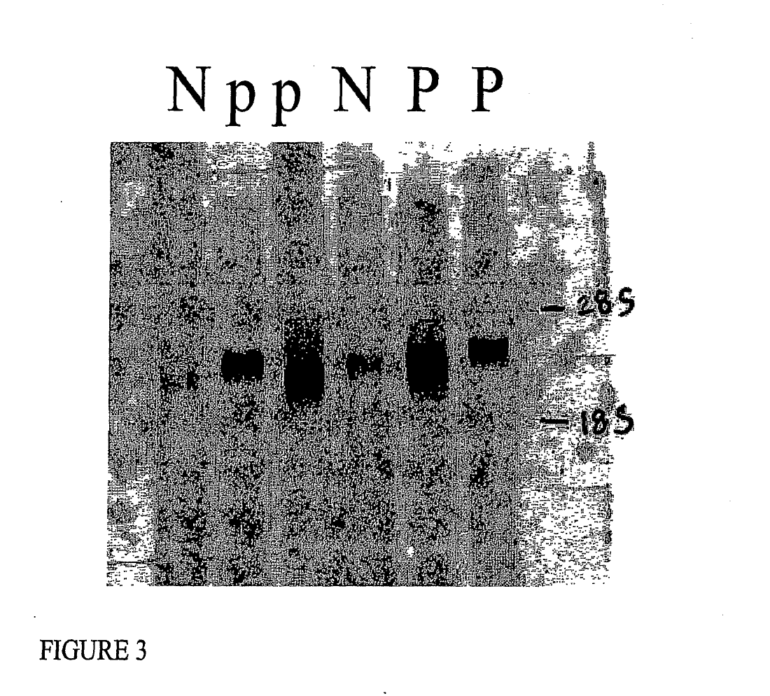

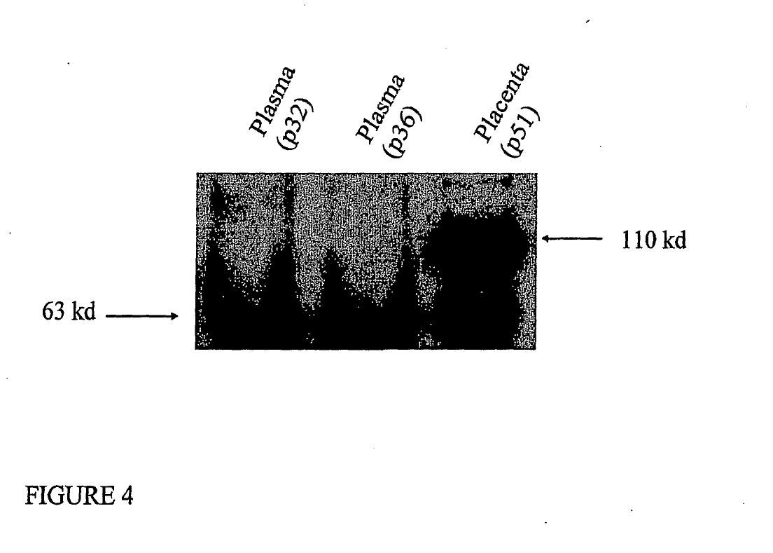

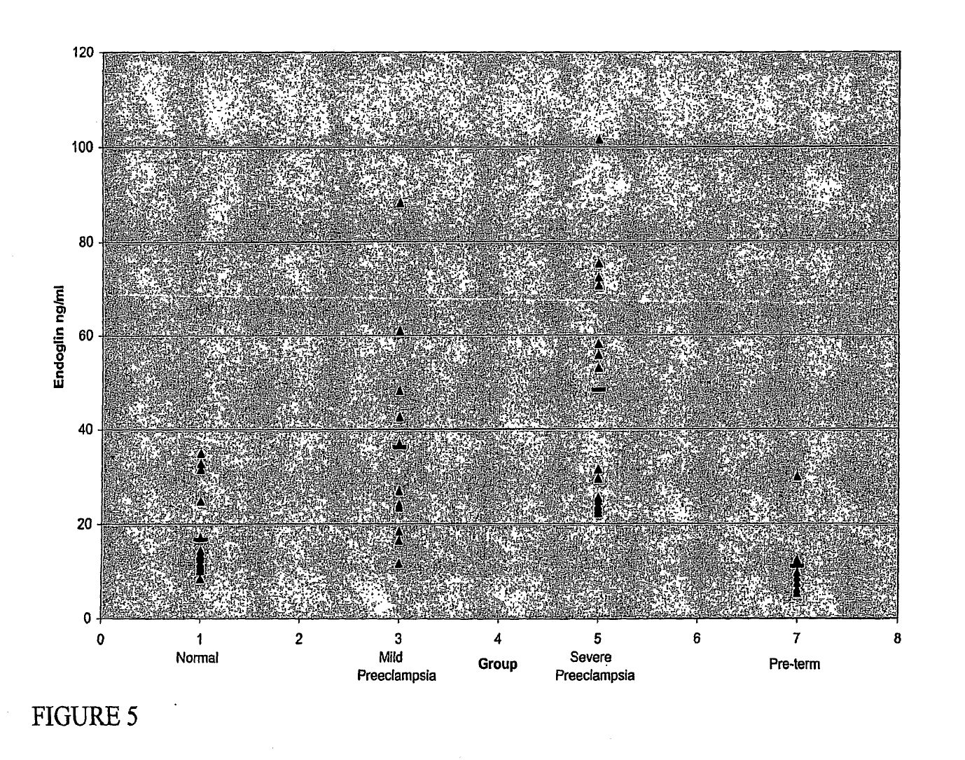

In order to confirm the upregulation of endoglin in pre-eclampsia, we performed Northern blots to analyze the placental endoglin mRNA levels (FIG. 3) and western blot analysis to measure serum protein levels of endoglin (FIG. 4) in pre-eclamptic pregnant women as compared with normotensive pregnant women. Pre-eclampsia was defined as (1) a systolic blood pressure (BP) >140 mmHg and a diastolic BP>90 mmHg after 20 weeks gestation, (2) new onset proteinuria (1+ by dipstik on urinanalysis, >300 mg of protein in a 24 hour urine collection, or random urine protein/creatinine ratio >0.3, and (3) resolution of hypertension and proteinuria by 12 weeks postpartum. Patients with underlying hypertension, proteinuria, or renal disease were excluded. Patients were divided into mild and severe pre-eclampsia based on the presence or absence of nephrotic range proteinuria (>3 g of protein on a 24 hour urine collection or urine protein/creatinine ratio greater than 3.0). The mean urine protein/creatinine ratios in the mild pre-eclampsia group were 0.94+/−0.2 and in the severe pre-eclampsia group were 7.8+/−2.1. The mean gestational ages of the various groups were as follows: normal 38.8+/−0.2 weeks, mild pre-eclampsia 34+/−1.2 weeks, severe pre-eclampsia 31.3+/−0.6 weeks, and pre-term 29.5+/−2.0 weeks. Placental samples were obtained immediately after delivery. Four random samples were taken from each placenta, placed in RNAlater stabilization solution (Ambion, Austin, Tex.) and stored at −70° C. RNA isolation was performed using Qiagen RNAeasy Maxi Kit (Qiagen, Valencia, Calif.).

Northern blots probed with a 400 base pair probe in the coding region of endoglin (Unigene Hs. 76753) corresponding to the N-terminal region (gene bank #BC014271) and an 18S probe as a normalization control showed an increase in placental endoglin mRNA (see Knebelmann et al., Cancer Res. 58:226-231 (1998)). Western blots probed with an antibody to the amino terminus of endoglin showed an increase in both placental and maternal serum levels of endoglin protein in pre-eclamptic pregnant women as compared to normotensive pregnant women.

### Example 2

### Demonstration of a Soluble Endoglin Polypeptide in the Placentas and Serum of Pre-Eclamptic Patients

The western blot analysis used to measure the levels of endoglin protein in placentas and serum from pre-eclamptic women suggested the presence of a smaller protein (approximately 63-65 kDa), that was present in the placenta and serum of pre-eclamptic pregnant women (FIGS. 4 and 30A). We have demonstrated that this smaller fragment is the extracellular domain of endoglin. This truncated version is likely to be shed from the placental syncitiotrophoblasts and endothelial cells and circulated in excess quantities in patients with pre-eclampsia. This soluble form of endoglin may be acting as an anti-angiogenic agent by binding to circulating ligands that are necessary for normal vascular health.

The predicted length of the soluble form of the protein is approximately 437 amino acids (including the peptide leader sequence, 412 amino acids without the leader sequence). sEng was purified from the serum of preeclamptic patients. Fractions 4 and 5 eluted from the 44G4-IgG (anti-Eng) Sepharose, were run on SDS-PAGE under reducing conditions and tested by Western blot using a polyclonal antibody to Eng. The eluted fractions were subjected to mass spectrometry analysis (3 runs) and the peptides identified are shown in (FIG. 30B). The purification and analysis by mass spectrometry revealed several Eng-specific peptides ranging from Gly40 to Arg406 indicating a soluble form (soluble endoglin) corresponding to the N-terminal region of the full-length protein bold on the sequence of human endoglin.

### Example 3

### Circulating Concentrations of Soluble Endoglin in Women with Normal Versus Pre-Eclamptic Pregnancies

In order to compare the levels of circulating, soluble endoglin from the serum of normal, mildly pre-eclamptic, or severely pre-eclamptic women, we performed ELISA analysis on blood samples taken from these women. All the patients for this study were recruited at the Beth Israel Deaconess Medical Center after obtaining appropriate IRB-approved consents. Pre-eclampsia was defined as (1) Systolic BP>140 and diastolic BP>90 after 20 weeks gestation in a previously normotensive patient, (2) new onset proteinuria (1+ by dipstick on urinanalysis or >300 mg of protein in a 24 hr urine collection or random urine protein/creatinine ratio >0.3), and (3) resolution of hypertension and proteinuria by 12 weeks postpartum. Patients with baseline hypertension, proteinuria, or renal disease were excluded. For the purposes of this study, patients were divided into mild and severe pre-eclampsia based on the absence or presence of nephrotic-range proteinuria (>3 g of protein on a 24 hour urine collection or urine protein to creatinine ratio greater than 3.0). HELLP syndrome was defined when patients had evidence of thrombocytopenia (<100000 cells/μl), increased LDH (>600 IU/L) and increased AST (>70 IU/L). Healthy pregnant women were included as controls. 8 patients with pre-term deliveries for other medical reasons were included as additional controls. Placental samples were obtained immediately after delivery. Serum was collected from pregnant patients at the time of delivery (0-12 hours prior to delivery of the placenta) after obtaining informed consent. These experiments were approved by the Institutional Review Board at the Beth Israel Deaconess Medical Center.

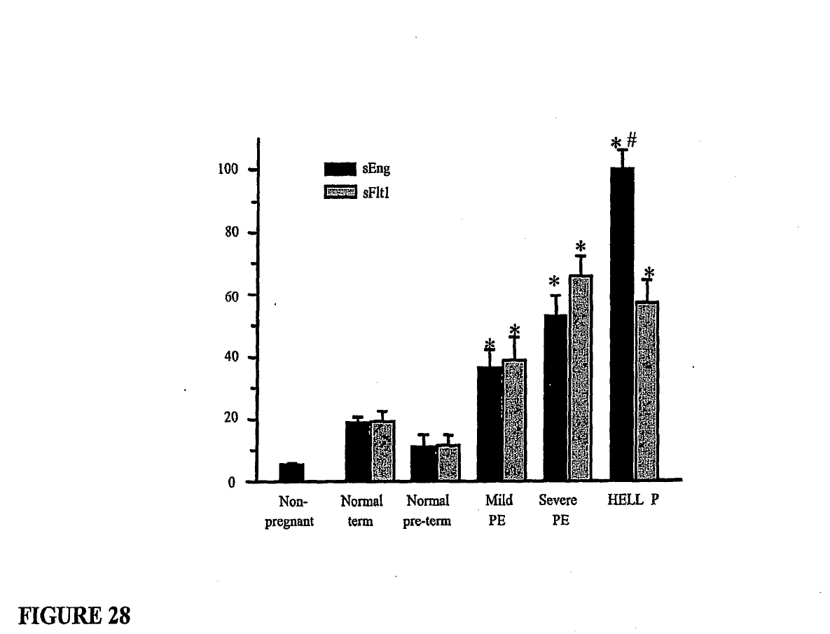

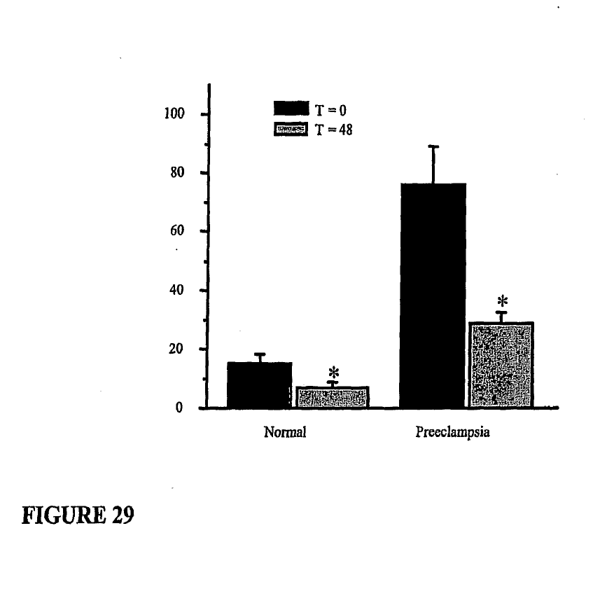

Using the serum specimens from patients described in Table 1, we measured the circulating concentrations of soluble endoglin in the various groups of pre-eclamptic patients and control pregnant patients. When pre-eclamptic patients were further sub-divided into those with and without HELLP, sEng concentrations were three-, five- and ten-fold higher in mild, severe and HELLP syndrome preeclamptics, respectively, compared to gestational age-matched pre-term controls (FIG. 28). Concentrations of sEng in pregnant patients correlated with those of sFlt1 (R²=0.56), except in the HELLP group where sEng was higher than sFlt1. In a subset of patients, blood samples obtained 48 hours after placental delivery showed a 70% reduction in mean sEng circulating levels in preeclamptic and normal pregnant patients (FIG. 29).

TABLE 1
Clinical characteristics and circulating soluble endoglin
in the various patient groups

| | | Mild pre- | Severe pre- | Severe pre- | |
|---|---|---|---|---|---|
| | Normal | eclampsia | eclampsia, no | eclampsia with | Pre-term |
| | (n = 30) | (n = 11) | HELLP (n = 17) | HELLP (n = 11) | (n = 8) |
| Maternal age (yrs) | 32.43 | 33.18 | 29.5 | 33.73 | 31.88 |
| Gestational age (wks) | 38.65 | 31.91* | 29.06* | 26.52* | 30.99* |
| Primiparous (%) | 43.3 | 63.6 | 47.1 | 90.9 | 62.5 |
| Systolic blood pressure (mmHg) | 122 | 157* | 170* | 166* | 123 |
| Diastolic blood pressure (mmHg) | 72 | 99* | 104* | 103* | 77 |
| Proteinuria (g protein/g creatinine) | 0.37 | 2.5* | 8.64* | 5.16* | 0.6 |
| Uric acid (mg/dl) | 5.27 | 6.24 | 7.29* | 6.31 | 7.35 |
| Hematocrit (%) | 35.5 | 33.6 | 33.7 | 33.5 | 34.3 |
| Platelet count | 238 | 230 | 249 | 69.4* | 229 |
| Creatinine (mg/dl) | 0.55 | 0.62 | 0.62 | 0.64 | 0.67 |
| Soluble endoglin in (ng/ml) | 18.73 | 36.12* | 52.55** | 99.83*** | 10.9 |

*P < 0.05,
**P < 0.005

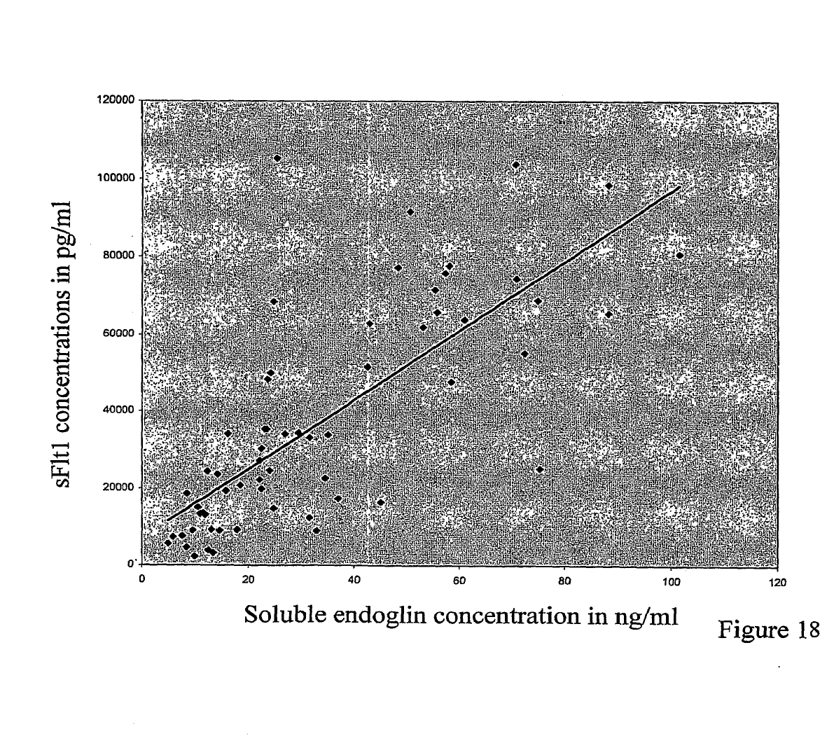

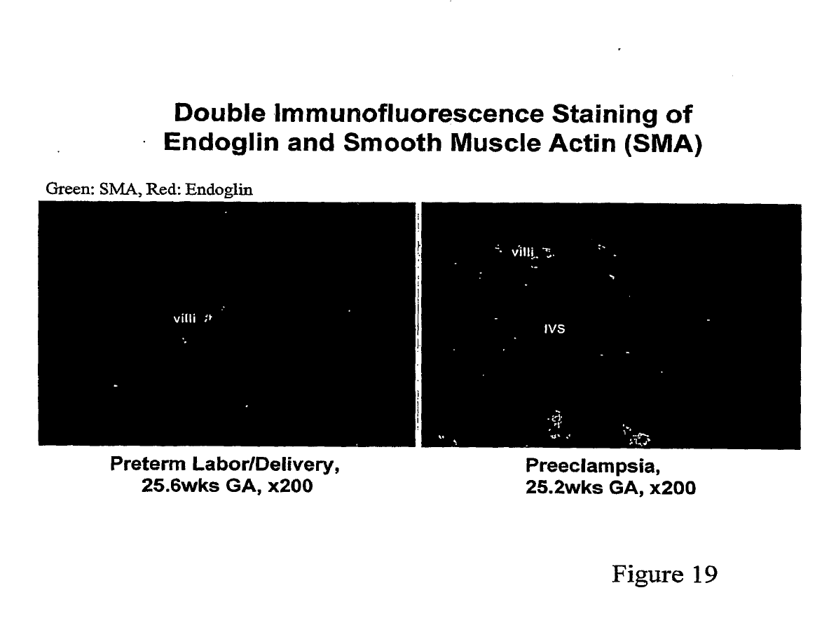

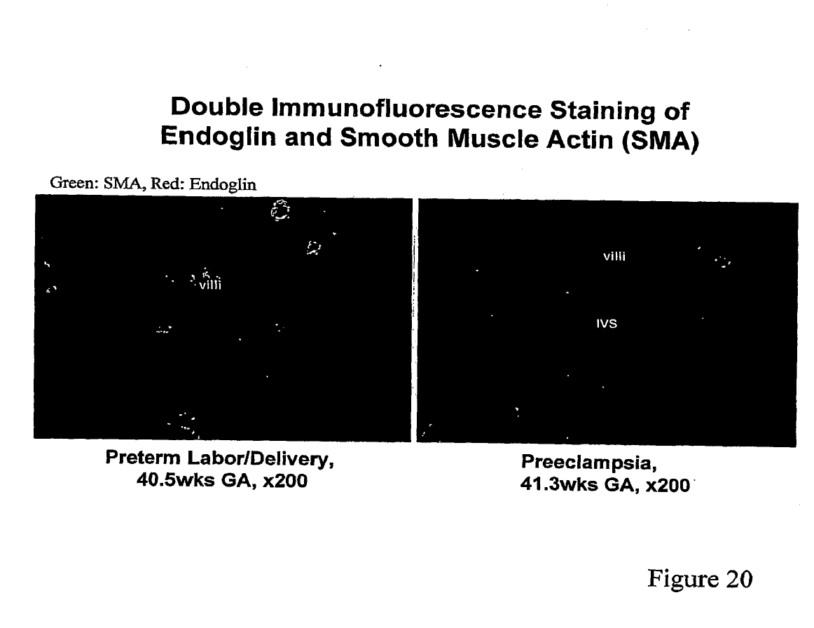

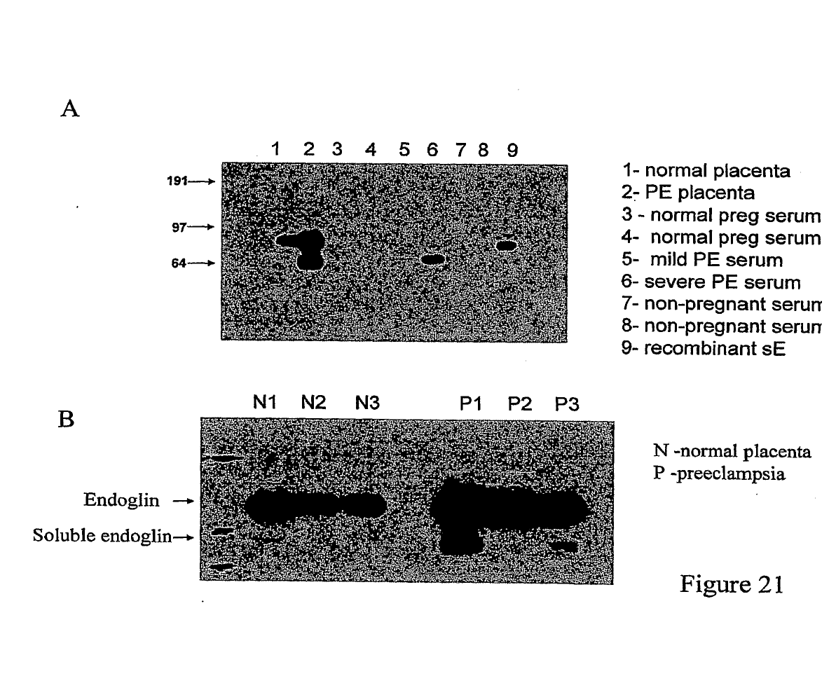

The average serum concentrations of soluble endoglin was at least two fold higher in mild pre-eclampsia and 3-4 fold higher in patients with severe pre-eclampsia. In pre-eclamptic patients complicated with the HELLP syndrome, the concentration of soluble endoglin was at least 5-10 fold higher than gestational age matched control specimens. Additionally, the levels of soluble endoglin in pregnant patients correlate with the levels of sFlt-1 (FIG. 18). The R2 value for correlation was 0.6. (Note that the circulating concentrations of sFlt-1 reported here are at least 4-5 fold higher than previously published (Maynard et al., supra). This is due to a difference in the sensitivity of a new ELISA kit from R&D systems which lacks urea in the assay diluent and therefore gives consistently higher values than previously published.) In other words, patients with the highest levels of soluble endoglin also had the highest circulating levels of sFlt1. The origin of soluble endoglin is most likely the syncitiotrophoblast of the placenta as evidenced by the enhanced staining seen on our placental immunohistochemistry (FIGS. 19 and 20). These figures show that endoglin protein is expressed by the syncitiotrophoblasts and is vastly upregulated in pre-eclampsia. Our western blot data (FIGS. 21A and 21B) and the lack of detectable alternative splice variants by northern blot supports the notion that soluble endoglin is likely a shed form of the extracellular domain of the membrane endoglin protein. It is approximately 65 kDA in size and is produced at elevated levels in pre-eclamptic placentas and it circulates in higher amounts in pre-eclamptic sera. This protein was present at much lower levels in the sera of normal pregnant women and barely detectable in non-pregnant women. Soluble endoglin expression in pre-eclamptic placenta was four-fold higher than in normal pregnancy (n=1−/group, P<0.01). Quantitation of sEng/Eng in these specimens showed no significant difference between normal (0.43) and preeclamptic (0.56) placentae (n=10/group, P=0.4), suggesting that sEng is derived from the full-length protein and that both Eng and sEng are similarly increased in preeclampsia.

The following methods were used for some of the experiments described in this example.

### Immunohistochemistry

Immunohistochemistry on placental samples for endoglin and α-Smooth muscle actin (SMA) was done as reported by (Leach et al., Lancet 360:1215-1219 (2002)). Briefly, the frozen placenta section obtained from patients without preeclampsia (n=10) and with preeclampsia (n=10) slides were incubated with a serum-free protein blocking solution (DAKO) for 30 minutes at room temperature and then with the primary antibody at room temperature (mouse monoclonal anti-Endoglin: 1:50 dilution; DAKO) for 2 hours. The slides were then washed with phosphate buffered saline for 10 minutes. The secondary antibody, Rhodamine conjugated sheep anti-mouse IgG, 1:200 dilution (Biomeda) was applied for 1 hour. Sections were again washed with phosphate buffered saline and subsequently incubated with a 1:400 dilution of FITC-conjugated mouse anti-human SMA (Dako) for 30 minutes at room temperature. Immunoreactivity of Endoglin was reviewed using a SPOT advanced imaging system (RT SLIDER Diagnostic Instruments, Inc) by a pathologist who was blinded to the clinical diagnosis.

### ELISA and Western Blots

ELISA was performed using a commercially available ELISA kit from R & D Systems, MN (for example, Cat # DNDG00) and as previously described (Maynard et al, J. Clin. Invest. 111:649-658, 2003). Western blots were performed essentially as described previously (Maynard et al, supra, and Kuo et al. Proc. Natl. Acad. Sci. 98:4605-4610 (2001))).

Immunoprecipitation (IP) Experiments

IP followed by western blots were used to identify and characterize soluble endoglin in the placental tissue and serum specimens from patients with pre-eclampsia. Human placental tissue was washed with cold PBS and lysed in homogenization buffer [10 mM Tris-HCl, pH 7.4; 15 mM NaCl; 60 mM KCl; 1 mM EDTA; 0.1 mM EGTA; 0.5% Nonidet P-40; 5% sucrose; protease mixture from Roche (Indianapolis, Ind.)] for 10 minutes. Placental lysates were then subjected to immunoprecipitation with an anti-human monoclonal mouse endoglin antibody (mAb P4A4, Santa Cruz Biotechnology, Inc., Santa Cruz, Calif.). Immunoaffinity columns were prepared by the directional coupling of 3-5 mg of the purified antibody to 2 ml protein A-Sepharose using an immunopure IgG orientation kit (Pierce Chemical Co., Rockford, Ill., USA) according to the manufacturer's instructions. Columns were then washed extensively with RIPA buffer containing protease mixture, and bound proteins were eluted with 0.1 mol/L glycine-HCl buffer, pH 2.8. The eluent was collected in 0.5-ml fractions containing 1 mol/L Tris-HCl buffer. Protein-containing fractions were pooled and concentrated 9- to 10-fold with CENTRICON Centrifugal Concentrator (Millipore Corp., Bedford, Mass., USA). The immunoprecipitated samples were separated on a 4-12% gradient gel (Invitrogen) and proteins were transferred to polyvinylidene difluoride (PVDF) membranes. Endoglin protein was detected by western blots using polyclonal anti-human rabbit endoglin primary antibody (H-300, Santa Cruz Biotechnology, Inc., Santa Cruz, Calif.).

### Purification of Soluble Endoglin and Analysis by Mass Spectrometry

Serum (10 ml) from preeclamptic patients was sequentially applied onto CM Affi-gel blue and protein A Sepharose (Bio-Rad) columns to remove albumin and immunoglobulins, respectively. The flow through was slowly applied to a 2.5 ml column of mAb 44G4 IgG to human Eng, conjugated to Sepharose (Gougos et al., Int. Immuno. 4:83-92, (1992)). Bound fractions were eluted with 0.02 M diethylamine pH 11.4 and immediately neutralized with 1 M Tris pH 7.8. Fractions 4 and 5 with elevated absorbance at 280 nm were pooled, reduced with 10 mM DTT for 1 h at 57° C. and alkylated with 0.055 M iodoacetomide. The samples were then completely digested with trypsin (1:100). The lyophilized sample was resuspended in 0.1% tri-fluoroacetic acid and injected in a CapLC (Waters) HPLC instrument. Peptides were separated using a 75 μm Nano Series column (LC Packings) and analyzed using a Qstar XL MS/MS system. The data was searched using the Mascot search engine (Matrix Science) against the human protein database, NCBInr.

### Example 4

### Model Assay for Angiogenesis

An endothelial tube assay can be used an in vitro model of angiogenesis. Growth factor reduced Matrigel (7 mg/mL, Collaborative Biomedical Products, Bedford, Mass.) is placed in wells (100 μl/well) of a pre-chilled 48-well cell culture plate and is incubated at 37° C. for 25-30 minutes to allow polymerization. Human umbilical vein endothelial cells (30,000+ in 300 μl of endothelial basal medium with no serum, Clonetics, Walkersville, Md.) at passages 3-5 are treated with 10% patient serum, plated onto the Matrigel coated wells, and are incubated at 37° C. for 12-16 hours. Tube formation is then assessed through an inverted phase contrast microscope at 4× (Nikon Corporation, Tokyo, Japan) and is analyzed (tube area and total length) using the Simple PCI imaging analysis software.

### Example 5

### Soluble Endoglin Protein Levels as a Diagnostic Indicator of Pre-Eclampsia and Eclampsia in Women (Romero Study)

This study was designed to evaluate whether soluble endoglin is altered during clinical pre-eclampsia and whether it can be used to predict pre-eclampsia and eclampsia in women.

This study was done under collaboration with Dr. Roberto Romero, at the Wayne State University/NICHD Perinatology Branch, Detroit, Mich. A retrospective longitudinal case-control study was conducted using a banked biological sample database as previously described in Chaiworapongsa et al. (The Journal of Maternal-Fetal and Neonatal Medicine, January 2005, 17 (1):3-18). All women were enrolled in the prenatal clinic at the Sotero del Rio Hospital, Santiago, Chile, and followed until delivery. Prenatal visits were scheduled at 4-week intervals in the first and second trimester, and every two weeks in the third trimester until delivery. Plasma samples were selected from each patient only once for each of the following six intervals: (1) 7-16 weeks, (2) 16-24 weeks, (3) 24-28 weeks, (4) 28-32 weeks, (5) 32-37 weeks, and (6) >37 weeks of gestation. For each pre-eclamptic case, one control was selected by matching for gestational age (+/−2 weeks) at the time of clinical diagnosis of pre-eclampsia. The clinical criteria for the diagnosis of pre-eclampsia were the same as previously described in Chaiworapongsa et al, supra.

### Measurement of Plasma Endoglin Levels

The plasma samples stored at −70° C. were thawed and plasma soluble endoglin levels were measured in one batch using the commercially available ELISA kits from R&D systems, Minneapolis, Minn. (Catalog #DNDG00).

### Statistical Analysis

Analysis of covariance was used to assess the difference in plasma concentrations of soluble endoglin between patients destined to develop pre-eclampsia and in normal pregnancy after adjusting for gestational age at blood sampling and intervals of sample storage. Chi-square or Fisher's exact tests were employed for comparisons of proportions. The statistics package used was SPSS V.12 (SPSS Inc., Chicago, Ill.). Significance was assumed for a p value of less than 0.05.

### Results

The clinical characteristics of the study population are described in Table 2. The group with pre-eclampsia included more nulliparous women and delivered earlier than the control group. Importantly, the birth weights of the fetuses were smaller in the pre-eclamptic group and there were a higher proportion of women carrying small-for-gestational-age (SGA) infants.

TABLE 2
Clinical characteristics of the study population

| | Normal | | |
|---|---|---|---|
| | pregnancy | Pre-eclampsia | |
| | n = 44 | n = 44 | P |
| Age (y) | 29 ± 6 | 26 ± 6 | 0.04* |
| Nulliparity | 11 (25%) | 30 (68.2%) | <0.001* |
| Smoking | 10 (22.7%) | 1 (2.3%) | 0.007* |
| GA at delivery (weeks) | 39.7 ± 1.1 | 36.9 ± 2.7 | <0.001* |
| Birthweight (grams) | 3,372 ± 383 | 2,710 ± 766 | <0.001* |
| Birthweight <10^{th} percentile | 0 | 16 (36.4%) | <0.001* |

Value expressed as mean ± sd or number (percent)
GA: gestational age

The clinical characteristics of patients with pre-eclampsia are described in Table 3. Thirty-two (72%) of the patients had severe pre-eclampsia, while 10 patients had severe early-onset pre-eclampsia defined as onset <34 weeks.

TABLE 3
Clinical characteristics of patients with pre-eclampsia

| Blood pressure (mmHg) | |
|---|---|
| Systolic | 155 ± 15 |
| Diastolic | 100 ± 8 |
| Mean arterial pressure | 118 ± 9 |
| Proteinuria (dipstick) | 3 ± 0.8 |
| Aspartate aminotransferase^{α} (SGOT) (U/L) | 29 ± 31 |
| Platelet count^{β} (×10³) (μ/L) | 206 ± 59 |
| Severe pre-eclampsia | 32 (72.7%) |
| GA at pre-eclampsia diagnosed ≦34 weeks | 10 (22.7%) |
| GA at pre-eclampsia diagnosed ≧37 weeks | 27 (61.4%) |

Value expressed as mean ± sd or number (percent)
^{α}(n = 26);
^{β}(n = 42)

The serum soluble endoglin levels in the controls and the pre-eclamptic women measured in the 6 gestational age windows are shown in Table 4. Amongst the pre-eclamptics, their specimens were divided into two groups—clinical pre-eclampsia (samples taken at the time of symptoms of pre-eclampsia) and preclinical pre-eclampsia (samples taken prior to clinical symptoms). The data shows that at mid-pregnancy (24-28 weeks of gestation), serum soluble endoglin concentrations start rising in women destined to develop pre-eclampsia and become at least 3 fold higher than controls by 28-32 weeks of gestation. Blood samples taken from women with clinical pre-eclampsia show a very dramatic (nearly 10-15 fold) elevation when compared to gestational age matched controls.

TABLE 4
Plasma soluble endoglin concentrations in normal pregnancy and pre-eclampsia

| | | | Pre-clinical | | | |
|---|---|---|---|---|---|---|
| | Normal | | samples | | Clinical samples | |
| | pregnancy | p | Pre-eclampsia | p | Pre-eclampsia | p^{β} |
| 1^{st} blood sampling (7.1-16 weeks) | | | | | | |
| Soluble Endoglin (ng/ml) | 3.89 ± .928 | 0.9 | 3.96 ± 1.28 | | | |
| Gestational age (weeks) | 12.3 ± 2.2 | 0.2 | 11.6 ± 2.4 | | | |
| Range | 8.4-15.9 | | 7.7-15.1 | | | |
| | n = 37 | | n = 34 | | | |
| 2^{nd} blood sampling (16.1-24 weeks) | | | | | | |
| Soluble Endoglin (ng/ml) | 3.36 ± 1.11 | 0.1 | 3.79 ± 1.37 | | | |
| Gestational age (weeks) | 19.4 ± 1.7 | 0.06 | 20.2 ± 2.1 | | | |
| Range | 16.3-23.4 | | 16.7-24.0 | | | |
| | n = 44 | | n = 36 | | | |
| 3^{rd} blood sampling (24.1-28 weeks) | | | | | | |
| Soluble Endoglin (ng/ml) | 3.18 ± .729 | 0.009* | 5.27 ± 4.12 | | | |
| Gestational age (weeks) | 25.9 ± 1.3 | 0.2 | 26.4 ± 1.1 | | | |
| Range | 24.1-28.0 | | 24.6-28.0 | | | |
| | n = 38 | | n = 29 | | | |
| 4^{th} blood sampling (28.1-32 weeks) | | | | | | |
| Soluble Endoglin (ng/ml) | 3.7 ± 1.1 | <0.001* | 10.2 ± 9.8 | 0.01* | 96.1 ± 25.7 | 0.05 |
| Gestational age (weeks) | 29.9 ± 1.1 | 1.0 | 30.2 ± 1.0 | 1.0 | 30.4 ± 1.4 | 1.0 |
| Range | 28.3-32.0 | | 28.7-32.0 | | 29.4-31.4 | |
| | n = 42 | | n = 33 | | n = 2^{δ} | |
| 5^{th} blood sampling (32.1-36.9 | | | | | | |
| weeks) | | | | | | |
| Soluble Endoglin (ng/ml) | 5.79 ± 2.42 | 0.003* | 10.51 ± 6.59 | <0.001* | 43.14 ± 25.6 | <0.001* |
| Gestational age (weeks) | 34.7 ± 1.3 | 1.0 | 34.8 ± 1.5 | 1.0 | 34.5 ± 1.2 | 1.0 |
| Range | 32.4-36.6 | | 32.6-36.7 | | 32.6-36.6 | |
| | n = 37 | | n = 20 | | n = 13 | |
| 6^{th} blood sampling (>=37 weeks) | | | | | | |
| Soluble Endoglin (ng/ml) | 8.9 ± 4.5 | | — | | 15.23 ± 10.61 | 0.006* |
| Gestational age (weeks) | 39.4 ± 1.0 | | | | 38.8 ± 1.1 | 0.05 |
| Range | 37.0-40.7 | | | | 37.6-41.4 | |
| | n = 27 | | | | n = 27 | |

p^{β}: compared between samples at clinical manifestation of pre-eclampsia and normal pregnancy
Value expressed as mean ± sd
^{δ}2 pre-eclamptic patients had no blood samples available at clinical manifestation

To examine the relationship between plasma soluble endoglin concentrations and the interval to clinical diagnosis of pre-eclampsia, plasma samples of pre-eclamptic patients at different gestational ages were stratified according to the interval from blood sampling to clinical diagnosis into five groups: (1) at clinical diagnosis, (2) 2-5.9 weeks before clinical manifestation, (3) 6-10.9 weeks before clinical manifestation, (4) 11-15.9 weeks before clinical manifestation, and (5) 16-25 weeks before clinical manifestation. The data shown in Table 5 demonstrates that the plasma soluble endoglin levels start going up at 6-10.9 weeks before onset of symptoms in pre-eclamptics and are at least 3 fold higher at 2-5.9 weeks before symptoms in women destined to develop pre-eclampsia.

TABLE 5
Plasma soluble endoglin concentrations in
normal and pre-eclamptic pregnant women.

| | | | |
|---|---|---|---|
| Blood sampling | Normal pregnancy | Pre-eclampsia | P |

At clinical manifestation

| | | | |
|---|---|---|---|
| Soluble Endoglin (ng/ml) | 7.63 ± 4.22 | 27.72 ± 26.20 | <0.001* |
| Gestational age (weeks) | 37.2 ± 3.0 | 37.1 ± 2.7 | 0.9 |
| Range | 28.9-40.7 | 29.4-41.4 | |
| | n = 42 | n = 42^{δ} | |

2-5.9 weeks before clinical manifestation

| | | | |
|---|---|---|---|
| Soluble Endoglin (ng/ml) | 4.67 ± 2.32 | 15.07 ± 10.15 | <0.001* |
| Gestational age (weeks) | 31.6 ± 3.8 | 32.8 ± 2.8 | 0.2 |
| Range | 24.1-36.3 | 27.1-36.7 | |
| | n = 27 | n = 27 | |
| Interval before clinical | | 3.8 ± 1.1 | |
| manifestation (weeks) | | | |

6-10.9 weeks before clinical manifestation

| | | | |
|---|---|---|---|
| Soluble Endoglin (ng/ml) | 3.61 ± 1.05 | 5.89 ± 3.07 | <0.001* |
| Gestational age (weeks) | 28.5 ± 2.9 | 28.5 ± 2.9 | 0.9 |
| Range | 19.7-32.6 | 19.6-34.4 | |
| | n = 37 | n = 37 | |
| Interval before clinical | | 8.3 ± 1.4 | |
| manifestation (weeks) | | | |

11-15.9 weeks before clinical manifestation

| | | | |
|---|---|---|---|
| Soluble Endoglin (ng/ml) | 3.35 ± 0.77 | 3.57 ± 0.92 | 0.5 |
| Gestational age (weeks) | 24.5 ± 3.1 | 24.2 ± 3.3 | 0.8 |
| Range | 17.6-27.9 | 17.7-28.0 | |
| | n = 19 | n = 19 | |
| Interval before clinical | | 13.2 ± 1.3 | |
| manifestation (weeks) | | | |

16-25 weeks before clinical manifestation

| | | | |
|---|---|---|---|
| Soluble Endoglin (ng/ml) | 3.44 ± 1.07 | 3.69 ± 1.18 | 0.3 |
| Gestational age (weeks) | 17.6 ± 3.5 | 16.5 ± 4.5 | 0.2 |
| Range | 9.1-23.4 | 8.0-22.7 | |
| | n = 42 | n = 42 | |
| (Interval before clinical | | 20.6 ± 3.6 | |
| manifestation weeks) | | | |

Value expressed as mean ± sd
^{δ}2 pre-eclamptic patients had no blood samples available at clinical manifestation

To examine the diagnostic potential of plasma soluble endoglin concentrations to identify those destined to develop pre-eclampsia, patients were stratified into early onset pre-eclampsia (PE<34 weeks) and late onset pre-eclampsia (PE>34 weeks). For patients with early-onset pre-eclampsia, the mean plasma soluble endoglin levels was significantly higher in pre-eclampsia (before clinical diagnosis) than in normal pregnancy starting around 16-24 weeks of gestation (Table 6) with very dramatic differences in 24-28 week and 28-32 week gestational windows. In contrast, for patients with late-onset pre-eclampsia, plasma soluble endoglin concentrations in pre-clinical pre-eclampsia was significantly higher than in normal pregnancy only at 28-32 weeks with very dramatic differences at 32-36 week of gestation (Table 7).

TABLE 6
Plasma soluble endoglin concentrations in normal pregnant women and patients
who developed clinical Pre-eclampsia at 34 weeks of gestation or less.

| | Normal | | Pre-clinical samples | | Clinical samples | |
|---|---|---|---|---|---|---|
| | pregnancy | p | Pre-eclampsia | p | pre-eclampsia^{δ} | p^{β} |
| 1^{st} blood sampling (7.1-16 weeks) | | | | | | |
| Soluble Endoglin (ng/ml) | 3.89 ± .928 | 0.7 | 3.81 ± 1.11 | | | |
| Gestational age (weeks) | 12.3 ± 2.2 | 0.4 | 11.6 ± 2.6 | | | |
| Range | 8.4-15.9 | | 8.0-15.1 | | | |
| | n = 37 | | n = 8 | | | |
| 2^{nd} blood sampling (16.1-24 weeks) | | | | | | |
| Soluble Endoglin (ng/ml) | 3.36 ± 1.11 | 0.02* | 4.60 ± 1.72 | | | |
| Gestational age (weeks) | 19.4 ± 1.7 | 0.7 | 19.8 ± 2.9 | | | |
| Range | 16.3-23.4 | | 17.3-23.9 | | | |
| | n = 44 | | n = 7 | | | |
| 3^{rd} blood sampling (24.1-28 weeks) | | | | | | |
| Soluble Endoglin (ng/ml) | 3.189 ± .729 | <0.001* | 10.22 ± 6.17 | | | |
| Gestational age (weeks) | 25.9 ± 1.3 | 0.03* | 26.8 ± 0.6 | | | |
| Range | 24.1-28.0 | | 26.0-27.3 | | | |
| | n = 38 | | n = 6 | | | |
| 4^{th} blood sampling (28.1-32 weeks) | | | | | | |
| Soluble Endoglin (ng/ml) | 3.70 ± 1.10 | 0.01* | 17.66 ± 8.9 | 0.008* | 96.10 ± 25.76 | 0.05 |
| Gestational age (weeks) | 29.9 ± 1.1 | 1.0 | 29.7 ± 1.1 | 1.0 | 30.4 ± 1.4 | 1.0 |
| Range | 28.3-32.0 | | 28.7-31.3 | | 29.4-31.4 | |
| | n = 42 | | n = 6 | | n = 2^{δ} | |
| 5^{th} blood sampling (32.1-36.9 weeks) | | | | | | |
| Soluble Endoglin (ng/ml) | 5.79 ± 2.42 | | | | 53.38 ± 32.09 | 0.001* |
| Gestational age (weeks) | 34.7 ± 1.3 | | | | 33.5 ± 0.5 | <0.001* |
| Range | 32.4-36.6 | | | | 32.6-34.0 | |
| | n = 37 | | | | n = 6 | |

p^{β}: compared between samples at clinical manifestation of pre-eclampsia and normal pregnancy
Value expressed as mean ± sd
^{δ}2 pre-eclamptic patients had no blood samples available at clinical manifestation

TABLE 7
Plasma soluble endoglin concentrations in normal pregnant women and pre-
eclamptics (34 weeks of gestation)

| | | | | | Clinical | |
|---|---|---|---|---|---|---|
| | Normal | | Pre-clinical samples | | samples | |
| | pregnancy | p | Pre-eclampsia | p | Pre-eclampsia | p^{β} |
| 1^{st} blood sampling (7.1-16 weeks) | | | | | | |
| Soluble Endoglin (ng/ml) | 3.89 ± .928 | 0.9 | 4.01 ± 1.35 | | | |
| Gestational age (weeks) | 12.3 ± 2.2 | 0.2 | 11.6 ± 2.4 | | | |
| Range | 8.4-15.9 | | 7.7-15.1 | | | |
| | n = 37 | | n = 26 | | | |
| 2^{nd} blood sampling (16.1-24 weeks) | | | | | | |
| Soluble Endoglin (ng/ml) | 3.36 ± 1.11 | 0.4 | 3.59 ± 1.23 | | | |
| Gestational age (weeks) | 19.4 ± 1.7 | 0.04* | 20.3 ± 1.9 | | | |
| Range | 16.3-23.4 | | 16.7-24.0 | | | |
| | n = 44 | | n = 29 | | | |
| 3^{rd} blood sampling (24.1-28 weeks) | | | | | | |
| Soluble Endoglin (ng/ml) | 3.18 ± .729 | 0.1 | 3.98 ± 2.13 | | | |
| Gestational age (weeks) | 25.9 ± 1.3 | 0.4 | 26.3 ± 1.1 | | | |
| Range | 24.1-28.0 | | 24.6-28.0 | | | |
| | n = 38 | | n = 23 | | | |
| 4^{th} blood sampling (28.1-32 weeks) | | | | | | |
| Soluble Endoglin (ng/ml) | 3.70 ± 1.10 | 0.001* | 8.57 ± 9.45 | | | |
| Gestational age (weeks) | 29.9 ± 1.1 | 0.2 | 30.3 ± 1.0 | | | |
| Range | 28.3-32.0 | | 28.7-32.0 | | | |
| | n = 42 | | n = 27 | | | |
| 5^{th} blood sampling (32.1-36.9 weeks) | | | | | | |
| Soluble Endoglin (ng/ml) | 5.79 ± 2.42 | <0.001* | 10.51 ± 6.59 | <0.001* | 34.36 ± 16.30 | <0.001* |
| Gestational age (weeks) | 34.7 ± 1.3 | 1.0 | 34.8 ± 1.5 | 0.9 | 35.4 ± 0.9 | 0.7 |
| Range | 32.4-36.6 | | 32.6-36.7 | | 34.3-36.6 | |
| | n = 37 | | n = 20 | | n = 7 | |
| 6^{th} blood sampling (>=37 weeks) | | | | | | |
| Soluble Endoglin (ng/ml) | 8.98 ± 45.12 | | — | | 15.23 ± 10.61 | 0.006* |
| Gestational age (weeks) | 39.4 ± 1.0 | | | | 38.8 ± 1.1 | 0.05 |
| Range | 37.0-40.7 | | | | 37.6-41.4 | |
| | n = 27 | | | | n = 27 | |

p^{β}: compared between samples at clinical manifestation of pre-eclampsia and normal pregnancy
Value expressed as mean ± sd

### Summary

The results of these experiments demonstrate that women with clinical pre-eclampsia have very high levels of circulating soluble endoglin when compared to gestational age matched controls. The results also demonstrate that women destined to develop pre-eclampsia (pre-clinical pre-eclampsia) have higher plasma soluble endoglin levels than those who are predicted to have a normal pregnancy. The increase in soluble endoglin levels is detectable at least 6-10 weeks prior to onset of clinical symptoms. Finally, these results demonstrate that both early onset and late onset pre-eclampsia have elevated circulating soluble endoglin concentrations, but the alterations are more dramatic in the early onset pre-eclampsia.

### Example 6

### Soluble Endoglin Protein Levels as a Diagnostic Indicator of Pre Eclampsia and Eclampsia in Women (CPEP Study)

As described above, we have discovered that soluble endoglin, a cell surface receptor for the pro-angiogenic protein TGF-β and expressed on endothelium and syncytiotrophoblast, is upregulated in pre-eclamptic placentas. In the experiments described above, we have shown that in pre-eclampsia excess soluble endoglin is released from the placenta into the circulation through shedding of the extracellular domain; soluble endoglin may then synergize with sFlt1, an anti-angiogenic factor which binds placental growth factor (PlGF) and VEGF, to cause endothelial dysfunction. To test this hypothesis, we compared serum concentrations of soluble endoglin, sFlt1, and free PlGF throughout pregnancy in women who developed pre-eclampsia and in those women with other pregnancy complication such as gestational hypertension (GH) and pregnancies complicated by small-for-gestational (SGA) infants to those of women with normotensive control pregnancies. This study was done in collaboration with the Dr. Richard Levine at the NIH.

There were two principal objectives of this study. The first objective was to determine whether, in comparison with normotensive controls, elevated serum concentrations of soluble endoglin, sFlt1, and reduced levels of PlGF can be detected before the onset of pre-eclampsia and other gestational disorders such as gestational hypertension or pregnancies complicated by small-for-gestational (SGA) infants. The second objective was to describe the time course of maternal serum concentrations of soluble endoglin, sFlt-1, and free PlGF with respect to gestational age in women with pre-eclampsia, gestational hypertension, or SGA with separate examination of specimens obtained before and after onset of clinical symptoms, and in normotensive controls.

### Methods

Clinical Information

This study was a case control study of pregnancy complications (premature pre-eclampsia, term pre-eclampsia, gestational hypertension, pregnancies with SGA infants, normotensive control pregnancies) nested within the cohort of 4,589 healthy nulliparous women who participated in the Calcium for Pre-eclampsia Prevention trial (CPEP). 120 random cases were selected from each of the study groups. The study methods were identical to the nested case control study recently performed for pre-eclampsia (Levine et al, N. Eng. J. Med. 2004, 350:672-83). From each woman blood specimens were obtained before study enrollment (13-21 wks), at 26-29 weeks, at 36 weeks, and on suspicion of hypertension or proteinuria. All serum specimens collected at any time during pregnancy before onset of labor and delivery were eligible for the study. Cases included 120 women who developed term pre-eclampsia, gestational hypertension, or SGA and who delivered a liveborn or stillborn male baby without known major structural or chromosomal abnormalities, and from whom a baseline serum specimen was obtained. For premature pre-eclampsia, defined as (PE<37 weeks) all 72 patients from the CPEP cohort were studied. The clinical criterion for the diagnosis of pre-eclampsia is described in Levine et al., (2004), supra. All cases of gestational hypertension were required to have a normal urine protein measurement within the interval from 1 day prior to onset of gestational hypertension through 7 days following. SGA was defined as <10th and <5th (severe SGA) percentile, using Zhang & Bowes' tables of birthweight for gestational age, specific for race, nulliparity, and infant gender. Controls were randomly selected from women without pre-eclampsia or gestational hypertension or SGA who delivered a liveborn or stillborn baby without known major structural malformations or chromosomal anomalies and matched, one control to one case, by the clinical center, gestational age at collection of the first serum specimen (±1 wk), by freezer storage time (±1 year), and by number of freeze-thaws. A total of 1674 serum specimens were studied. Matching by gestational age was done to control for gestational age-related differences in levels of sFlt-1, VEGF, and PlGF. Matching for freezer storage time was done to minimize differences due to possible degradation during freezer storage. Matching by clinical center was done to control for the fact that pre-eclampsia rates differed significantly between centers, perhaps due to differences in the pathophysiology of the disease. In addition, the centers may have used slightly different procedures for collecting, preparing, and storing specimens. Matching by number of thaws was also performed to ensure that cases and controls will have been subjected equally to freeze thaw degradation.

ELISA Measurements

ELISA for the various angiogenic markers were performed at the Karumanchi laboratory by a single research assistant that was blinded to the clinical outcomes.

Commercially available ELISA kits for soluble endoglin (DNDG00), sFlt1 (DVR100), PlGF (DPG00) were obtained from R&D systems, (Minneapolis, Minn.).

Statistical Analysis

T-test was used for the comparison of the various measurements after logarithm tic transformation to determine significance. P<0.05 was considered as statistically significant.

### Results

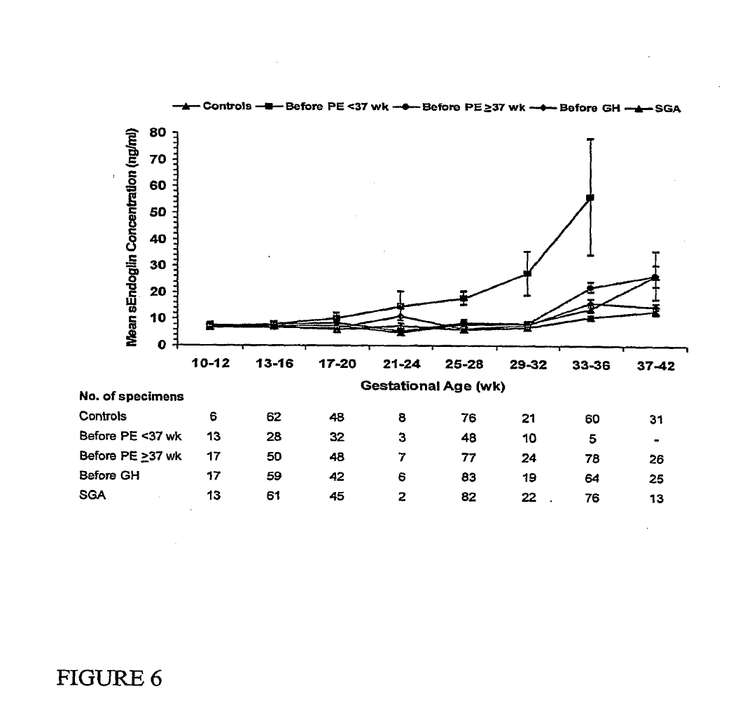

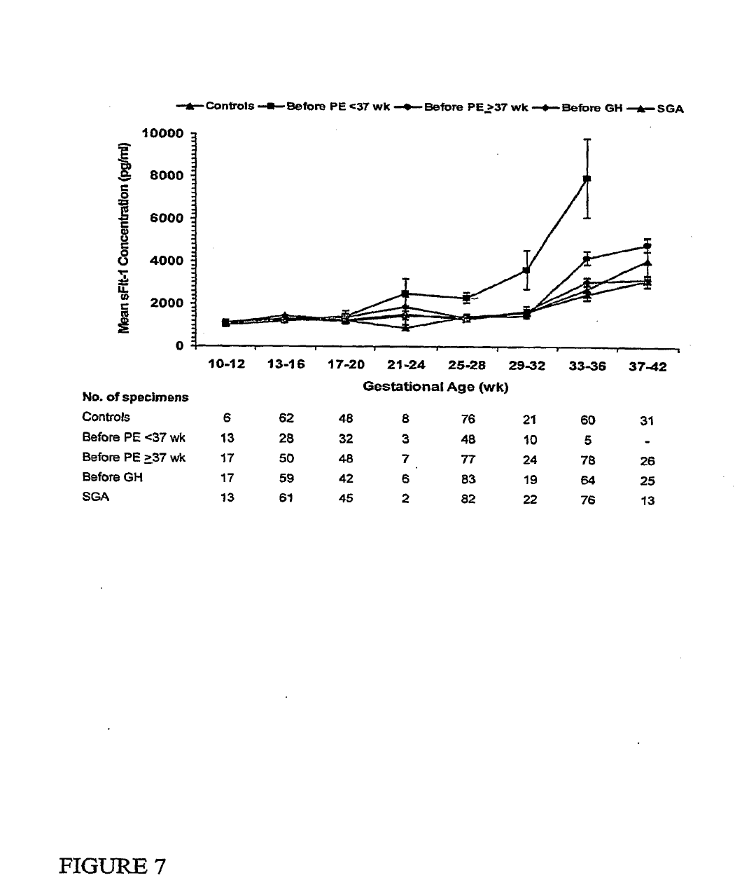

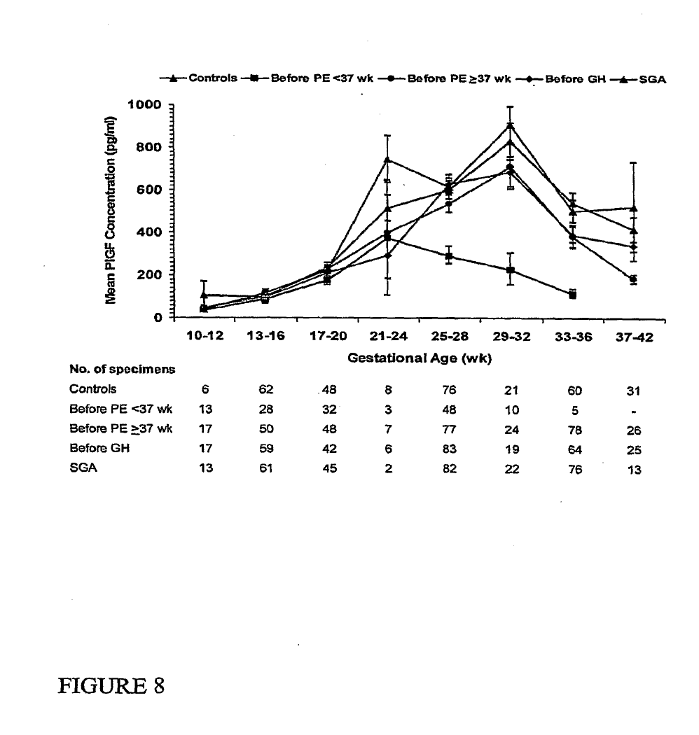

The mean soluble endoglin (FIG. 6), sFlt1 (FIG. 7) and PlGF (FIG. 8) concentrations for the five different study groups of pregnant women throughout pregnancy during the various gestational age group windows as described in the methods are shown in FIGS. 6-8. For the pre-eclampsia groups and gestational hypertensive groups, specimens taken after onset of clinical symptoms are not shown here. Compared with gestational age-matched control specimens, soluble endoglin and sFlt1 increased and free PlGF decreased beginning 9-11 weeks before preterm pre-eclampsia, reaching levels 5-fold (46.4 vs 9.8 ng/ml, P<0.0001) and 3-fold higher (6356 vs 2316 pg/ml, P<0.0001) and 4-fold lower (144 vs 546 pg/ml, P<0.0001), respectively, after pre-eclampsia onset. For term pre-eclampsia, soluble endoglin increased beginning 12-14 weeks, free PlGF decreased beginning 9-11 weeks, and sFlt1 increased <5 weeks before pre-eclampsia onset. Serum concentrations of sFlt1 and free PlGF did not differ significantly between pregnancies with SGA or average for gestation age/large for gestation age (AGA/LGA) infants from 10-42 weeks of gestation. Serum soluble endoglin was modestly increased in SGA pregnancies beginning at 17-20 weeks (7.2 vs 5.8 ng/ml, P=0.03), attaining concentrations of 15.7 and 43.7 ng/ml at 37-42 weeks for mild and severe SGA, respectively, as compared with 12.9 ng/ml in AGA/LGA pregnancies (severe SGA vs AGA/LGA, P=0.002). In the gestational hypertension study, compared with GA-matched control specimens, modest increases in soluble endoglin were apparent <1-5 weeks before gestational hypertension, reaching levels 2-fold higher for soluble endoglin (29.7 vs 12.5 ng/ml, P=0.002) after onset of gestational hypertension. The adjusted odds ratio for subsequent preterm PE for specimens obtained at 21-32 weeks which were in the highest quartile of control soluble endoglin concentrations (>7.2 ng/ml), as compared to all other quartiles, was 9.8 (95% CI 4.5-21.5).

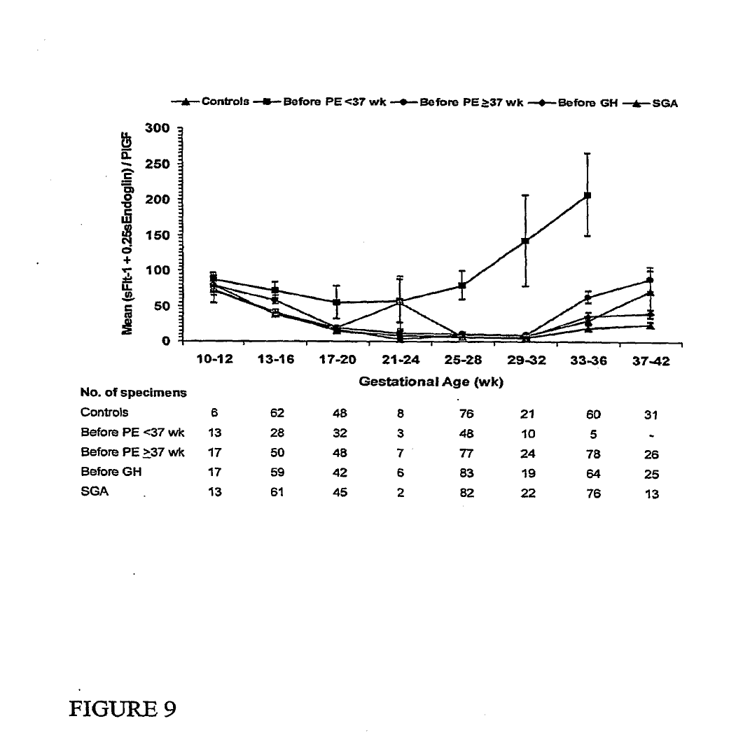

The soluble endoglin anti-angiogenic index for pre-eclampsia was defined as (sFlt1+0.25 soluble endoglin)/PlGF. The index was calculated throughout the various gestational age groups for the five different study groups. The soluble endoglin anti-angiogenic index for pre-eclampsia anti-angiogenesis for samples taken prior to clinical symptoms is shown in FIG. 9. Elevated values for the soluble endoglin anti-angiogenic index were noted as early as 17-20 weeks of pregnancies and seemed to get more dramatic with advancing gestation in severe pre-mature pre-eclampsia. In term pre-eclampsia, SGA and GH, there was a modest elevation during the end of pregnancy (33-36 weeks) when compared to control women.

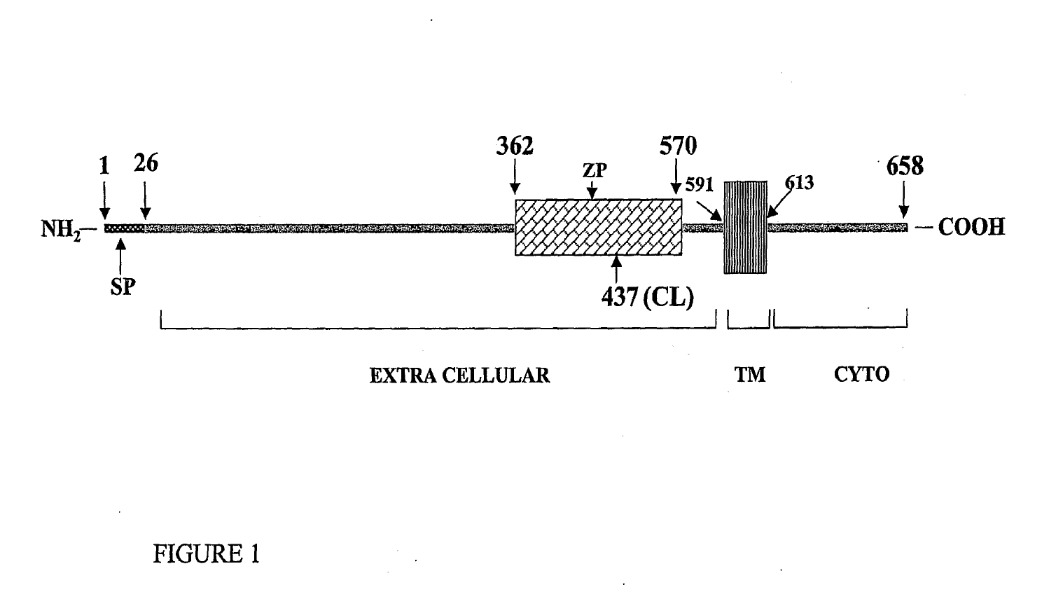

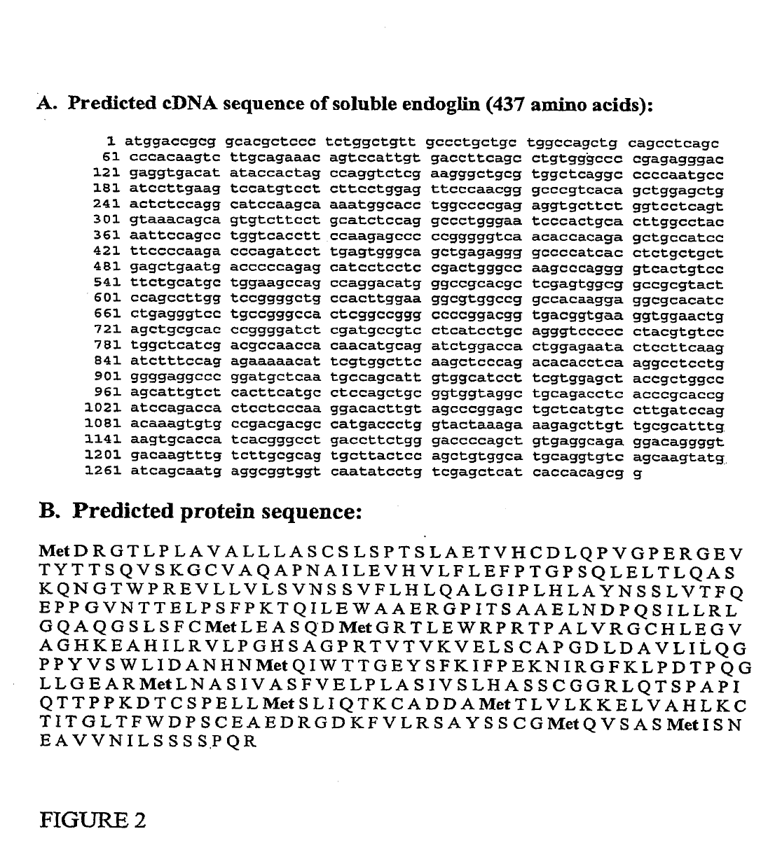

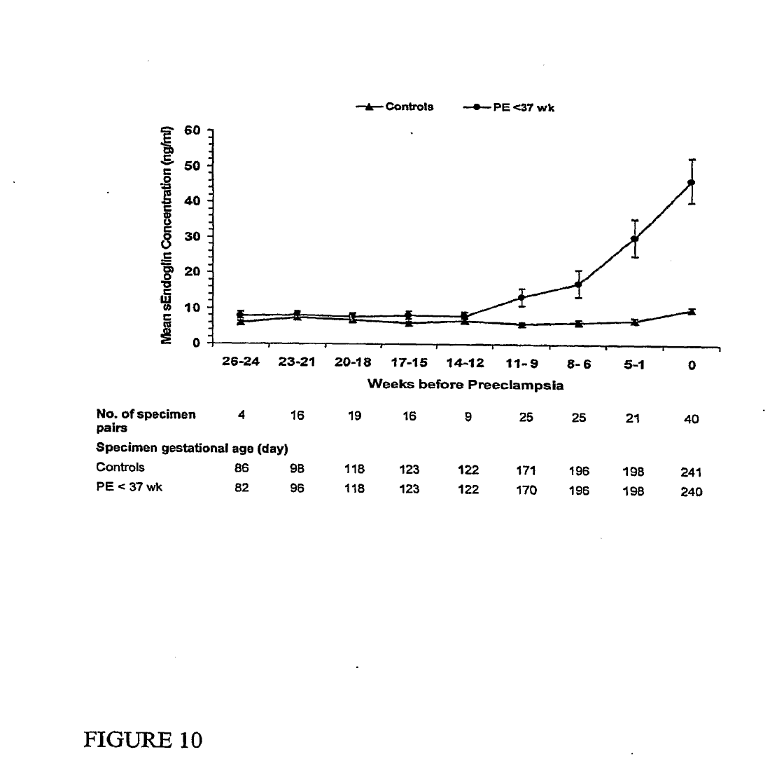

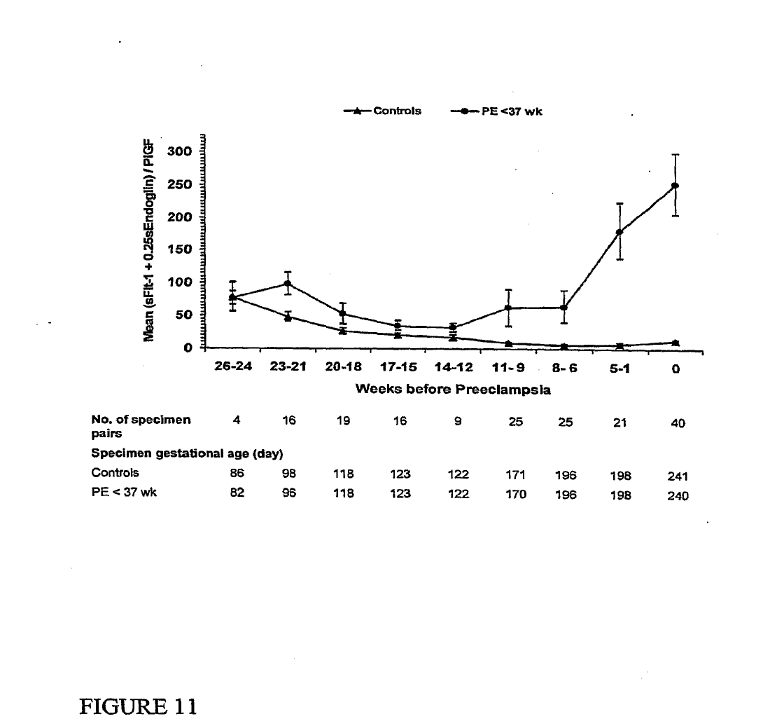

FIGS. 10 and 11 depict the mean concentrations of soluble endoglin (FIG. 10) and soluble endoglin anti-angiogenic index (FIG. 1) according to the number of weeks before clinical premature pre-eclampsia (PE<37 weeks). Even as early 9-11 weeks prior to the onset of premature pre-eclampsia, there was a 2-3 fold elevation in soluble endoglin and soluble endoglin anti-angiogenic index in women destined to develop pre-eclampsia with dramatic elevations (>5 fold) in 1-5 weeks preceding clinical symptoms.

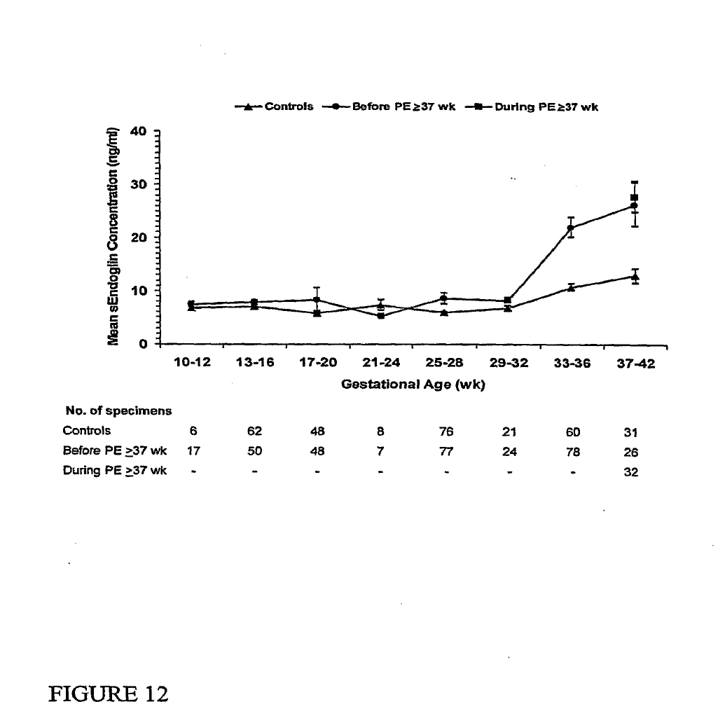

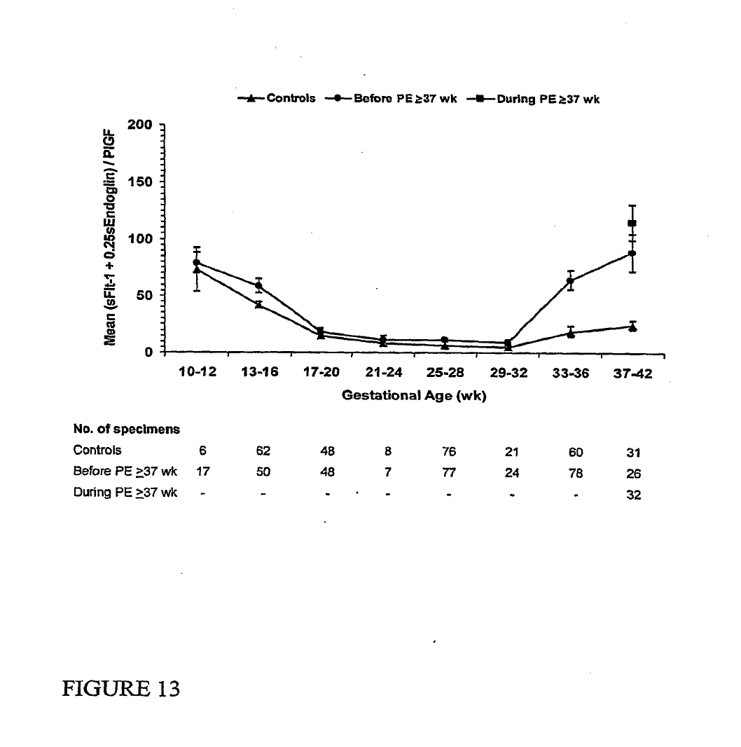

FIGS. 12 and 13 show the alteration in soluble endoglin (FIG. 12) and the soluble endoglin anti-angiogenic index (FIG. 13) throughout pregnancy for term pre-eclampsia (PE>37 weeks) before and after symptoms. Elevation in soluble endoglin and the soluble endoglin anti-angiogenic index are noted starting at 33-36 weeks of pregnancy reaching on average 2-fold higher levels at the time of clinical pre-eclampsia.

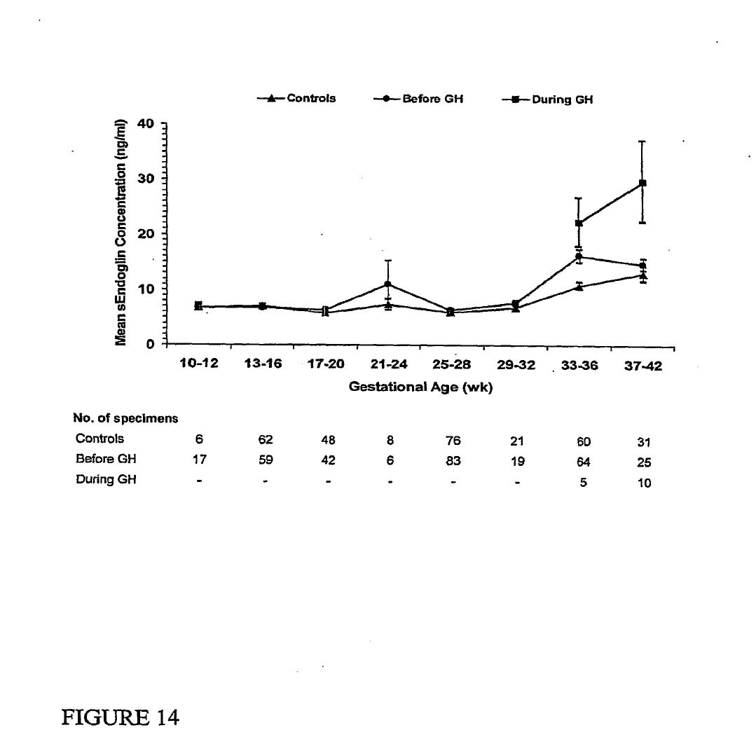

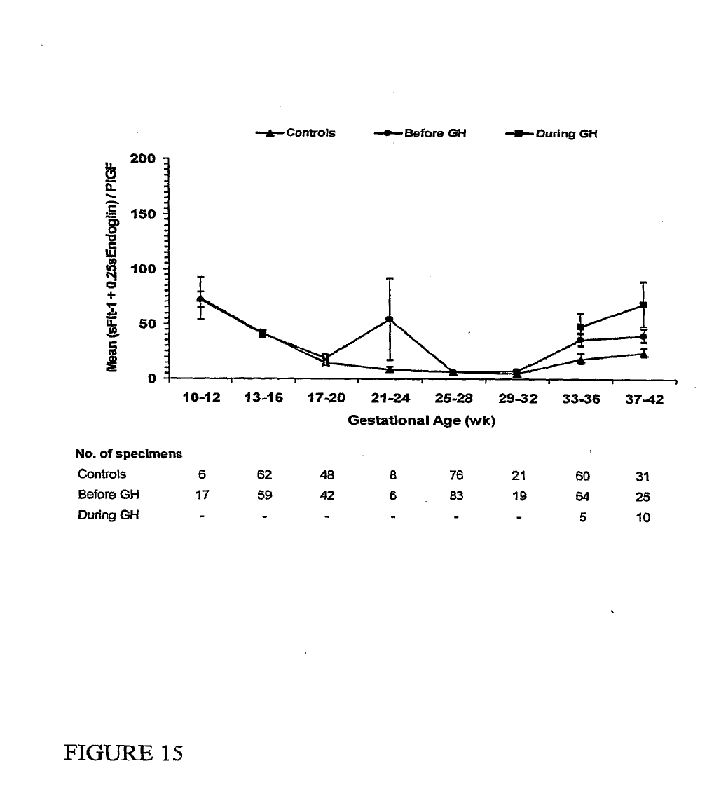

FIGS. 14 and 15 show a modest elevation in soluble endoglin (FIG. 14) and the soluble endoglin anti-angiogenic index (FIG. 15) detected in women during gestational hypertension, and 1-5 weeks preceding gestational hypertension (during 33-36 week of pregnancy) when compared to normotensive controls.

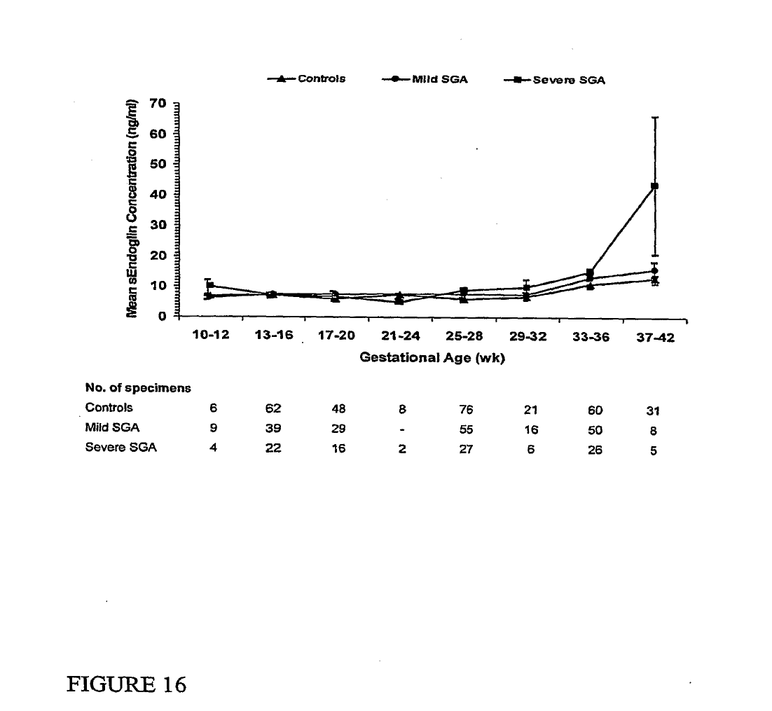

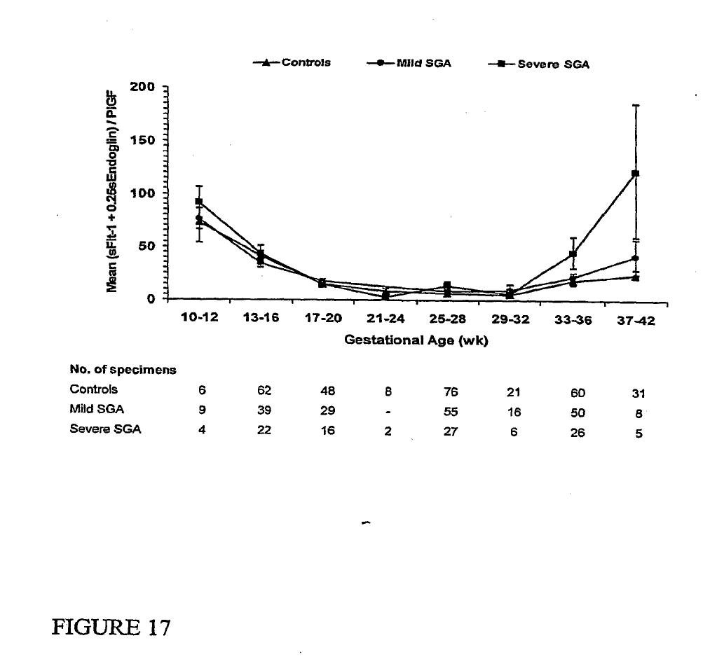

FIGS. 16 and 17 show a modest elevations in soluble endoglin (FIG. 16) and the soluble endoglin anti-angiogenic index (FIG. 17) detected during the 33-36 week gestational windows in women with severe SGA and not in all women with SGA when compared to control pregnancies.

### Summary

The results of this study show that the soluble endoglin levels and soluble endoglin anti-angiogenic index levels, when measured prior to 33 weeks of pregnancy, was dramatically elevated in women destined to develop premature pre-eclampsia and in women with clinical premature pre-eclampsia (PE<37 weeks) when compared to normal control pregnancy. Therefore, soluble endoglin levels and soluble endoglin anti-angiogenic index levels (prior to 33 weeks) can not only be used for the diagnosis of premature pre-eclampsia, but also for the prediction of pre-eclampsia. It appears that elevations in soluble endoglin levels and soluble endoglin anti-angiogenic index levels start as early as 10-12 weeks prior to symptoms of pre-eclampsia.

The soluble endoglin levels and soluble endoglin anti-angiogenic index levels were also significantly elevated in term pre-eclampsia (PE>37 weeks) and modestly elevated in gestational hypertension and severe SGA when measured late in pregnancy (33-36 week gestational windows). Therefore, soluble endoglin levels and soluble endoglin anti-angiogenic index levels can also be used to identify other pregnancy complications such as SGA and gestation hypertension when measured after 33 weeks of pregnancy.

### Example 7

### Involvement of Soluble Endoglin in the Pathogenesis of Pre-Eclampsia

We have shown that endoglin, a cell surface receptor for the pro-angiogenic protein TGF-β and expressed on endothelium and syncytiotrophoblast, is upregulated in pre-eclamptic placentas. We have also shown that in pre-eclampsia, excess soluble endoglin is released from the placenta into the circulation through shedding of the extracellular domain. The experiments described below were designed to test the hypothesis that soluble endoglin may synergize with sFlt1, an anti-angiogenic factor which binds placental growth factor (PlGF) and VEGF, to cause endothelial dysfunction.

### Materials and Methods

Reagents

Recombinant Human endoglin, human sFlt1, mouse endoglin, mouse sFlt1, human TGF-β1, human TGF-β3, mouse VEGF were obtained from R&D systems (Minneapolis, Minn.). Mouse monoclonal antibody (catalog #sc 20072) and polyclonal antibody (sc 20632) against the N-terminal region of human endoglin was obtained from Santa Cruz Biotechnology, Inc. ELISA kits for human sFlt1, mouse sFlt1 and human soluble endoglin were obtained from R&D systems, MN.

Generation of Adenoviruses

Adenoviruses against sFlt1 and control adenovirus (CMV) have been previously described (Maynard et al, J. Clin. Invest. 111: 649:658 (2003)) and were generated at the Harvard Medical Core facility in collaboration with Dr. Richard Mulligan. To create the soluble endoglin adenovirus, we used the Adeasy Kit (Stratagene). Briefly, human soluble endoglin (encoding the entire extracellular region of the endoglin protein) was PCR amplified using human cDNA full length endoglin clone (Invitrogen, CA) as the template and the following oligonucleotides as primers: forward 5′-ACG AAG CTT GAA ACA GTC CAT TGT GAC CTT-3′ (SEQ ID NO: 3) and reverse 5′TTA GAT ATC TGG CCT TTG CTT GTG CAA CC-3′ (SEQ ID NO: 4). Amplified PCR fragments were initially subcloned into pSecTag2-B (Invitrogen, CA) and the DNA sequence was confirmed. A mammalian expression construct encoding His-tagged human soluble endoglin was PCR amplified using pSecTag2 B-soluble endoglin as the template and subcloned into pShuttle-CMV vector (Stratagene; Kpn1 and Sca1 sites), an adenovirus transfer vector, for adenovirus generation. Adenovirus expressing soluble endoglin (sE) was then generated using the standard protocol per manufacturer instructions and confirmed for expression by western blotting. The confirmed clone was then amplified on 293 cells and purified on a CsCl2 density gradient as previously described (Kuo et al, Proc. Natl. Acad. Sci. USA 98:4605-4610 (2001)). The final products were titered by an optical absorbance method (Sweeney et al, Virology, 2002, 295:284-288). The titer is expressed as plaque forming units (pfu)/mL based on a formula derived from previous virus preps that were titered using the standard plaque dilution based titration assay kit (BD Biosciences Clontech, Palo Alto, Calif., Cat. No. K1653-1) and the optical absorbance method.

Western Blots

Western blots were used for checking the expression of adenoviral-infected transgenes in the rat plasma as described elsewhere (Maynard et al, supra).

Immunoprecipitation (IP) Experiments

IP followed by western blots were used to identify and characterize soluble endoglin in the placental tissue and serum specimens from patients with pre-eclampsia. Human placental tissue was washed with cold PBS and lysed in homogenization buffer [10 mM Tris-HCl, pH 7.4; 15 mM NaCl; 60 mM KCl; 1 mM EDTA; 0.1 mM EGTA; 0.5% Nonidet P-40; 5% sucrose; protease mixture from Roche (Indianapolis, Ind.)] for 10 minutes. Placental lysates were then subjected to immunoprecipitation with an anti-human monoclonal mouse endoglin antibody (Santa Cruz Biotechnology, Inc., Santa Cruz, Calif.). Immunoaffinity columns were prepared by the directional coupling of 3-5 mg of the purified antibody to 2 ml protein A-Sepharose using an immunopure IgG orientation kit (Pierce Chemical Co., Rockford, Ill., USA) according to the manufacturer's instructions. Columns were then washed extensively with RIPA buffer containing protease mixture, and bound proteins were eluted with 0.1 mol/L glycine-HCl buffer, pH 2.8. The eluent was collected in 0.5-ml fractions containing 1 mol/L Tris-HCl buffer. Protein-containing fractions were pooled and concentrated 9- to 10-fold with CENTRICON Centrifugal Concentrator (Millipore Corp., Bedford, Mass., USA). The immunoprecipitated samples were separated on a 4-2% gradient gel (Invitrogen) and proteins were transferred to polyvinylidene difluoride (PVDF) membranes. Endoglin protein was detected by western blots using rabbit polyclonal antibody to human endoglin (Santa Cruz Biotechnology, Inc., Santa Cruz, Calif.).

Endothelial Tube Assay

Growth factor reduced matrigel (7 mg/mL, Collaborative Biomedical Products, Bedford, Mass.) was placed in wells (100 l/well) of a pre-chilled 48-well cell culture plate and incubated at 37° C. for 30 minutes to allow polymerization. HUVEC cells (30,000+ in 300 μl of endothelial basal medium with no serum, Clonetics, Walkersville, Md.) were treated with various combinations of recombinant protein (soluble endoglin, sFlt1, or both) and plated onto the Matrigel coated wells, and incubated at 37° C. for 12-16 hours. Tube formation was then assessed through an inverted phase contrast microscope at 4× (Nikon Corporation, Tokyo, Japan) and quantitatively analyzed (tube area and total length) using the Simple PCI imaging analysis software.

Microvascular Permeability Experiments

Balb-C mice were injected through the retro-orbital venous plexus with 1×10⁸ pfu of adenovirus expressing GFP or soluble endoglin or sFlt1 or combinations and microvascular permeability assay was performed 48 hours later. Mice were anesthetized by IP injection of 0.5 ml Avertin. 100 ml of 1% Evans blue dye (in PBS) was injected into the tail vein. 40 minutes later, mice were perfused via heart puncture with PBS containing 2 mM EDTA for 20 minutes. Organs (brain, lung, liver, kidney) were harvested and incubated in formamide for 3 days to elute Evans blue dye. OD of formamide solution was measured using 620 nm wave length.

Renal Microvascular Reactivity Experiments

Microvascular reactivity experiments were done as described previously (Maynard et al., supra) using rat renal microvessels (70-170 μm internal diameter). In all experimental groups, the relaxation responses of kidney microvessels were examined after pre-contraction of the microvessels with U46619 (thromboxane agonist) to 40-60% of their baseline diameter at a distending pressure of 40 mmHg. Once the steady-state tone was reached, the responses to various reagents such as TGF-β1 or TGF-03 or VEGF were examined in a standardized order. All drugs were applied extraluminally.

Animal Models

Both pregnant and non-pregnant Sprague-Dawley rats were injected with 2×10⁹ pfu of adenoviruses (Ad CMV or Ad sFlt1 or Ad sE or Ad sFlt1+Ad sE) by tail vein injections. Pregnant rats were injected at day 8-9 of pregnancy (early second trimester) and blood pressure measured at day 16-17 of pregnancy (early third trimester). Blood pressures were measured in the rats after anesthesia with pentobarbital sodium (60 mg/kg, i.p.). The carotid artery was isolated and cannulated with a 3-Fr high-fidelity microtip catheter connected to a pressure transducer (Millar Instruments, Houston, Tex.). Blood pressure was recorded and averaged over a 10-minute period. Blood, tissue and urine samples were then obtained before euthanasia. Plasma levels were measured on the day of blood pressure measurement (day 8 after injection of the adenoviruses), recognizing that 7-10 days after adenoviral injection corresponds to the peak level of expression of these proteins. Circulating sFlt-1 and soluble endoglin levels were confirmed initially by western blotting and then quantified using commercially available murine ELISA kits (R & D Systems, Minneapolis, Minn.). Urinary albumin was measured both by both standard dipstick and quantified by competitive enzyme-linked immunoassay using a commercially available rat albumin ELISA kit (Nephrat kit, Exocell Inc, Philadelphia, Pa.). Urinary creatinine was measured by a picric acid colorimetric procedure kit (Metra creatinine assay kit, Quidel Corp, San Diego, Calif.). AST and LDH were measured using the commercially available kits (Thermo Electron, Louisville, Colo.). Platelet counts from rat blood were measured using an automated hemocytometer (Hemavet 850, Drew Scientific Inc, Oxford, Conn.). A peripheral smear of the blood with Wright's stain was performed for the detection of schistocytes in circulating blood. After the measurement of blood pressure and collection of specimens, the rats were sacrificed and organs harvested for histology. The litter was counted and individual placentas and fetuses weighed. Harvested kidneys were placed in Bouin's solution, paraffin embedded, sectioned and stained with H&E, PAS or Masson's trichrome stain.

Statistical Comparisons

Results are presented as mean±standard error of mean (SEM) and comparisons between multiple groups were made by analysis of variance using ANOVA. Significant differences are reported when p<0.05.

### Results

### Soluble Endoglin is an Anti-Angiogenic Molecule and Induces Vascular Dysfunction

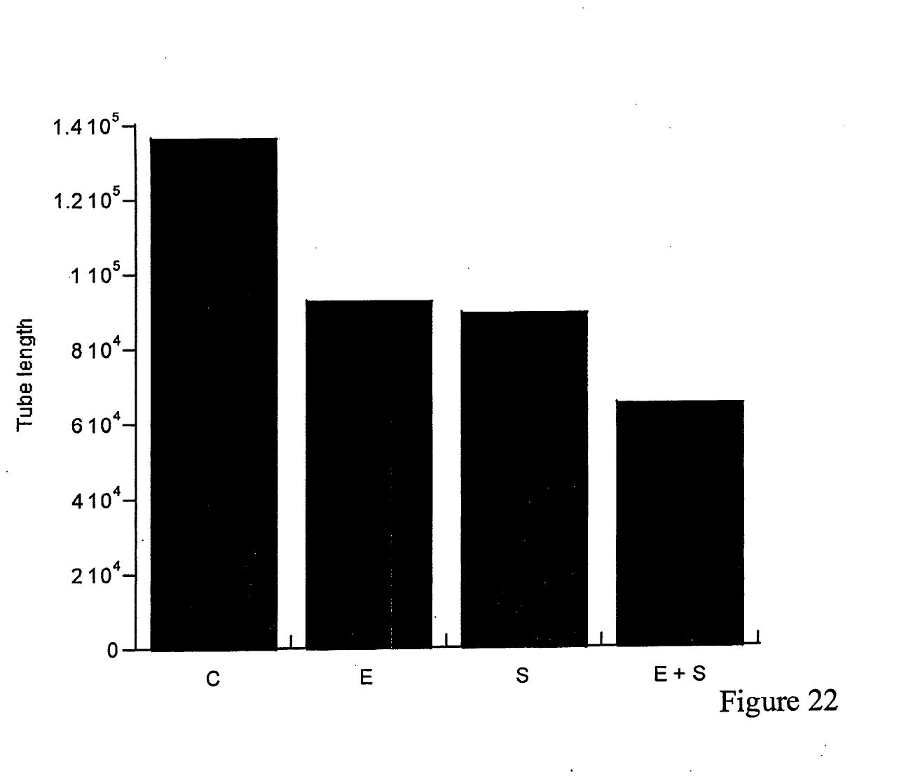

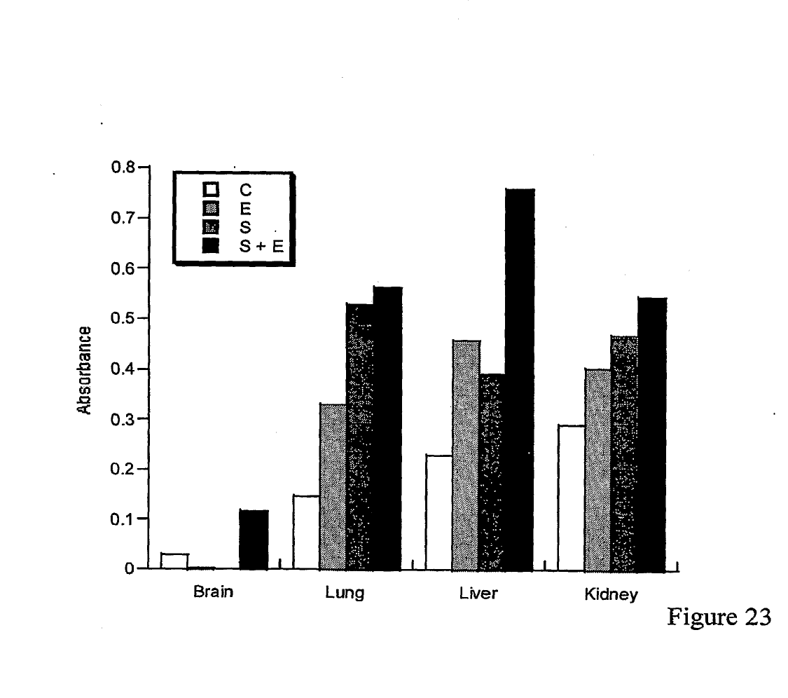

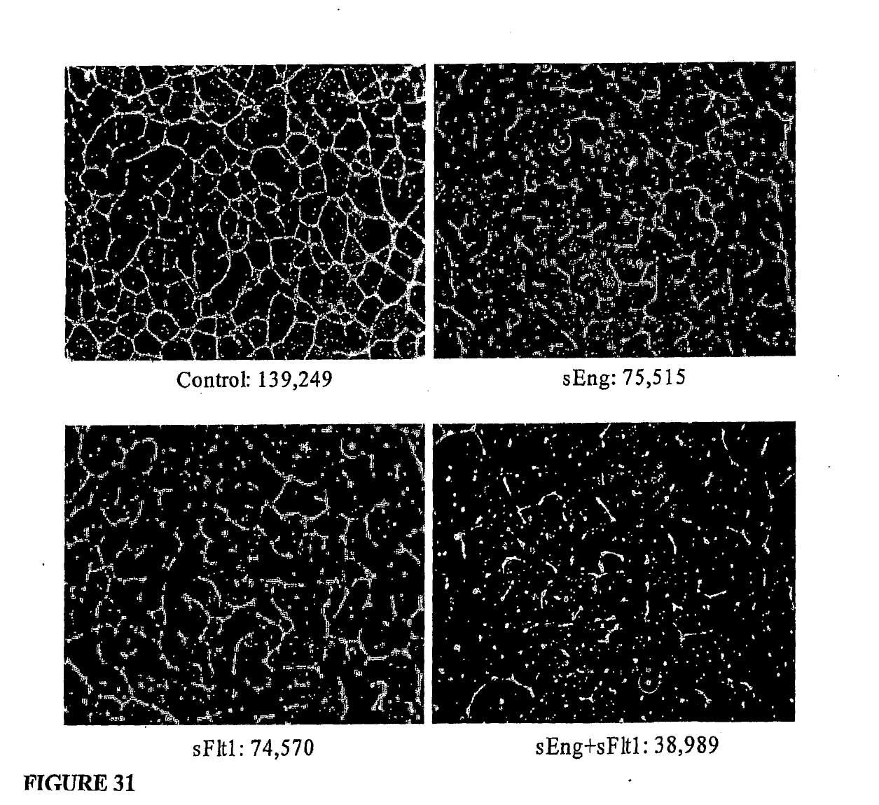

We used an in vitro model of angiogenesis to understand the function of the soluble endoglin. Soluble endoglin modestly inhibits endothelial tube formation, that is further enhanced by the presence of sFlt1 (FIG. 22 and FIG. 31). In pre-eclampsia, it has been reported that in addition to endothelial dysfunction, there is also enhanced microvascular permeability as evidenced by edema and enhanced leakage of Evan's blue bound albumin extracellularly. In order to see if soluble endoglin induces microvascular leak, we used mice treated for 48 hours with soluble endoglin and sFlt adenoviruses. A combination of soluble endoglin and sFlt1 induced a dramatic increase in albumin leakage in the lungs, liver and the kidney and a modest leakage in the brain as demonstrated using Evan's blue assay (FIG. 23). Soluble endoglin alone induced a modest leakage in the liver. Importantly, the combination of soluble endoglin and sFlt-1 showed an additive effect in the liver, indicating that these soluble receptors may act in concert to disrupt endothelial integrity and induce significant vascular damage and leak. These data suggest that soluble endoglin and sFlt1 combination are potent anti-angiogenic molecules and can induce significant vascular leakage.

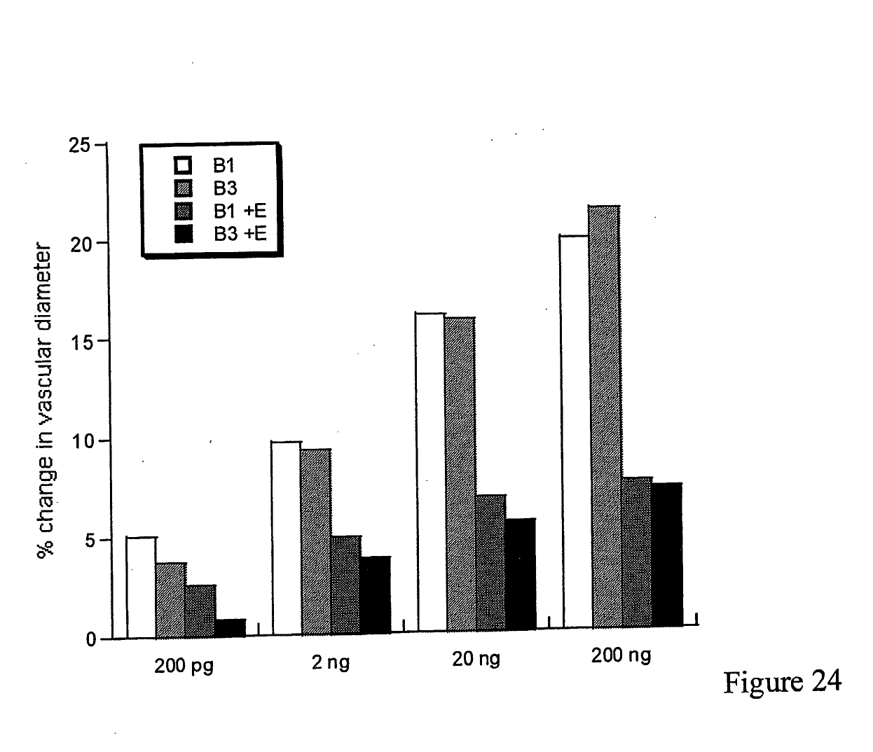

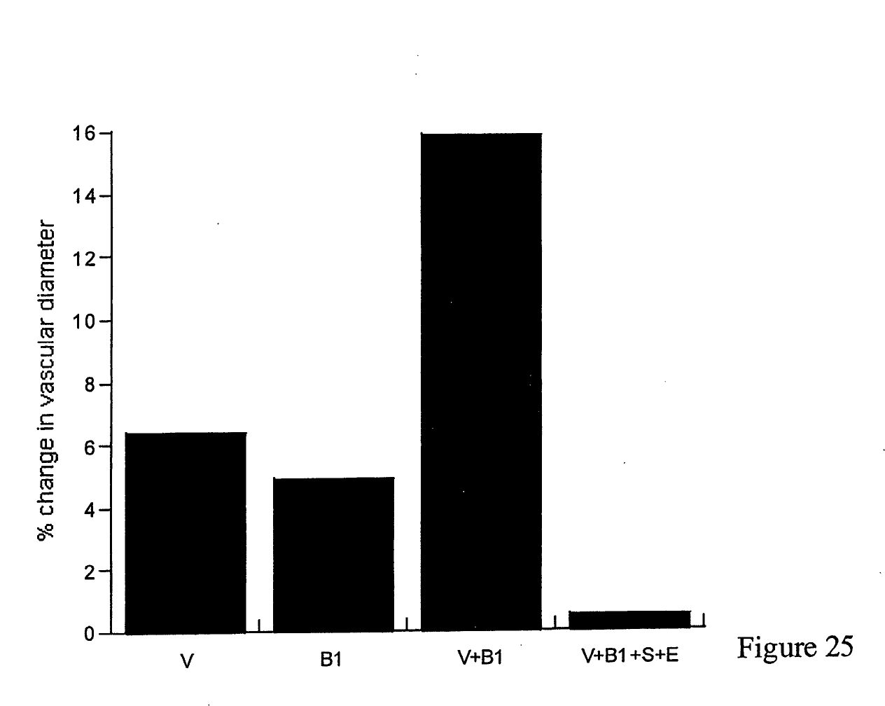

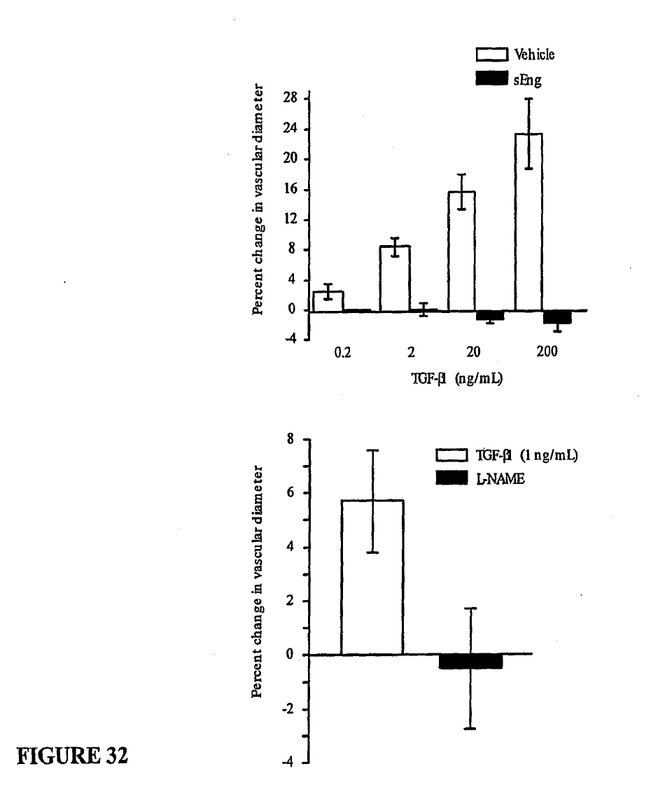

To assess the hemodynamic effects of soluble endoglin, a series of microvascular reactivity experiments in rat renal microvessels were performed. We studied first the effects of TGF-β1 and TGF-β3-two known ligands of endoglin. Both TGF-β1 and TGF-β3 induced a dose-dependent increase in vascular diameter. Both TGF-β1 and β3 induced a dose-dependent increase in arterial diameter, whereas, TGF-β2, which is not a ligand for endoglin, failed to produce any significant vasodilation (<2% at 0.1 and 1 μg/ml). Importantly in the presence of excess soluble endoglin, the effect of both the TGF-βs were significantly attenuated (FIG. 24). This acute effect of TGF-β1 and TGF-β3 isoforms on vascular tone was also seen in mesenteric vessels (FIG. 32). Finally, the combination of VEGF and TGF-β1 induced vasodilation which was blocked by excess soluble endoglin and sFlt1 (FIG. 25). This suggests that the sFlt1 and soluble endoglin may oppose the physiological vasodilation induced by angiogenic growth factors such as VEGF and TGF-β1 and induce hypertension.

In Vivo Effects of Soluble Endoglin and sFlt1

In order to assess the vascular effects of soluble endoglin and sFlt1, we resorted to adenoviral expression system in pregnant rats. Adenovirus encoding a control gene (CMV) or soluble endoglin or sFlt1 or sFlt1+soluble endoglin were injected by tail vein on day 8 of pregnancy in Sprague Dawley rats. On day 17, animals were examined for pre-eclampsia phenotype. Table 8 includes the hemodynamic and biochemical data.

TABLE 8
Hemodynamic and biochemical data for adenovirus treated rat animal
models.

| | | | Urine | Platelet | | | |
|---|---|---|---|---|---|---|---|
| | | MAP in | Alb/creat | count × | LDH | AST | Fetal |
| Groups | N | mmHg | μg/mg | 1000/μl | U/L | U/L | weight (g) |
| Control | 6 | 83 ± 5 | 186 ± 94 | 1,098 ± 75 | 156 ± 32 | 54 ± 4 | 2.1 ± 0.5 |
| (CMV) | | | | | | | |
| sFlt1 | 6 | 117 ± 7* | 2,295 ± 867* | 1,131 ± 91 | 172 ± 53 | 94 ± 4* | 1.75 ± 0.4 |
| SEng | 6 | 104 ± 6* | 432 ± 249 | 1,195 ± 78 | 188 ± 46 | 110 ± 13* | 1.6 ± 0.4 |
| sFlt1 + | 6 | 121 ± 9* | 9,029 ± 4043* | 615 ± 67* | 1,952 ± 784* | 210 ± 92* | 0.75 ± 0.3* |
| sEng | | | | | | | |

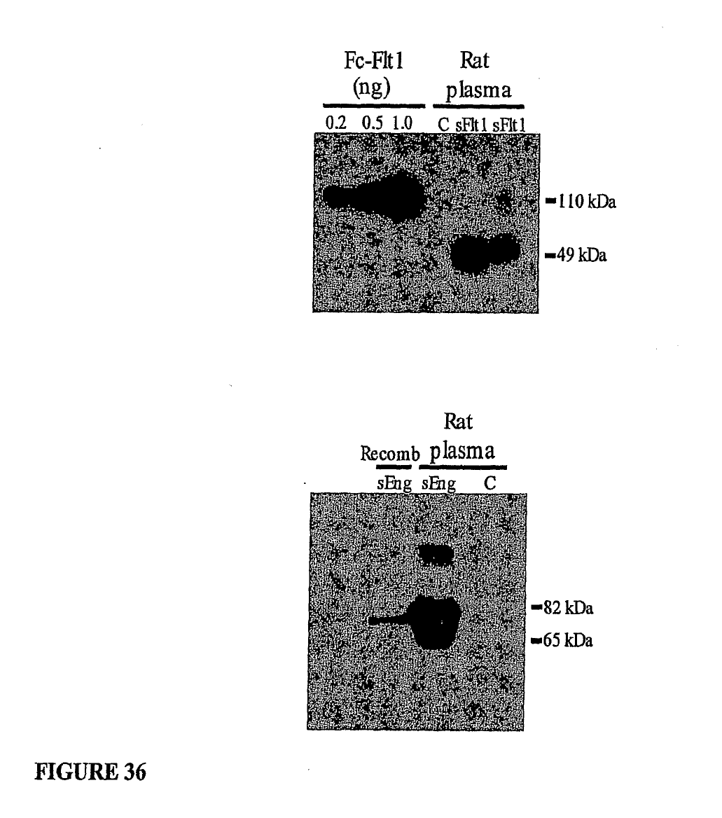

Data are presented as mean ± s.e.m.
MAP—mean arterial pressure (diastolic pressure + ⅓ pulse pressure);
Fetal weight is the average weight of the litter for each group in grams;
Alb/Creat—Albumin/creatinine ratios;
LDH—Lactate dehyrogenase;
AST—Aspartate Aminotransferase.
*P < 0.05 when compared to control group.
Expression of sFlt1 and sEng were first confirmed in rat plasma by Western blots (FIG. 36) and circulating concentration quantified using commercially available ELISA kits.
The mean plasma concentrations of sFlt1 in the control, sEng, sFlt1 and sFlt1 + sEng groups were 0.64 ng/ml, 0.66 ng/ml, 249 ng/ml, and 204 ng/ml respectively.
The concentrations of sEng in these four groups were 0.39 ng/ml, 129 ng/ml, 0.37 ng/ml, and 123 ng/ml, respectively.

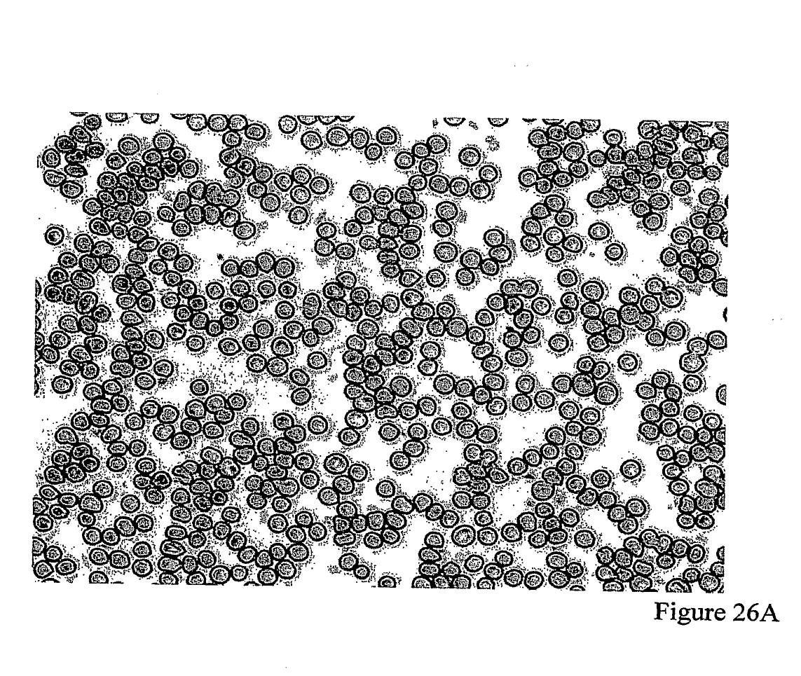

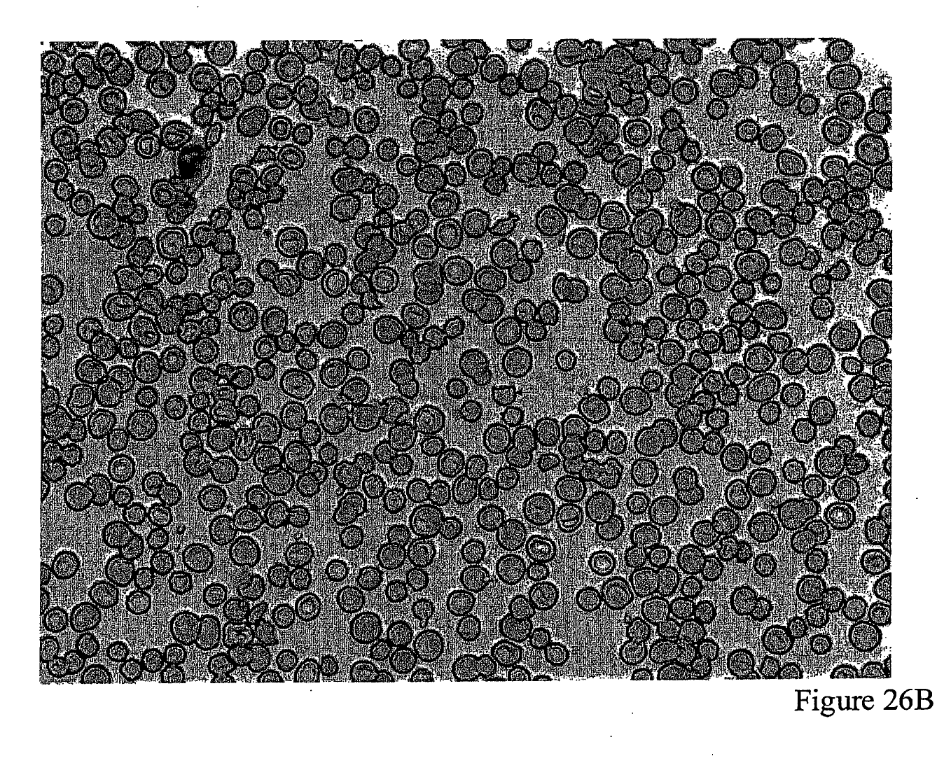

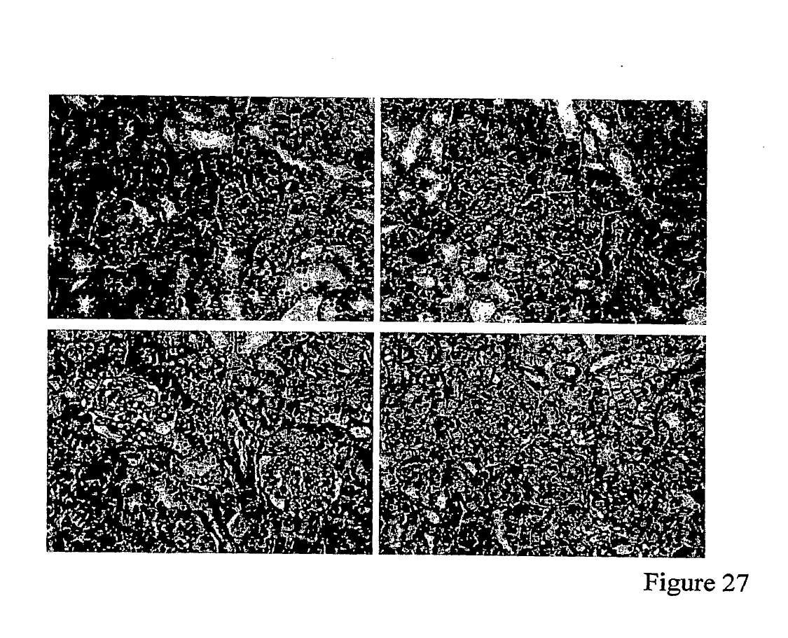

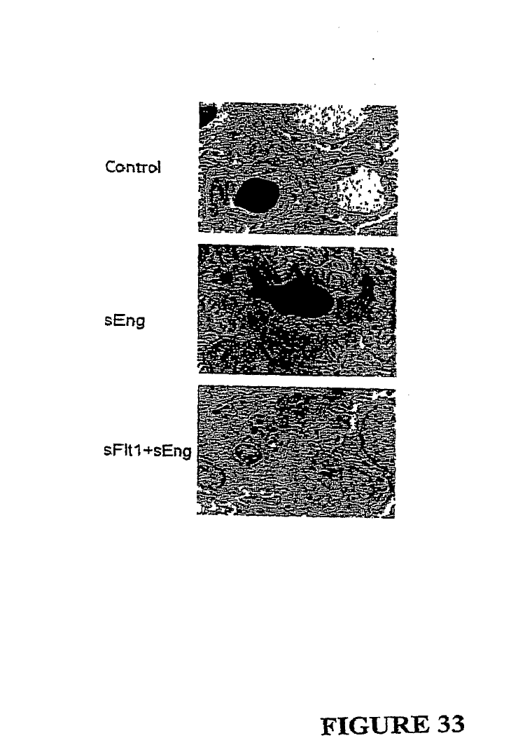

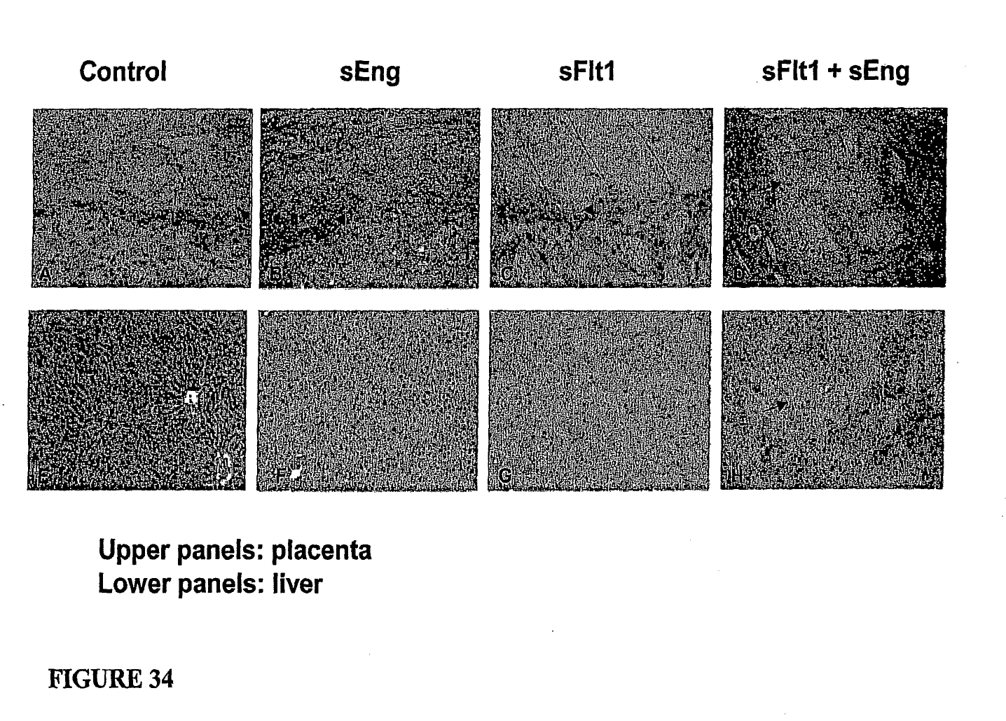

Soluble endoglin alone induced a mild hypertension. sFlt1 induced both hypertension and proteinuria, as previously reported. Fetal growth restriction was observed in litters born to the sFlt1+sEng group, probably related to the placental vascular ischemia and damage. Importantly, the combination of sFlt1 and soluble endoglin induced severe hypertension, nephrotic range proteinuria, growth restriction of the fetuses and biochemical evidence of the development of the HELLP syndrome (elevated LDH, elevated AST and decreasing platelet counts) (Table 8). Evidence of hemolysis in the soluble endoglin+sFlt1 group was confirmed by peripheral smear which revealed schistocytes and reticulocytosis (FIGS. 26A-B). Finally, renal histology also revealed focal endotheliosis in the soluble endoglin group and a severe glomerular endotheliosis in the soluble endoglin+sFlt1 group (FIGS. 27A-27D, and 33). Note that in FIG. 33, the control group is within normal limits. Note open capillary loops with fenestrated endothelium. The soluble endoglin panel shows endothelial swelling with loss of fenestrae and partial luminal occlusion. Note a red blood cell squeezing through the compromised lumen. While light microscopy of the kidneys of soluble endoglin treated rats was not striking for significant endotheliosis, electron microscopy revealed focal endotheliosis. Importantly, the animals that received both soluble endoglin and sFlt-1 had severe glomerular endotheliosis. The combination therapy group (lower panel) shows massive endocapillary occlusion with swollen endothelial cells. Note the relative preservation of podocyte foot processes (shown as arrows) despite severe proteinuria. Extensive vascular damage of the placenta including infarction at the maternal-fetal junction was observed in the sFlt1+sEng group, but not in control rats or in those treated with either agent alone (FIGS. 34A-H). Diffuse inflammation in the giant cell layer (corresponding to human invasive trophoblasts) was noted in the sFlt1 and sEng groups, and was higher in the combined group. Liver histology revealed signs of ischemia and areas of necrosis in the sFlt1+sEng group, similar to those seen in patients with the HELLP syndrome (FIGS. 34A-H). Signs of severe maternal vascular damage were also seen when sFlt1+sEng were injected to non-pregnant rats, suggesting that the observed phenotype in pregnant rats was due to a direct effect on the maternal vessels and did not require the placenta.

### Summary

These results demonstrate that soluble endoglin is up-regulated in pre-eclamptic placentas and is present at extremely high levels in patients with pre-eclampsia. The highest levels of soluble endoglin were present in patients with HELLP syndrome, one of the most severe forms of pre-eclampsia. These results also demonstrate that soluble endoglin levels correlated with the elevated sFlt1 in pregnant patients and was higher in those patients in whom there is a higher circulating sFlt1 levels. In addition, the results indicate that soluble endoglin is an anti-angiogenic molecule and disrupts endothelial function in multiple endothelial assays such as angiogenesis assays, microvascular permeability assays, and microvascular reactivity experiments. Importantly, soluble endoglin can amplify the toxic consequence of sFlt1 in these in vitro endothelial assays. Further, in in vivo assays, adenoviral expression of soluble endoglin induces mild hypertension without any significant proteinuria. However, in the presence of sFlt1, soluble endoglin induces significant vascular damage as evidenced by the presence of severe hypertension, proteinuria, glomerular endotheliosis, development of the HELLP syndrome and fetal growth restriction.

The mechanism of soluble endoglin release is likely proteolytic cleavage of the extracellular region of the endoglin molecule. Specific proteases that are up-regulated in the pre-eclamptic tissue may serve as candidate molecules. One example would be the membrane type matrix metalloproteinase-1 (MT1-MMP) that has been shown to cleave betaglycan, a molecule that shares similarity to endoglin (Velasco-Loyden G et al, J. Biol. Chem. 279:7721-33 (2004)). Therefore, inhibitors of such proteases can serve as valuable targets for the treatment of pre-eclampsia.

### Example 8

### Soluble Endoglin Inhibits TGF-β1 and TGF-β3 Mediated NOS-Dependent Vasodilation

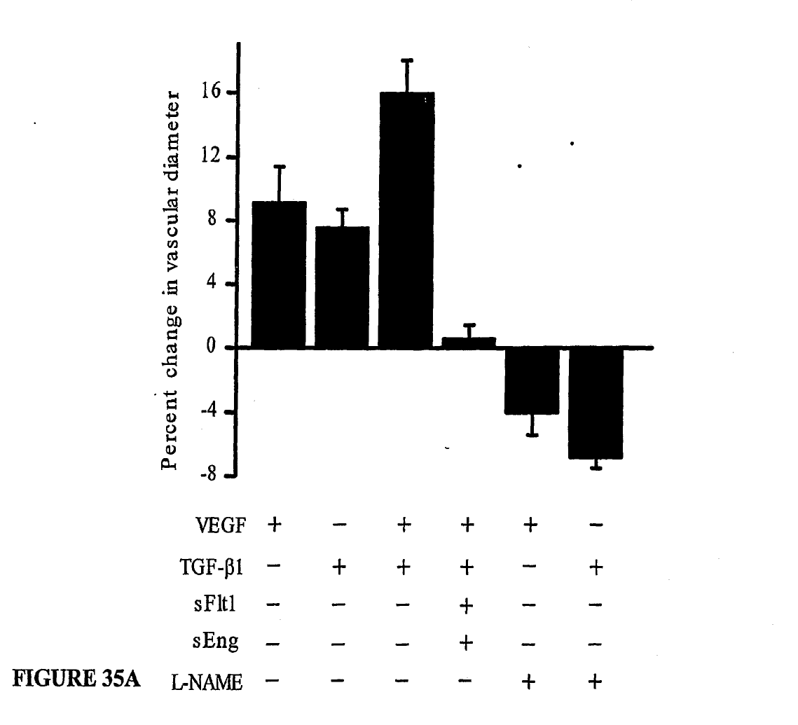

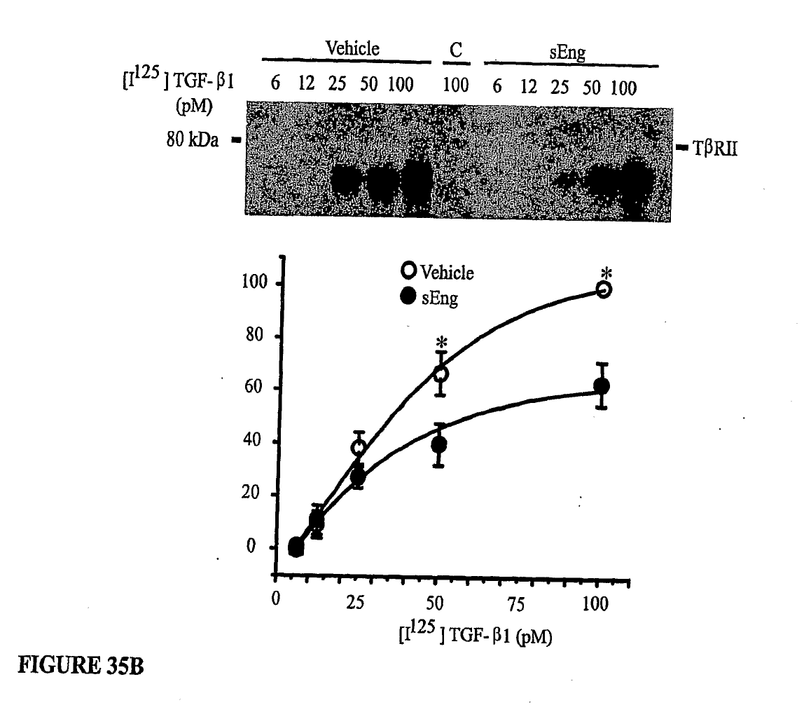

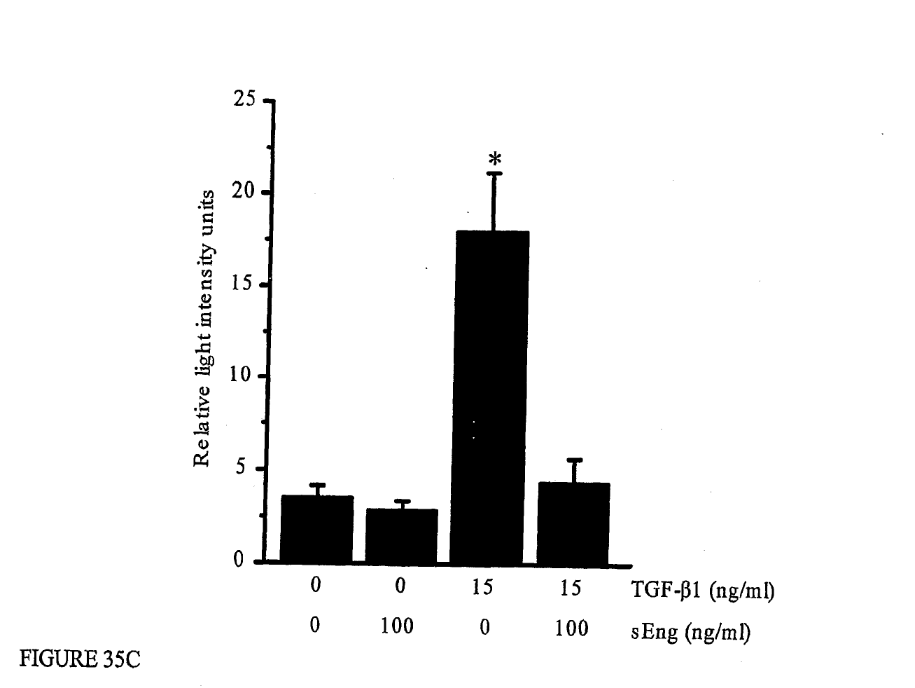

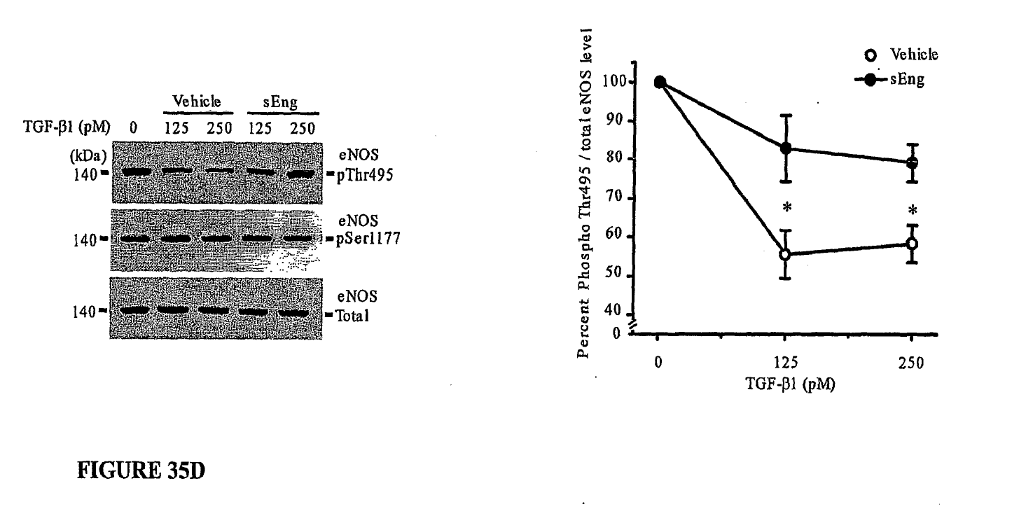

eNOS is a Ca²⁺/calmodulin-regulated nitric oxide (NO) synthase that can be activated by fluid shear stress and neurohumoral stimuli. Endothelium-derived NO is a very potent vasorelaxant contributing to systemic blood pressure regulation, vascular permeability, and angiogenesis. In fact, the effects of VEGF on angiogenesis and vascular tone are partly mediated by activation of eNOS, through increased eNOS/Hsp90 association and Akt-dependent eNOS phosphorylation at Ser1177. Our recent demonstration that increased placenta-derived sFlt1 in sera of preeclamptic patients is anti-angiogenic and induces hypertension may in fact reflect impaired VEGF-dependent eNOS activation (Maynard et al., supra). More recently, dephosphorylation of eNOS Thr495 has been shown to precede Ser1177 phosphorylation and these coordinated events determine eNOS activity in endothelial cells (Fleming et al., Cir. Res. 88:E68-75 (2001)). Given the known effect of VEGF on reducing vascular reactivity via eNOS activation and the recent demonstration that endoglin modulates eNOS-dependent vasomotor activity (Toporsian et al., Circ. Res. 96:684-692 (2005)), we assessed the hemodynamic effects of TGF-β isoforms and soluble endoglin in isolated rat renal microvessels. As described in Example 7 and in FIGS. 24 and 25, both TGF-β1 and -β3 induced a dose-dependent increase in arterial diameter which was significantly attenuated by soluble endoglin. This acute effect of TGF-β1 and -β3 isoforms on vascular tone has not been previously recognized and was also seen in mesenteric vessels (FIG. 32). VEGF and TGF-β1 had additive effects on vasodilation, which were blocked by sEng+sFlt1 at concentrations noted in patients with preeclampsia (FIGS. 25 and 35A). L-NAME blocked the vasodilation mediated by TGF-β1 and VEGF indicating a NOS dependent response (FIG. 35A). These data suggest that circulating sFlt1 and sEng may oppose the physiological NO-dependent vasodilatation elicited by these angiogenic growth factors, contributing to the development of hypertension seen in preeclampsia.

### Example 9

### Soluble Endoglin Inhibits TGF-β1 Binding and Signaling in Endothelial Cells

Given that endoglin is a co-receptor for TGF-β1 and -β3 isoforms, we hypothesized that soluble endoglin acts by interfering with cell surface receptor binding. Pre-incubating radio-labeled TGF-β1 with recombinant soluble endoglin significantly reduced its binding to TGF-β receptor type II (TβRII) at both 50 and 100 pM (FIG. 35B). Thus soluble endoglin competes for TGF-β1 binding to its receptors on endothelial cells. To test whether this leads to impaired signaling, the activity of a CAGA-Luc reporter construct was assessed in human endothelial cells. TGF-β1 induced the activation of the Smad 2/3-dependent CAGA-Luc reporter and this response was abolished by treatment with soluble endoglin (FIG. 35C).

### Example 10

### Soluble Endoglin Blocks TGF-β1 Mediated eNOS Activation

Given our findings that TGF-β1 induces a NOS-dependent vasorelaxation in both renal and mesenteric resistance vessels, we explored its immediate effects on eNOS activation. While TGF-β1 had no effect on eNOS Ser 1177 phosphorylation, it induced a significant dephosphorylation at Thr495 (FIG. 35D) suggesting that TGF-β regulates the phosphorylation status of a key residue in eNOS activation. This effect was significantly attenuated by soluble endoglin (FIG. 35D).

Taken together, the results in Examples 8-10 demonstrate that soluble endoglin interferes with TGF-β receptor binding and downstream signaling in endothelial cells and attenuates eNOS activation. Soluble endoglin and sFlt-1 may be working in concert to inhibit endothelial dependent NO activation and vasomotor effects by both the VEGF and the TGF-β signaling pathways.

### Example 11

### Sequential Changes in Angiogenic Factors can Identify Women at Risk for Pre-Eclampsia or Eclampsia

In the examples described above, we have shown that both sFlt1 and soluble endoglin (sEng) are intimately related to the pathogenesis of preeclampsia. In the example described below we measured the concentrations of sFlt 1 and sEng in paired serum specimens collected in first and second trimesters from women followed prospectively during pregnancy and whose pregnancy outcomes were characterized in detail in order to determine if sequential changes of these markers between first and second trimesters are associated with the development of pre-eclampsia.

### Materials and Methods

### Study Population

We performed a prospective, nested case-control study of women who enrolled in the Massachusetts General Hospital Obstetrical Maternal Study (MOMS) whose methods have been described previously (Thadhani et al., Obstet. Gynecol. 97:515-20 (2001) and Wolf et al., Obstet. Gynecol. 98:757-62 (2001)). In brief, the MOMS cohort was established in 1998 for the prospective study of early gestational risk factors for adverse outcomes that occur later in pregnancy. Women who received prenatal care at Massachusetts General Hospital and affiliated health centers were eligible for inclusion in the cohort. For the current study, consecutive women with singleton gestations between Jun. 1, 2001, and May 1, 2003, who enrolled in the MOMS cohort at or before 12 weeks of gestation and who delivered after 20 weeks were eligible for inclusion. Cases (n=39) were defined as those with blood collections in the first and second trimester who subsequently developed pre-eclampsia, and controls (n=147) were consecutive contemporaneous women enrolled in the same cohort who delivered at term (>37 weeks) and remained normotensive, normoglycemic, and without evidence of proteinuria throughout pregnancy. Cases and controls were matched by age (±2 years) and body mass index (±1 kg/m²) given potential for confounding by these exposures (Thadhani et al., Obstet. Gynecol. 94:543-50 (1999)). All subjects provided written informed consent, and this study was approved by the Institutional Review Board of the Massachusetts General Hospital.

### Primary Exposures

Blood samples were collected at the first prenatal visit (11-13 wks) and again in the second trimester (17-20 wks) in all women. Following collection, samples were stored at −80 C for future analysis. The primary exposures were serum sFlt1 and sEng that were measured using commercial ELISA kits (R&D systems, Minneapolis, Minn.) (Maynard et al., supra and Venkatesha et al., Nat. Med. 12:642-9 (2006)). The intraassay precision coefficients of variation for sFlt1 and sEng were 3.5 and 3.2% respectively. The interassay precision coefficients of variation for sFlt1 and endoglin were 8.1 and 9.5% respectively. All samples were run in duplicate, and if more than 10% variation existed between duplicates, the assay was repeated, and averages were reported. All assays were performed by someone who was blinded to case status. Samples were randomly ordered for analysis.

### Covariates and Confounders

The electronic medical record (EMR), which is the medical record used at the Massachusetts General Hospital, provides clinical and demographic data that prospectively details the events of pregnancy through the early postpartum period. Specific information obtained from the EMR collected at baseline (first prenatal visit) and at all subsequent prenatal visits included age, race, height, weight, smoking status, gestational age estimated from the last menstrual period and verified by ultrasound, blood pressure, and the results of urine analysis and fetal gestational age estimation. All pregnancy outcome information is also entered in the EMR including results of glucose tolerance tests and other routinely measured laboratory values, and delivery characteristics such as birth weight, route of delivery, and diagnosis of preeclampsia.

### Primary Outcomes

All pregnancy outcomes were verified by detailed examination of medical records, including prenatal flow sheets and laboratory measurements. At each prenatal visit blood pressure was obtained from the right arm using standard sphygmomanometers with the woman in the seated position after 3-5 minutes of rest. For each patient the proper cuff size was selected based on right midarm circumference. Measurements of blood pressure that coincided with the timing of the first (systolic) and fifth (diastolic) Korotkoff sounds were recorded. All subjects for the current study had no history of preexisting hypertension or diabetes mellitus, initiated and completed their prenatal care and pregnancy within the MOMS network, delivered a live infant, and had no evidence of hypertension 6 weeks after delivery.

Preeclampsia was defined as systolic blood pressure elevation of at least 140 mm Hg or diastolic blood pressure of at least 90 mm Hg after 20 wk gestation, in association with proteinuria, either 2+ or greater by dipstick or at least 300 mg/24 h in the absence of urinary tract infection (ACOG Committee on Practice Bulletins—Obstetrics. ACOG practice bulletin. diagnosis and management of preeclampsia and eclampsia. Number 33, January 2002. Obstet. Gynecol. 99:159-67). Preeclamptics were analyzed as term preeclampsia (≧37 weeks) and preterm preeclampsia (<37 weeks).

### Statistical Analysis

Demographic and clinical characteristics were compared using Chi Square tests or Student's t test, as appropriate. Log transformation was needed for the primary exposures given their skewed distributions (Levine et al., (2004), supra, Levine et al., N. Engl. J. Med. 355:992-1005 (2006)). Primary exposures were examined as continuous variables, and with cut points and tertile analyses based on the natural distributions of the controls and simplified for clinical interpretation. Multiple regression analysis was performed using logistic regression techniques. All P values were two-tailed, and a P value <0.05 was considered statistically significant.

### Secondary Analysis of Angiogenic Markers in the CPEP Study

We also performed a secondary analysis of angiogenic factor changes during early pregnancy from the recently published nested case controlled study within the CPEP cohort, described above. We analyzed samples from normotensive women, women who developed preeclampsia prior to 37 weeks, and women who developed preeclampsia after 37 weeks at three different time intervals—10-12 weeks, 13-16 weeks, and 17-20 weeks.

### Results

### Demographic and Clinical Characteristics

Baseline and delivery characteristics of the MOMS study population are displayed in Table 9. There were no significant differences in age and body mass index between the two groups. Women who subsequently developed preeclampsia had higher systolic and diastolic blood pressures at the first prenatal visit. At the time of presentation of preeclampsia, systolic and diastolic blood pressures were higher in preeclamptic group as expected.

TABLE 9
Demographics

| | Normal | Preeclampsia | |
|---|---|---|---|
| | (n = 147) | (n = 39) | P value |
| Variable | | | |
| Age (yrs) | 31.4 ± 5.4 | 32.8 ± 5.4 | 0.06 |
| BMI(kg/m²) | 28.5 ± 6.3 | 29.8 ± 9.1 | 0.40 |
| Smoker (% Never) | 47% | 60% | 0.21 |
| Parity | 0.7 ± 0.9 | 0.8 ± 1.0 | 0.47 |
| Baseline | | | |
| characteristics | | | |
| DBP(mmHg) | 70 ± 7 | 75 ± 8 | <0.01 |
| SBP(mmHg) | 113 ± 7 | 120 ± 13 | <0.01 |
| UTP(mg/dl) | NA | 614 ± 547 | |
| Gestational age at | 39.3 ± 1.9 | 37.2 ± 2.3 | <0.001 |
| delivery | | | |
| Birth weight | 3444 ± 532 | 3300 ± 809 | 0.35 |
| Characteristics at | | | |
| presentation | | | |
| Maximum DBP | 78 ± 5 | 93 ± 7 | <0.01 |
| (mmHg) | | | |
| Maximum SBP | 122 ± 7 | 147 ± 10 | <0.01 |
| (mmHg) | | | |
| Maximum SPOT | 0.6 ± 0.3 | 1.4 ± 0.9 | <0.01 |
| (g/g) | | | |

BMI = body mass index; DBP = diastolic blood pressure; SBP = systolic blood pressure UTP = urine total protein in mg/L; SPOT = urine protein/creatinine ratio
Values are mean ± S.D.

First and Second Trimester Levels of sFlt1 and sEng in Normal and Preeclamptic Pregnancy

The mean serum levels of sFlt1 were higher in women with preeclampsia compared to women with normal pregnancies, 3.49±0.35 ng/ml versus 3.03±0.13 ng/ml respectively, in first trimester (P=NS). In the second trimester the mean serum levels of sFlt1 were significantly higher in preeclamptic group, mean value of 4.12±0.5 ng/ml compared to normal group 3.10±0.15 ng/ml (P<0.01) (Table 10).

The mean serum levels of sEng thought not significantly different in the first trimester were significantly altered in the second trimester among women with preeclampsia, as compared to normal women, 6.9±0.32 ng/ml versus 6.57±0.17 ng/ml (P=NS) in first trimester and 6.37±0.38 ng/ml versus 5.23±0.12 ng/ml in second trimester, (P=0.004), respectively (Table 10).

TABLE 10
Serum levels of sFlt and sEng.

| | Normal Pregnancy | | | Preeclampsia | | | |
|---|---|---|---|---|---|---|---|
| Angiogenic Factor | N | mean | SE | N | Mean | SE | P value |
| sFlt1 (ng/ml) | 147 | 3.03 | 0.13 | 39 | 3.496 | 0.35 | 0.14 |
| (first trimester) | | | | | | | |
| sFlt1 (ng/ml) | 144 | 3.10 | 0.15 | 35 | 4.12 | 0.5 | 0.01* |
| (second trimester) | | | | | | | |
| sEng (ng/ml) | 147 | 6.57 | 0.17 | 39 | 6.9 | 0.32 | 0.37 |
| (first trimester) | | | | | | | |
| sEng (ng/ml) | 144 | 5.23 | 0.12 | 35 | 6.37 | 0.38 | 0.004* |
| (second trimester) | | | | | | | |

### Sequential Changes in Angiogenic Factors

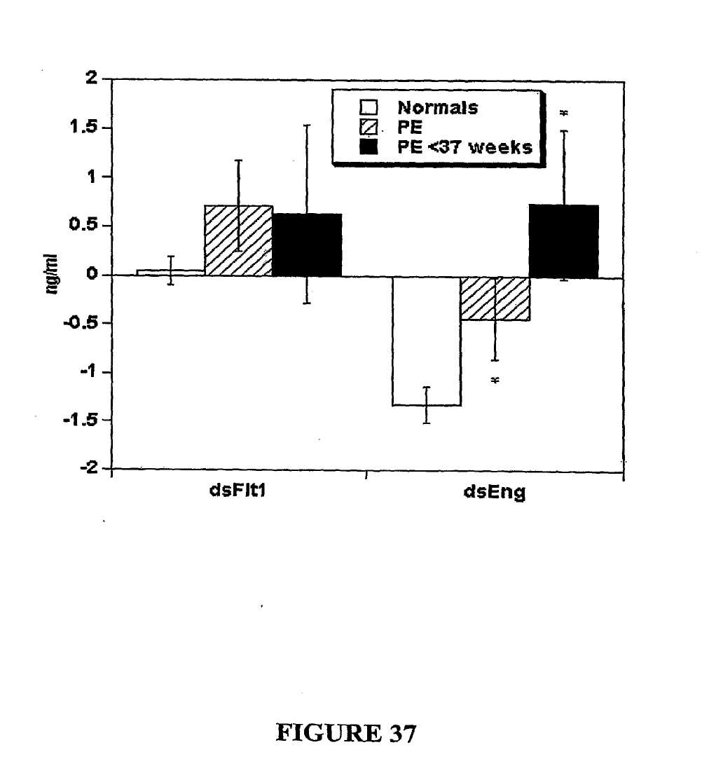

FIG. 37 graphically displays the delta or d (difference between first and second trimester values of sFlt1 and sEng) in normal, all preeclamptic women and in women with preeclampsia less than 37 weeks. In normal pregnancy, there is very little change in sFlt1 between first and second trimester (dsFlt1=0.05±0.15 ng/ml). dsFlt1 is relatively higher in women who develop pre-eclampsia 0.713±0.47 ng/ml versus 0.0497±0.15 ng/ml in normal women (P=0.08). Similarly in women with pre-eclampsia less than 37 weeks, dsFlt1 was higher at 0.634±0.91 ng/ml.

There is a fall in the level of sEng between the first and second trimester in normal pregnancy (−1.322±0.18 ng/ml). This fall is blunted in patients with pre-eclampsia, with dsEng of −0.441±0.42 ng/ml (P=0.04). In women with pre-eclampsia less than 37 weeks, the fall in the level of soluble endoglin is blunted and appeared to trend in the opposite direction (0.732±0.77 ng/ml, P<0.01 compared with controls).

### Predictive Algorithms

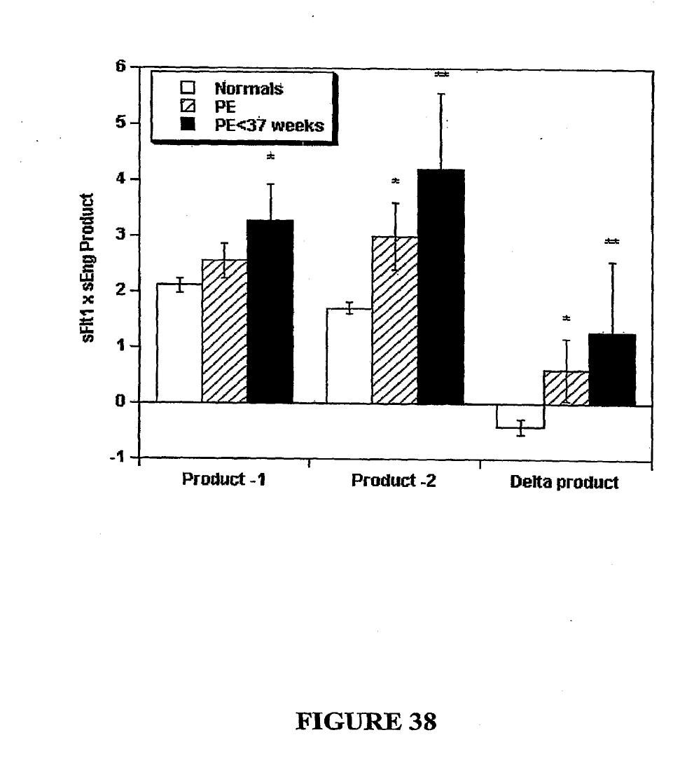

To see if these alterations can be used as a predictive test for severe premature pre-eclampsia, we looked at the product (value of sFlt1×sEng) as product-1 (in first trimester) and product-2 (in second trimester) in normotensive women and women who developed pre-eclampsia and specifically in preterm pre-eclampsia. Both product-1 and product-2 were significantly elevated in patients with pre-eclampsia and importantly the delta of the product (dproduct) were strikingly positive in contrast with a negative number in normal controls (FIG. 38). Furthermore, the dproduct were greatly amplified in patients with preterm pre-eclampsia as compared to normal controls (P=0.004).

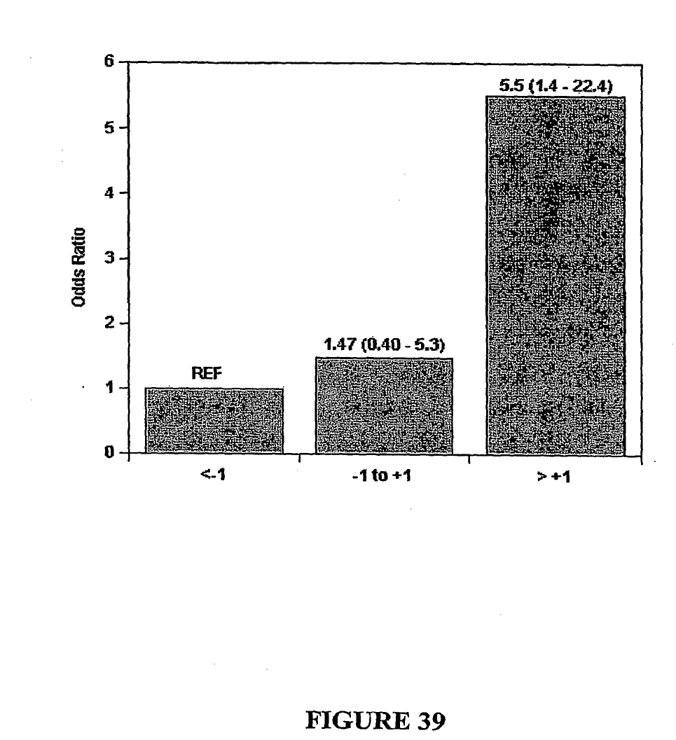

To assess the relationship between altered levels of angiogenic factors and risk of preterm pre-eclampsia, we computed adjusted odds ratios (aOR) and 95 percent confidence intervals (95% CI) for preterm pre-eclampsia in the highest category of the distribution of dproduct concentrations with respect to the lower two categories after adjustment for race/ethnicity, body-mass index, and gestational age at specimen collection (FIG. 39). Substantial increases in risk of preterm were observed in the group whose delta product levels were greater than +1 [aOR 5.5, 95% CI 1.4-22.4], compared to women whose delta product was less than −1.

### Secondary Analysis of the CPEP Nested Case Control Study

In the subset of women in the CPEP trial, among the cohort of women who remained normotensive, the mean values of sFlt1 increased from 3.68 ng/ml to 4.92 ng/ml between 10-12 wks and 13-16 weeks and decreased to 4.29 ng/ml at 17-20 weeks, while in women with preterm pre-eclampsia, the mean values of sFlt1 increased from 3.44 ng/ml to 4.22 ng/ml and further increased to 5.39 ng/ml at 10-12, 13-16 and 17-20 weeks of gestation. This pattern was not obvious in women with term pre-eclampsia (Table 11).

TABLE 11
Serum levels of sFlt and sEng in CPEP trial.

| | sFlt1 (ng/ml) | | | sEng (ng/ml) | | |
|---|---|---|---|---|---|---|
| | 10-12 | 13-16 | 17-20 | 10-12 | 13-16 | 17-20 |
| | wks | wks | wks | wks | wks | wks |
| Normal | 3.68 | 4.99 | 4.29 | 6.8 | 7.07 | 5.78 |
| N | 6 | 62 | 48 | 6 | 62 | 48 |
| PE < 37 wks | 3.44 | 4.22 | 5.39 | 7.15 | 7.9 | 10.19 |
| N | 13 | 28 | 32 | 13 | 28 | 32 |
| PE ≧ 37 wks | 4.06 | 4.48 | 4.25 | 7.41 | 7.8 | 8.34 |
| N | 17 | 50 | 48 | 17 | 50 | 48 |

Similar results were observed in the levels of sEng. In normotensive women the levels of sEng increased from 6.8 ng/ml to 7.07 ng/ml at 10-12 wks and 13-16 wks and then decreased to 5.78 ng/ml at 17-20 wks while in women with preterm preeclampsia the levels of endoglin increased from 7.15 ng/ml to 7.95 ng/ml and further increased to 10.19 ng/ml between 10-12 wks, 13-16 wks and 17-20 weeks. The same trend was noted in women with term preeclampsia where the mean levels of sEng increased from 7.41 ng/ml to 7.80 ng/ml to 8.34 ng/ml at 10-12 weeks, 13-16 weeks and 17-20 weeks (see Table 11).

### Summary

Both sFlt1 and sEng are elevated during second trimester in patients destined to develop pre-eclampsia. Normal pregnancy is characterized by a fall in sEng from first to second trimester without significant change in sFlt1. However, in patients who develop pre-eclampsia, particularly preterm pre-eclampsia, both sFlt1 and sEng continue to rise from first to second trimester. The changes in sFlt1 and sEng during first and second trimesters are useful for screening patients at high risk for subsequent development of preterm pre-eclampsia.

These findings have important implications for the prediction of preterm preeclampsia. The pursuit of a safe, reliable screening test for preeclampsia has been a goal of researchers for many years. Previous efforts have focused on detecting early manifestations of disease such as microalbuminuria, weight gain and plasma volume changes. In a large metaanalysis, Conde-Agudelo A et al analyzed eighty-seven of 7,191 (211,369 women) potentially relevant articles to assess the usefulness of clinical, biophysical, and biochemical tests in the prediction of pre-eclampsia. They concluded that as of 2004, there was no clinically useful screening test for predicting the development of pre-eclampsia (12). In the present study we have shown that the levels of sFlt1 and sEng are elevated in women who are destined to become pre-eclamptic, in their first and second trimester (as measured as a delta in individual patients), weeks to months before the clinical onset of disease. These changes are more substantial in women who develop preterm pre-eclampsia.

An imbalance in angiogenic factors is thought to play an intimate role in the pathogenesis of pre-eclampsia. Pre-eclamptic placentas are characterized by shallow implantation and abnormal vascular remodeling including impaired pseudo-vasculogenesis (Fisher et al., Semin Cell Biol. 4:183-8 (1993)). It is believed that these placental changes occur between 12-18 weeks of pregnancy and is important in the pathogenesis of the vast majority of severe early onset preeclampsia. It is thought that these placentation abnormalities lead to the elaboration of systemic factors that induce the maternal syndrome of preeclampsia. As described herein and in PCT application publication numbers WO 2004/008946, WO 2005/077007, and WO 2006/034507, both sFlt1 and sEng, two anti-angiogenic proteins have been found to be elevated in preeclampsia not only during clinical disease but also several weeks before onset of symptoms (Levine et al., (2006), supra). Importantly, both factors have been implicated in inducing a preeclampsia-like syndrome in rats (Maynard et al., supra, Venkatesha et al., supra). However, since alterations in concentrations of angiogenic factors in the maternal circulation occur relatively late in pregnancy, increased production of these anti-angiogenic factors may be a secondary phenomenon that occurs in response to abnormal placentation. In vitro data using placental villous explants and primary cytotrophoblast culture studies suggest that in addition to its role in inducing maternal endothelial dysfunction, anti-angiogenic factors may be involved in cytotrophoblast migration and differentiation. Our findings that levels of anti-angiogenic factors decrease from the first to the second trimester in normal pregnancies, but not in pregnancies in which preterm preeclampsia later develops, suggests that abnormalities of circulating angiogenic factors are occurring at the same time as abnormalities in placental differentiation.

The etiology of the increased concentrations of circulating sFlt1 and sEng in preeclamptic patients is unknown. Hypoxia, genes, or immunological factors are believed to play a role. It is worth noting that expression of both sFlt1 and sEng are elevated in response to hypoxia in vivo and in vitro models of placental hypoxia where increased expression is mediated by HIF-1 (Nevo et al., Am J Physiol Regul Integr Comp Physiol. 291:R1085-93 (2006)). Furthermore, it is believed that during normal pregnancy the placenta is hypoxic early in pregnancy and this hypoxia disappears with increased blood flow to the placenta during second trimesters. Although hypoxia has never been formally documented in pre-eclamptic pregnancies, it is believed that hypoxia is central to most pre-eclamptic pregnancies based on surrogate evidence of increased hypoxia induced transcription factor expression and impaired Doppler blood flow to the placentas. Our findings that both sFlt1 and sEng remain elevated in patients with severe pre-eclampsia in contrast to normal pregnancies where there is a fall between first and second trimesters suggests that placental ischemia may in fact play a role in the increased production of these anti-angiogenic proteins in pre-eclamptic patients.

In summary, sequential changes of sFlt1 and sEng appear to identify women destined to develop preeclampsia, especially women who subsequently develop preterm pre-eclampsia. Our findings are reproduced in cross-sectional studies with much larger sample size (Table 11).

### Example 12

### Endoglin is Necessary for TGF-β1-Induced eNOS Thr495 Dephosphorylation

Murine endothelial cells were derived from Eng^{+/+} and Eng^{-/-} mouse embryos (E8.5) and grown as described in Balconi et al. (Arterioscler. Thromb. Vasc. Biol. 20:1443-51 (2000)). Confluent monolayers were serum starved for 2 hours and stimulated with or without TGF-β1 (125 and 250 pM) for 15 minutes. Cell extracts were immediately prepared in 10 mM Tris-HCl containing 1% Triton X-100 and supplemented with protease and phosphatase inhibitors. Protein concentrations were quantified and samples were analyzed by western blot using phospho-specific pAbs to Thr495 of eNOS (Cell Signaling) and a mAb for total eNOS (BD Biosciences).

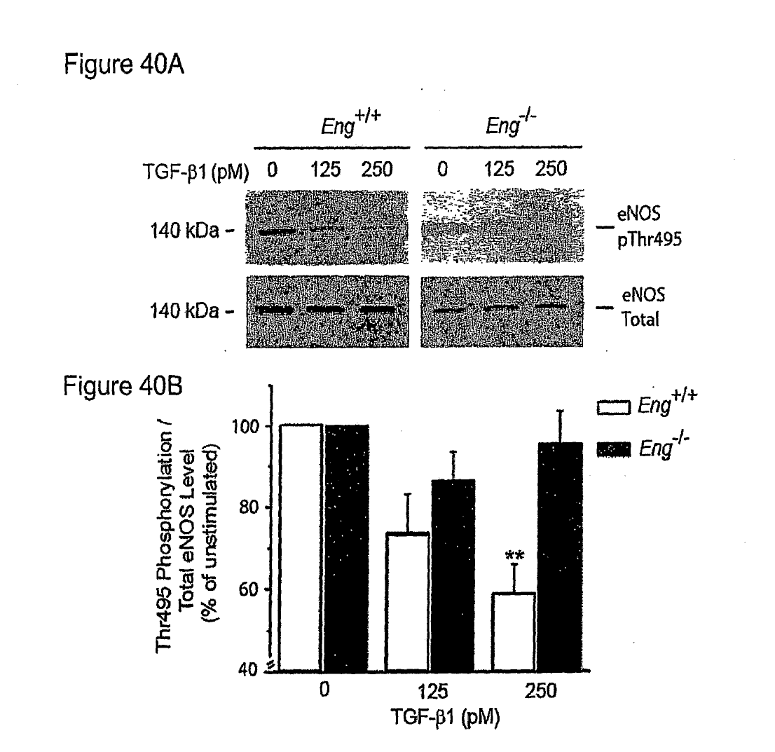

The representative western blots shown in FIG. 40A and associated graph shown in FIG. 40B (mean of n=3 experiments) demonstrate that TGF-β1-induced (**P<0.01 versus baseline) eNOS Thr495 dephosphorylation in mouse Eng^{+/+} endothelial cells but not in Eng^{-/-} cells. This result suggests a critical role for endoglin in linking TGF-β1 signaling to eNOS Thr495 dephosphorylation and activation. Moreover, given that this process is endoglin-dependent, soluble endoglin can be used to inhibit the process, presumably by binding to and inhibiting TGF-β1.

### Other Embodiments

The description of the specific embodiments of the invention is presented for the purposes of illustration. It is not intended to be exhaustive or to limit the scope of the invention to the specific forms described herein. Although the invention has been described with reference to several embodiments, it will be understood by one of ordinary skill in the art that various modifications can be made without departing from the spirit and the scope of the invention, as set forth in the claims.

All patents, patent applications, and publications referenced herein, including PCT Application Publication Numbers WO 2004/008946, WO 2005/077007, and WO 2006/034507; U.S. Patent Application Publication Numbers 20060067937 and 20070104707; and U.S. Provisional Patent Application No. 60/852,761 are hereby incorporated by reference.

Other embodiments are in the claims.

What is claimed is:

1-57. (canceled)

58. A method of diagnosing a subject as having, or having a predisposition to, a pregnancy related hypertensive disorder, said method comprising measuring the level of a soluble endoglin polypeptide and an sFlt-1 polypeptide from said subject and calculating the relationship between said levels of soluble endoglin and sFlt-1 using a [soluble endoglin×sFlt-1] metric, wherein an increase in the metric value in the subject sample relative to the metric value in a normal reference sample, is a diagnostic indicator of, or a propensity to develop, a pregnancy related hypertensive disorder in said subject.

59. The method of claim 58, wherein said metric further comprises the body mass index of the mother or the gestational age of the fetus.

60. The method of claim 58, wherein said sample is a bodily fluid, cell, or a tissue of said subject in which said soluble endoglin is normally detectable.

61. The method of claim 60, wherein said bodily fluid is selected from the group consisting of urine, amniotic fluid, blood, serum, and plasma.

62. The method of claim 60, wherein said cell is selected from the group consisting of an endothelial cell, a leukocyte, and a cell derived from the placenta.

63. The method of claim 62, wherein said leukocyte is a monocyte.

64. The method of claim 60, wherein said tissue is a placental tissue.

65. The method of claim 58, wherein said subject is a non-pregnant human, a pregnant human, a post-partum human, or a non-human and said method diagnoses a propensity to develop a pregnancy related hypertensive disorder.

66. The method of claim 65, wherein said non-human is selected from the group consisting of a cow, a horse, a sheep, a pig, a goat, a dog, or a cat.

67. The method of claim 58, wherein said pregnancy related hypertensive disorder is pre-eclampsia, eclampsia, chronic hypertension, HELLP syndrome, gestational hypertension, or pregnancy with an SGA infant.

68. The method of claim 67, wherein said pre-eclampsia is pre-term pre-eclampsia.

69. The method of claim 58, further comprising measuring the level of free placental growth factor polypeptide (PlGF) in said sample from said subject, wherein a decrease in said level of free PlGF is a diagnostic indicator of a pregnancy related hypertensive disorder in said subject.

70. The method of claim 58, further comprising measuring the level of free PlGF in said sample from said subject and calculating the relationship between said levels of soluble endoglin, sFlt-1, and free PlGF using a [(soluble endoglin+sFlt-1)/PlGF] metric, wherein an increase in the metric value in the subject sample relative to the metric value in a normal reference sample, is a diagnostic indicator of a pregnancy related hypertensive disorder in said subject.

71. The method of claim 58, wherein said normal reference is a prior sample or level from said subject.

72. The method of claim 71, wherein said reference sample is taken during the first trimester of pregnancy.

73. The method of claim 58, wherein said normal reference sample is a sample taken from a subject that is pregnant but does not have pre-eclampsia or eclampsia, or a propensity to develop pre-eclampsia or eclampsia.

74. The method of claim 58, wherein said subject is in the second trimester of pregnancy.

75. The method of claim 58, wherein said subject is in the third trimester of pregnancy.

76. The method of claim 58, wherein the method diagnoses said pregnant human subject as having a propensity to develop pre-eclampsia or eclampsia or pre-term pre-eclampsia or eclampsia.

77. A method of diagnosing a subject as having, or having a predisposition to, a pregnancy related hypertensive disorder, said method comprising measuring the level of a soluble endoglin polypeptide and an sFlt-1 polypeptide from said subject and calculating the relationship between said levels of soluble endoglin and sFlt-1 using the following metric: [dproduct=(sFlt×soluble endoglin) in the second trimester−(sFlt×soluble endoglin) in the first trimester] wherein a dproduct value greater than zero is a diagnostic indicator of a pregnancy related hypertensive disorder in said subject.

78. The method of claim 77, wherein a dproduct value greater than one is a diagnostic indicator of a pregnancy related hypertensive disorder in said subject.

79. The method of claim 77, wherein said pregnancy related hypertensive disorder is pre-eclampsia, eclampsia, chronic hypertension, HELLP syndrome, gestational hypertension, or pregnancy with an SGA infant.

80. The method of claim 79, wherein said pre-eclampsia is pre-term pre-eclampsia.

81. The method of claim 80, wherein a dproduct value greater than one is a diagnostic indicator of pre-term pre-eclampsia.

82. An antibody or antigen-binding fragment thereof that specifically binds a soluble endoglin polypeptide, wherein said antibody binds to an epitope comprising amino acids 40 to 86, 144 to 199, 206 to 222, 289 to 304, or 375 to 381 of the human endoglin sequence shown in FIG. 30B.

83. The antibody or antigen-binding fragment thereof of claim 82, wherein said antibody or antigen-binding fragment prevents binding of a growth factor to soluble endoglin.

84. The antibody or antigen-binding fragment thereof of claim 83, wherein said growth factor is selected from the group consisting of TGF-β1, TGF-β3, activin A, BMP-2, and BMP-7.

85. The antibody or antigen-binding fragment thereof of claim 82, wherein said antibody is a monoclonal antibody, chimeric antibody, humanized antibody, or human antibody.

86. The antibody or antigen-binding fragment thereof of claim 82, wherein said antibody or antigen-binding fragment thereof is present in a pharmaceutically acceptable carrier.

87. A kit for the diagnosis of a pregnancy related hypertensive disorder in a subject comprising the antibody of claim 82.
